SEQUENCE LISTING (81 sequences)

SEQ ID NO: 1 - DNA, 19 bp, synthetic construct
  source 1..19: mol_type = other DNA
ggacacggcctatagcctg

SEQ ID NO: 2 - DNA, 20 bp, synthetic construct
  source 1..20: mol_type = other DNA
ctcttggcgcagttcttgtc

SEQ ID NO: 3 - DNA, 21 bp, synthetic construct
  source 1..21: mol_type = other DNA
agggcagaatcatcacgaagt

SEQ ID NO: 4 - DNA, 20 bp, synthetic construct
  source 1..20: mol_type = other DNA
agggtctcgattggatggca

SEQ ID NO: 5 - DNA, 24 bp, synthetic construct
  source 1..24: mol_type = other DNA
tctcgtttccaatgcacgtacagc

SEQ ID NO: 6 - DNA, 23 bp, synthetic construct
  source 1..23: mol_type = other DNA
agtgacagcagcagttgcacagt

SEQ ID NO: 7 - DNA, 21 bp, synthetic construct
  source 1..21: mol_type = other DNA
ggaggccgagaatatcacgac

SEQ ID NO: 8 - DNA, 19 bp, synthetic construct
  source 1..19: mol_type = other DNA
ccctgccagacttctacgg

SEQ ID NO: 9 - DNA, 22 bp, synthetic construct
  source 1..22: mol_type = other DNA
ctcctgcaagagtttgatgtcc

SEQ ID NO: 10 - DNA, 20 bp, synthetic construct
  source 1..20: mol_type = other DNA
catgccgatgtcatggtagg

SEQ ID NO: 11 - DNA, 23 bp, synthetic construct
  source 1..23: mol_type = other DNA
attctcaacactccaaactgtgc

SEQ ID NO: 12 - DNA, 21 bp, synthetic construct
  source 1..21: mol_type = other DNA
actttagcttcgggtcaatgc

SEQ ID NO: 13 - DNA, 19 bp, synthetic construct
  source 1..19: mol_type = other DNA
gaacggctcgtcagaggtg

SEQ ID NO: 14 - DNA, 21 bp, synthetic construct
  source 1..21: mol_type = other DNA
acagtgcagattctcatcgcc

SEQ ID NO: 15 - DNA, 21 bp, synthetic construct
  source 1..21: mol_type = other DNA
gcctgggagcttacgattttg

SEQ ID NO: 16 - DNA, 20 bp, synthetic construct
  source 1..20: mol_type = other DNA
tagtgccctggtactggtcg

SEQ ID NO: 17 - DNA, 23 bp, synthetic construct
  source 1..23: mol_type = other DNA
agaaggtgaaacgatcatcgagc

SEQ ID NO: 18 - DNA, 21 bp, synthetic construct
  source 1..21: mol_type = other DNA
ctcatccataccaccggatct

SEQ ID NO: 19 - DNA, 22 bp, synthetic construct
  source 1..22: mol_type = other DNA
ccaggatggttcttagactccc

SEQ ID NO: 20 - DNA, 21 bp, synthetic construct
  source 1..21: mol_type = other DNA
tttagcacgaagctctccgat

SEQ ID NO: 21 - DNA, 18 bp, synthetic construct
  source 1..18: mol_type = other DNA
ctcaaggtcgtgcgtctg

SEQ ID NO: 22 - DNA, 22 bp, synthetic construct
  source 1..22: mol_type = other DNA
tggctttctctttcctcttctc

SEQ ID NO: 23 - DNA, 21 bp, synthetic construct
  source 1..21: mol_type = other DNA
catcctgggacgagatgaact

SEQ ID NO: 24 - DNA, 20 bp, synthetic construct
  source 1..20: mol_type = other DNA
tgacaagcgttaccacaggc

SEQ ID NO: 25 - DNA, 22 bp, synthetic construct
  source 1..22: mol_type = other DNA
actctccttgctactgattcct

SEQ ID NO: 26 - DNA, 21 bp, synthetic construct
  source 1..21: mol_type = other DNA
atcgtgacattttctgcctcc

SEQ ID NO: 27 - DNA, 21 bp, synthetic construct
  source 1..21: mol_type = other DNA
tctgctgggaacaactcaaca

SEQ ID NO: 28 - DNA, 22 bp, synthetic construct
  source 1..22: mol_type = other DNA
gtgagacacctcatcagggtat

SEQ ID NO: 29 - DNA, 21 bp, synthetic construct
  source 1..21: mol_type = other DNA
gggcaggttctggtattggat

SEQ ID NO: 30 - DNA, 19 bp, synthetic construct
  source 1..19: mol_type = other DNA
ggctcggaaatggtagggg

SEQ ID NO: 31 - DNA, 20 bp, synthetic construct
  source 1..20: mol_type = other DNA
gtaacccgttgaaccccatt

SEQ ID NO: 32 - DNA, 20 bp, synthetic construct
  source 1..20: mol_type = other DNA
ccatccaatcggtagtagcg

SEQ ID NO: 33 - DNA, 23 bp, synthetic construct
  source 1..23: mol_type = other DNA
tagtccttcctaccccaatttcc

SEQ ID NO: 34 - DNA, 21 bp, synthetic construct
  source 1..21: mol_type = other DNA
ttggtccttagccactccttc

SEQ ID NO: 35 - DNA, 21 bp, synthetic construct
  source 1..21: mol_type = other DNA
tgttctcctaacccgatccct

SEQ ID NO: 36 - DNA, 21 bp, synthetic construct
  source 1..21: mol_type = other DNA
caggaagctgcaaagatccca

SEQ ID NO: 37 - DNA, 23 bp, synthetic construct
  source 1..23: mol_type = other DNA
atgagtttttcccttatggggac

SEQ ID NO: 38 - DNA, 21 bp, synthetic construct
  source 1..21: mol_type = other DNA
gctggaagttggacacctcaa

SEQ ID NO: 39 - DNA, 22 bp, synthetic construct
  source 1..22: mol_type = other DNA
tgtagcggactactcagtaagc

SEQ ID NO: 40 - DNA, 19 bp, synthetic construct
  source 1..19: mol_type = other DNA
tggggtccttccgaggaac

SEQ ID NO: 41 - DNA, 22 bp, synthetic construct
  source 1..22: mol_type = other DNA
cagttcggctataacactggtg

SEQ ID NO: 42 - DNA, 19 bp, synthetic construct
  source 1..19: mol_type = other DNA
gcccccgacagagaagatg

SEQ ID NO: 43 - DNA, 22 bp, synthetic construct
  source 1..22: mol_type = other DNA
tgtctccagcaaagactactgt

SEQ ID NO: 44 - DNA, 23 bp, synthetic construct
  source 1..23: mol_type = other DNA
gactgtacttgacaatgttggga

SEQ ID NO: 45 - DNA, 21 bp, synthetic construct
  source 1..21: mol_type = other DNA
ctttggcatttcaggcaagca

SEQ ID NO: 46 - DNA, 23 bp, synthetic construct
  source 1..23: mol_type = other DNA
tgatgtcaggttcaaaagtgcag

SEQ ID NO: 47 - DNA, 25 bp, synthetic construct
  source 1..25: mol_type = other DNA
gacaaacctgtgagactttggctcc

SEQ ID NO: 48 - DNA, 24 bp, synthetic construct
  source 1..24: mol_type = other DNA
agtgacagcagcagttgcacagtg

SEQ ID NO: 49 - DNA, 18 bp, synthetic construct
  source 1..18: mol_type = other DNA
atttgctgtcctggcatc

SEQ ID NO: 50 - DNA, 20 bp, synthetic construct
  source 1..20: mol_type = other DNA
ccagctcattctctggaatc

SEQ ID NO: 51 - DNA, 17 bp, synthetic construct
  source 1..17: mol_type = other DNA
cgcgtttggattccgtg

SEQ ID NO: 52 - DNA, 28 bp, synthetic construct
  source 1..28: mol_type = other DNA
ccagttataatctgccttccctattatc

SEQ ID NO: 53 - DNA, 18 bp, synthetic construct
  source 1..18: mol_type = other DNA
gaggcgagggtgatagag

SEQ ID NO: 54 - DNA, 19 bp, synthetic construct
  source 1..19: mol_type = other DNA
acacacaagggtccaattc

SEQ ID NO: 55 - DNA, 20 bp, synthetic construct
  source 1..20: mol_type = other DNA
ttggtacctgtagccattcc

SEQ ID NO: 56 - DNA, 20 bp, synthetic construct
  source 1..20: mol_type = other DNA
gaggctaagaggctgctgta

SEQ ID NO: 57 - DNA, 16 bp, synthetic construct
  source 1..16: mol_type = other DNA
tcctcctatctccacc

SEQ ID NO: 58 - DNA, 16 bp, synthetic construct
  source 1..16: mol_type = other DNA
gacccagccagccata

SEQ ID NO: 59 - DNA, 20 bp, synthetic construct
  source 1..20: mol_type = other DNA
agcatacaggtcctggcatc

SEQ ID NO: 60 - DNA, 20 bp, synthetic construct
  source 1..20: mol_type = other DNA
ttcaccttcccaaagaccac

SEQ ID NO: 61 - DNA, 20 bp, synthetic construct
  source 1..20: mol_type = other DNA
tgatggctcagagcaacaag

SEQ ID NO: 62 - DNA, 20 bp, synthetic construct
  source 1..20: mol_type = other DNA
gccagggttgcactaaacat

SEQ ID NO: 63 - DNA, 20 bp, synthetic construct
  source 1..20: mol_type = other DNA
tccaagagctcggttgctat

SEQ ID NO: 64 - DNA, 20 bp, synthetic construct
  source 1..20: mol_type = other DNA
agttggggaaggtcagtgtg

SEQ ID NO: 65 - DNA, 19 bp, synthetic construct
  source 1..19: mol_type = other DNA
tcaaggcaggacagtaccc

SEQ ID NO: 66 - DNA, 23 bp, synthetic construct
  source 1..23: mol_type = other DNA
tgtatttctcccattcgcctaga

SEQ ID NO: 67 - DNA, 20 bp, synthetic construct
  source 1..20: mol_type = other DNA
gagataccccgggagttgat

SEQ ID NO: 68 - DNA, 20 bp, synthetic construct
  source 1..20: mol_type = other DNA
tcttgaaaactggaggaatg

SEQ ID NO: 69 - DNA, 21 bp, synthetic construct
  source 1..21: mol_type = other DNA
gcagaagatactgtcaccacc

SEQ ID NO: 70 - DNA, 23 bp, synthetic construct
  source 1..23: mol_type = other DNA
tttggcaaatacaacccttcaga

SEQ ID NO: 71 - DNA, 20 bp, synthetic construct
  source 1..20: mol_type = other DNA
gggcagaatcatcacgaagt

SEQ ID NO: 72 - DNA, 20 bp, synthetic construct
  source 1..20: mol_type = other DNA
tggtgatgttggactcctca

SEQ ID NO: 73 - DNA, 20 bp, synthetic construct
  source 1..20: mol_type = other DNA
ttcatctgcactgccaagac

SEQ ID NO: 74 - DNA, 20 bp, synthetic construct
  source 1..20: mol_type = other DNA
tcgagttgtccacagtcagc

SEQ ID NO: 75 - DNA, 17 bp, synthetic construct
  source 1..17: mol_type = other DNA
acaacaggagaaaccca

SEQ ID NO: 76 - DNA, 21 bp, synthetic construct
  source 1..21: mol_type = other DNA
cacgtcatgggtggtttcttg

SEQ ID NO: 77 - DNA, 20 bp, synthetic construct
  source 1..20: mol_type = other DNA
gcaagtgctggagaacatca

SEQ ID NO: 78 - DNA, 20 bp, synthetic construct
  source 1..20: mol_type = other DNA
gttaacttccgcgtttgctc

SEQ ID NO: 79 - DNA, 23 bp, synthetic construct
  source 1..23: mol_type = other DNA
acaacaccacatacggatgtaac

SEQ ID NO: 80 - DNA, 22 bp, synthetic construct
  source 1..22: mol_type = other DNA
cctagcaggaggtaaaggatct

SEQ ID NO: 81 - protein, 5 aa, synthetic construct
GGGGG

(19) United States
(12) Patent Application Publication
(10) Pub. No.: US 2025/0115595 A1
(43) Pub. Date: Apr. 10, 2025

(54) INHIBITORS OF HYPOXIA-INDUCIBLE FACTORS

(71) Applicant: The Johns Hopkins University, Baltimore, MD (US)

(72) Inventors: Gregg L. Semenza, Reisterstown, MD (US); Akrit Sodhi, Cockeysville, MD (US); Shaima Salman, Baltimore, MD (US); David J. Meyers, Towson, MD (US); Yousang Hwang, Baltimore, MD (US)

(21) Appl. No.: 18/293,683

(22) PCT Filed: Aug. 9, 2022
(86) PCT No.: PCT/US2022/039883

§ 371 Date: Jan. 30, 2024

Related U.S. Application Data
(60) Provisional application No. 63/231,216, filed on Aug. 9, 2021.

(51) Int. Cl. C07D 417/14; A61K 31/427; A61K 31/433; A61K 45/06; A61P 35/00

(52) U.S. Cl. CPC C07D 417/14; A61K 31/427; A61K 31/433; A61K 45/06; A61P 35/00

(57) ABSTRACT

This disclosure features chemical entities (e.g., a compound or a pharmaceutically acceptable salt thereof) used to treat a condition or disease that is associated with hypoxia, or with increased expression of hypoxia-inducible factor HIF-1 and/or HIF-2, or with both, the method comprising administering to a subject in need of such treatment a compound of Formula (I) that is capable of inhibiting transcriptional activation mediated by HIFs. This disclosure also features compositions containing the same as well as methods of using and making the same.

### CROSS-REFERENCE TO RELATED APPLICATIONS

This application is a National Stage application under 35 U.S.C. § 371 of International Application No. PCT/US2022/039883 having an International Filing Date of Aug. 9, 2022, which claims the benefit of priority to U.S. Provisional Application No. 63/231,216, filed on Aug. 9, 2021, which are incorporated herein by reference in their entirety.

### SEQUENCE LISTING

This application contains a Sequence Listing that has been submitted electronically as an XML file named “44807-0402US1.XML”. The XML file, created on Dec. 2, 2024, is 72,398 bytes in size. The material in the XML file is hereby incorporated by reference in its entirety.

### TECHNICAL FIELD

This disclosure features inhibitors of hypoxia-inducible factor HIF-1 and/or HIF-2, as well as methods of making and using the same. In embodiments described herein, the compounds block the ability of HIFs to alter gene expression and thereby provide therapeutic benefit in hepatocellular carcinoma (HCC) and blinding eye diseases, e.g., effectively block expression of HIF-dependent genes required for tumor vascularization and immune evasion, and block human HCC tumor xenograft growth and vascularization. In certain embodiments, the compounds described herein, when administered in combination with anti-PD1 therapy, eradicate mouse HCC tumors. In certain embodiments, the compounds described herein effectively treats retinal neovascularization and vascular hyperpermeability in mouse models of ischemic retinal disease, and choroidal neovascularization in mouse models of neovascular (wet) age-related macular degeneration.

### BACKGROUND

Hypoxia Inducible Factors (HIFs). HIFs mediate changes in gene expression in response to decreased O₂ availability (hypoxia). HIFs match O₂ supply and demand so that every cell receives adequate O₂ to meet its metabolic requirements. However, the adaptive physiological responses to hypoxia, such as the production of new blood vessels (angiogenesis) that are mediated by HIFs can be coopted by disease processes; as a result, HIFs contribute to the pathogenesis of cancer and blinding eye diseases.

### Hepatocellular Carcinoma

Hepatocellular carcinoma (HCC) is the third leading cause of cancer mortality worldwide and incidence in the U.S. has tripled over the last two decades (Bray F, et. al., CA Cancer J Clin. 2018; 68(6):394-424; Choo S P, et. al., Cancer. 2016; 122(22):3430-3446; El-Serag H B., N Engl J Med. 2011; 365(12):1118-1127). At the time of diagnosis, more than two-thirds of HCC patients have advanced disease, which is often intractable to available therapies with 5-year survival of less than 15% (El-Serag H B., N Engl J Med. 2011; 365(12):1118-1127). The first approved drug for advanced HCC, which was the tyrosine kinase inhibitor (TKI) sorafenib, provided only a modest survival benefit of 2-3 months with low response rates, high toxicity that often requires dose reduction or treatment interruption, and frequent development of resistance, and was followed by several other TKIs (lenvatinib, regorafenib, cabozantinib) and ramucirumab, which is an antibody against vascular endothelial growth factor A (VEGFA) receptor 2 (Balogh J et. al., J Hepatocell Carcinoma. 2016; 3:41-53; Llovet J M et al., N Engl J Med. 2008; 359:378-390).

Immune checkpoint blockade with antibodies that target programmed death 1 (PD1) or PD1 ligand 1 (PDL1) have revolutionized the treatment of advanced melanoma, non-small cell lung cancer, and renal cell carcinoma (Brahmer J R et al., N Engl J Med. 2012; 366(26):2455-2465; Topalian S L et al., JAMA Oncol. 2019; 5(10):1411-1420; Topalian S L et al., N Engl J Med. 2012;366;2443-2454). PDL1, which is expressed on tumor and stromal cells, binds to PD1 on T cells and triggers exhaustion or apoptosis. Nivolumab, which is an anti-PD1 antibody, was granted FDA approval for HCC based on phase II trial data, but a phase III trial versus sorafenib as first line therapy did not meet its primary endpoint with respect to overall survival, and a phase III trial of pembrolizumab, another anti-PD1 antibody, also failed versus placebo as second-line HCC therapy (Lai E, et al., Crit Rev Oncol Hematol. 2021; 157:103167; Llovet J M et al., Nat Rev Dis Primers. 2021;7(1):6). A phase III trial of atezolizumab, which is an anti-PDL1 antibody, in combination with bevacizumab, an antibody against VEGFA, led to a 2.5-month improvement in progression free survival as compared to sorafenib, although more than half of patients receiving the combination therapy suffered grade 3 or 4 adverse events (Finn R S et al., N Engl J Med. 2020; 382(20):1894-1905). Most recently, the combination of anti-PD1 (nivolumab) and anti-CTLA4 (ipilimumab) antibodies was approved for treatment of HCC patients previously treated with sorafenib based on a trial involving 49 patients, with an overall response rate of 33% (Wright K, et al., Oncology. 2020;34(4):693606). Many patients may fail to respond to immune checkpoint inhibitors (such as anti-PD1 or anti-PDL1 antibody) because of the coexistence of other mechanisms of immune evasion, such as production of adenosine, which also binds to receptors on T cells to trigger apoptosis (Huang S et al., Blood. 1997; 90(4):1600-1610).

Intratumoral hypoxia is believed to be a major driving force for cancer progression (Harris A L et al., Nat Rev Cancer. 2002; 2(1):38-47; Schito L and Semenza G L, Trends Cancer. 2016; 2(12):758-770; Vaupel P et al., Antioxid Redox Signal. 2007; 9(8):1221-1235). In breast and other cancers that are accessible to direct measurement in situ, median PO₂ levels are reduced to 10 mm Hg (1.4% O₂) and increased mortality is associated with a median PO₂<10 mm Hg (Vaupel P et al., Antioxid Redox Signal. 2007; 9(8):1221-1235). In liver cancer, the median PO₂ was 6 mm Hg as compared to 30 mm Hg in normal liver tissue (McKeown S R et. al., Br J Radiol. 2014;87(1035):20130676). Analysis of orthotopic rat HCC tumors revealed that all measured PO₂ values were between 0 and 10 mm Hg, with median values of 0.2-0.8 mm Hg, as compared to 45 mm Hg in normal rat liver (Riedl C C et al., Radiology. 2008; 248:561-570). Analysis of human HCC by dynamic contrast-enhanced magnetic resonance imaging revealed that high intratumoral blood flow was associated with increased overall survival (Chen B B et al., Radiology 2016; 281:454-464), which is consistent with an association between intratumoral hypoxia and patient mortality.

Hypoxia-inducible factors (HIFs) can play critical roles in cancer progression, by activating the transcription of a large battery of genes encoding proteins that play key roles in angiogenesis (also known as tumor vascularization), glucose metabolism, invasion/metastasis, stem cell specification, and tumor immune evasion (De Heer E C, et al., J Clin Invest. 2020; 130(10):5074-5087; Samanta D and Semenza G L, Biochim Biophys Acta Rev Cancer. 2018; 1870(1):15-22; Schito L and Semenza G L, Trends Cancer. 2016; 2(12):758-770; Semenza G L, Physiology. 2021; 36(2):73-83; Xiang L and Semenza G L, Adv Cancer Res. 2019; 141:175-212). HIFs consist of an O₂-regulated HIF-1α, HIF-2α or HIF-3α subunit and a constitutively expressed HIF-1β subunit (Semenza G L. Annu Rev Pharmacol Toxicol. 2019; 59:379-403). HIF-α subunits are subjected to O₂-dependent prolyl hydroxylation, which triggers protein degradation, and O₂-dependent asparaginyl hydroxylation, which blocks coactivator recruitment. In multiple primary studies involving a range of treatment modalities, as well as a meta-analysis, HIF-1α immunohistochemistry of tumor biopsies has revealed increased expression in >60% of HCC cases and a significant association with decreased disease-free and overall survival (Cao S et al., Clin Res Hepatol Gastroenterol. 2014; 38:598-603; Osman N A et al., Tumor Biol. 2015; 36:4293-4299; Srivastava S et al., Virchows Arch. 2015; 466:541-548; Xiao H et al., BioMed Res Int. 2014;516518; Xu W et al., J Cancer Res Clin Oncol. 2014; 140:1507-1515) as well as increased risk of recurrence after radiation therapy or surgery (Wada H et al., Liver Int. 2006; 26:414-23). In breast cancer, HIFs coordinately activate the expression of multiple proteins mediating immune evasion, including CD73, the enzyme that generates adenosine; CD47, which encodes a cell surface protein that blocks phagocytosis of cancer cells by macrophages; and PDL1, which binds to PD1 on the surface of cytotoxic T lymphocytes (CTLs) and natural killer (NK) cells to trigger anergy or apoptosis (Samanta D et al., Proc Natl Acad Sci USA. 2018;115(6):E1239-E1248). HIF-1α and PDL1 are also coexpressed in human HCC (Dai X et al., Transl Oncol. 2018; 11(2):559-566).

A screen of 3,140 drugs identified acriflavine as a drug that inhibits HIF activity (by disrupting subunit dimerization) and blocks HCC tumor growth and vascularization in mouse models (Lee K. et al., Proc Natl Acad Sci USA. 2009; 106(42):17910-17915). However, acriflavine is not an optimal candidate for clinical trials due to its propensity to cause DNA damage. Recently, a compound designated PT2385 was shown to selectively block dimerization of HIF-2α with HIF-1β (Wallace E M et al., Cancer Res. 2016; 76(18):5491-5500) and is now in advanced stage clinical trials in patients with renal cell carcinoma after encouraging phase I results (Courtney K D et al., J Clin Oncol. 2018; 36(9):867-874).

### Blinding Eye Diseases

Blinding eye diseases include a number of disorders that effect vision. Age-related macular degeneration (AMD) is a leading cause of ocular dysfunction, including blindness. One form of AMD, non-exudative AMD, also known as “dry” AMD, is typically characterized by drusen accumulation within or external to the retinal pigment epithelium (RPE). Late in dry AMD, atrophy of the RPE and the overlying rod and cone photoreceptors occurs. A second form of AMD, exudative or “wet” or “neovascular” AMD, is characterized by choroidal neovascularization (CNV). Early-stage disease is frequently dry AMD while later stage disease is frequently either wet (neovascular) AMD or advanced dry (atrophic) AMD.

Neovascular or “wet” age-related macular degeneration (wet AMD) is a leading cause of severe vision loss in elderly Americans. The growth of abnormal leaky blood vessels (i.e., CNV) in these patients can lead to rapid and often irreversible vision loss. The recent introduction of therapies targeting vascular endothelial growth factor (VEGF), which is an endothelial mitogen and permeability factor, has had a remarkable impact on patients with wet AMD who previously suffered vision loss from edema, bleeding and scarring caused by CNV. Nonetheless, more than half of wet AMD patients treated with anti-VEGF therapies have an inadequate response to treatment despite monthly injections. Moreover, it is believed that chronic VEGF inhibition can lead to the promotion of geographic atrophy and glaucoma. Collectively, these observations emphasize the importance of ongoing efforts to identify other angiogenic factors that contribute to the development of CNV and that may therefore serve as additional (and perhaps safer) targets for the treatment of patients with wet AMD.

In this regard, the transcriptional activator hypoxia-inducible factor (HIF)-1 has been hypothesized to play an important role in regulating the pathologic expression of numerous angiogenic mediators (including VEGF) that together promote ocular neovascularization. In AMD, it is hypothesized that outer retinal ischemia, due to interruption of O₂ delivery from the choriocapillaris to the overlying retinal pigment epithelium (RPE), results in HIF-1α accumulation. The choriocapillaris underlying the macula becomes attenuated (and its function compromised) with aging. In patients with AMD, material known as drusen accumulates underneath and within the thickened RPE basement membrane (also known as Bruch's membrane), forming a physical barrier for O₂ diffusion and further exacerbating the impaired O₂ delivery to the overlying RPE, and the “pathological” induction of HIF-1α.

There is growing pre-clinical evidence that HIF-1 participates in the regulation of VEGF expression and the development of CNV in patients with wet AMD. Increased expression of HIF-1α has been reported in CNV membranes in wet AMD eyes. Inhibition of HIF-1 in the RPE, either pharmacologically or genetically, decreases the size of CNV lesions in mouse models.

Several multicenter randomized controlled clinical trials have also demonstrated the benefit of monthly (or bi-monthly) injections with anti-VEGF therapies to treat macular edema in patients with ischemic retinopathies (IRs), which include diabetic retinopathy, retinal vein occlusion, sickle cell retinopathy, and retinopathy of prematurity. However, similar to wet AMD, less than 50% of treated IR patients demonstrate a major improvement in vision (i.e., a gain of at least 15 letters on the ETDRS vision chart) despite monthly treatment. Similarly, use of monthly anti-VEGF therapy inhibits the progression to neovascularization (NV) in some—but not all—IR patients.

These clinical observations for the role of other HIF-regulated angiogenic mediators in these diverse IRs are supported by robust pre-clinical studies. Studies using animal models have demonstrated that expression of a constitutively-active form of the transcription factor HIF-1α was sufficient to promote ocular NV in vivo, while expression of VEGF alone was not sufficient to mediate this effect. These results potentially point to additional HIF-regulated angiogenic factor(s) in the promotion of pathological NV in the eye.

A small molecule inhibitor, PT2385 that has selectively binds HIF-2α—but not HIF-1α—to prevent it from binding to HIF-18, has shown promise in preclinical studies, and is currently under investigation for the treatment of patients with renal cell carcinoma (Courtney, K D, et al, J Clin Oncol 2018 Mar. 20;36(9):867-874). However, it has recently demonstrated that expression of both HIF-1α and HIF-2α is increased in ocular neovascular disease and that both HIFs participate in promoting VEGF expression in IRs, but that HIF-1 alone is sufficient to promote retinal NV in mice. Thus, therapies targeting only HIF-2 may not be sufficient to prevent ocular neovascularization.

### SUMMARY

This disclosure features chemical entities (e.g., a compound or a pharmaceutically acceptable salt thereof) that inhibit HIF-1 and/or HIF-2 (e.g., a chemical entity that inhibits transcriptional activation mediated by HIFs, thereby eliminating HIF-dependent gene expression and subsequent physiological responses). Said chemical entities are useful, e.g., for treating a condition, disease or disorder in which increased (e.g., excessive) HIF-1 and/or HIF-2 activity contributes to the pathology and/or symptoms and/or progression of the condition, disease or disorder. Two major categories of human disease in which there is strong scientific evidence that HIFs play a major role in disease pathogenesis are cancer, e.g., hepatocellular cancer (HCC), and blinding eye diseases, e.g., wet or neovascular AMD, ischemic retinopathies (IRs) (that include diabetic retinopathy, retinal vein occlusions, sickle cell retinopathy, and retinopathy of prematurity), corneal neovascularization, and neovascular glaucoma. The chemical entities disclosed herein are shown to block HIF-dependent gene expression and to provide therapeutic benefit in mouse models of HCC, AMD, and IRs. This disclosure also features compositions containing the same as well as methods of using and making the same. Said methods include, but are not limited to, methods of treating a condition, disease or disorder that is associated with hypoxia, or with increased expression of hypoxia-inducible factor HIF-1α and/or HIF-2α, or with both. The methods include administering to a subject in need of such treatment a compound that inhibits HIF-1 and/or HIF-2; e.g., a compound of Formula (I). In embodiments, the compound that inhibits HIF-1 and/or HIF-2 inhibits transcriptional activation mediated by HIFs. In embodiments, the compounds described herein inhibit HIF-1 and HIF-2.

In one aspect, this disclosure features compounds of Formula (I):

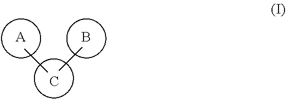

or a pharmaceutically acceptable salt thereof, wherein:
Ring A is heteroaryl including from 8-10 ring atoms, wherein from 1-4 ring atoms are heteroatoms each independently selected from the group consisting of: N, N(R^{a}), O, and S(O)₀₋₂, which is optionally substituted with from 1-4 X^{a};
Ring B is heteroaryl including from 8-10 ring atoms, wherein from 1-4 ring atoms are heteroatoms each independently selected from the group consisting of: N, N(R^{a}), O, and S(O)₀₋₂, which is optionally substituted with from 1-4 X^{b};
Ring C is heteroaryl including from 5-6 ring atoms, wherein from 1-4 ring atoms are heteroatoms each independently selected from the group consisting of: N, N(R^{a}), O, and S(O)₀₋₂, which is optionally substituted with from 1-2 R^{b};
each X^{a} and X^{b} is independently selected from the group consisting of: halo, C₁₋₆ alkyl, C₁₋₆ haloalkyl, C₂₋₆ alkenyl, C₂₋₆ alkynyl, —NO₂, cyano, C₁₋₆ alkoxy, C₁₋₆ haloalkoxy, SO₂(C₁₋₆ alkyl), heterocyclyl, heteroaryl, C₃₋₆ cycloalkyl, NR¹R², C(O)R³, and C(O)NR¹R²;
each occurrence of R^{a} is independently selected from the group consisting of: H, C₁₋₄ alkyl, —C(═O)R³, —C(═O)OR³, —C(═O)NR¹R², and —SO₂R⁴;
each occurrence of R^{b} is independently selected from the group consisting of: H, C₁₋₄ alkyl, C₁₋₄haloalkyl, halo, and cyano;
each occurrence of R¹ and R² is independently selected from the group consisting of: H, C₁₋₄ alkyl, optionally substituted C₃₋₆ cycloalkyl, optionally substituted phenyl, optionally substituted benzyl, C(═O)R⁵, C(═O)OR⁵, and C(═O)NR⁵R⁶; or
a pair of R¹ and R² on the same nitrogen atom, taken together with said nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of: N, N(R^{a}), O, and S;
each occurrence of R³ is independently selected from the group consisting of: H, C₁₋₄ alkyl, C₁₋₄haloalkyl, optionally substituted C₃₋₆cycloalkyl, and optionally substituted phenyl;
each occurrence of R⁴ is independently selected from the group consisting of: C₁₋₄ alkyl, C₁₋₄haloalkyl, optionally substituted C₃₋₆ cycloalkyl, optionally substituted phenyl; and optionally substituted benzyl; and
each occurrence of R⁵ and R⁶ is independently selected from the group consisting of: H, C₁₋₄ alkyl.

In another aspect, this disclosure features pharmaceutical compositions that include one or more compounds of Formula (I), and one or more pharmaceutically acceptable carriers.

In another aspect, this disclosure features methods for inhibiting HIF-1 and/or HIF-2 activity that include contacting a cell with an effective amount of a compound of Formula (I) (including pharmaceutically acceptable salts thereof). The cell may be contacted in vitro or in vivo with the compound of Formula (I). The cell also may be contacted with a second therapeutic agent.

In another aspect, this disclosure features methods that include administering to a subject having, or suspected of having, a condition mediated by hypoxia a compound of Formula (I) (including pharmaceutically acceptable salts thereof) and/or pharmaceutical compositions thereof. In certain embodiments, the compound of Formula (I) inhibits transcriptional activation mediated by HIFs. In certain embodiments, the condition, disease or disorder is a cancer, e.g., hepatocellular cancer. In other embodiments, the condition, disease or disorder is blinding eye disease, e.g., wet or neovascular AMD, ischemic retinopathies (IRs), such as diabetic retinopathy, retinal vein occlusions, sickle cell retinopathy, and retinopathy of prematurity, corneal neovascularization, and neovascular glaucoma.

In another aspect, the present disclosure provides the use of the compounds or the pharmaceutical compositions disclosed herein in an amount effective for treating a condition, disease or disorder associated with hypoxia, or with increased expression of hypoxia-inducible factor (HIF)-1α and/or HIF-2α, or with both, the method comprising administering to a subject in need of such treatment a compound of Formula (I) that is capable of inhibiting transcriptional activation mediated by HIFs. The contemplated uses include of the use of a compound of Formula (I) in the manufacture of a medicament for treating said condition, disease or disorder. In certain embodiments, the condition, disease or disorder is cancer, e.g., hepatocellular cancer. In other embodiments, the condition, disease or disorder is blinding eye disease, e.g., wet or neovascular AMD, ischemic retinopathies (IRs), such as diabetic retinopathy, retinal vein occlusions, sickle cell retinopathy, and retinopathy of prematurity, corneal neovascularization, and neovascular glaucoma.

Accordingly, in one aspect, the present disclosure provides a method for treating a condition, disease or disorder associated with hypoxia, or with increased expression of hypoxia-inducible factor (HIF)-1α and/or HIF-2α, comprising administering to the subject a compound of Formula (I), a pharmaceutically acceptable salt thereof, or a pharmaceutical composition thereof. In some embodiments, the method further comprises administering to the subject at least one additional therapeutic agent. In certain embodiments, the condition, disease or disorder is cancer, e.g., hepatocellular cancer. In other embodiments, the condition, disease or disorder is blinding eye disease, e.g., wet or neovascular AMD, ischemic retinopathies (IRs), such as diabetic retinopathy, retinal vein occlusions, sickle cell retinopathy, and retinopathy of prematurity, corneal neovascularization, and neovascular glaucoma.

In another aspect, this disclosure provides the use of the compounds or the pharmaceutical compositions disclosed herein in an amount effective for treating a hyperproliferative disease (e.g., cancer) in a subject comprising administering to the subject a pharmaceutical composition comprising a compound of Formula (I). In some embodiments, the use comprises administering to the subject at least one additional therapeutic agent. In certain embodiments, the hyperproliferative disease (e.g., cancer) is hepatocellular cancer.

In another aspect, this disclosure features a method for treating cancer or a hyperproliferative disease in a subject comprising administering to the subject a compound of Formula (I), a pharmaceutically acceptable salt thereof, or a pharmaceutical composition thereof. In certain of these embodiments, the method further comprises administering to the subject at least one additional therapeutic agent. In certain embodiments, the hyperproliferative disease (e.g., cancer) is hepatocellular cancer.

In one aspect, this disclosure features methods of treating hepatocellular cancer, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof.

In another aspect, the present disclosure provides a method for modulating angiogenesis in a subject, comprising administering to the subject a compound of Formula (I), a pharmaceutically acceptable salt thereof, or a pharmaceutical composition thereof. In certain of these embodiments, the method further comprises administering to the subject at least one additional therapeutic agent.

In one aspect, this disclosure features methods of treating an ocular disease, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating a blinding eye disease, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating wet or neovascular AMD, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating ischemic retinopathies (IRs), which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In certain embodiments, the IR is diabetic retinopathy, retinal vein occlusions, sickle cell retinopathy, and retinopathy of prematurity). In some embodiments, the administering is effected orally or intra-vascularly, or intraocularly, or periocularly, or to the ocular surface.

In one aspect, this disclosure features methods of treating corneal neovascularization, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating neovascular glaucoma, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In another aspect, this disclosure features compounds of Formula (I) and pharmaceutical compositions thereof capable of reducing inflammation by inhibiting HIF-1 activation. In particular, certain embodiments of the disclosed compounds and pharmaceutical compositions thereof have an anti-inflammatory effect. A compound of Formula (I) or a pharmaceutical composition thereof may be formulated for use in medicine, and a therapeutically effective amount of the compound of Formula (I) or pharmaceutical composition thereof may be administered to a subject having, or suspected of having, a condition that produces an inflammatory response mediated by hypoxia. Exemplary conditions that produce an inflammatory response mediated by hypoxia include without limitation malignant tumors, intestinal inflammation (e.g., inflammatory bowel disease), lung inflammation (e.g., resulting from acute lung injury), ischemia, atherosclerosis, myocardial infarction, rheumatoid arthritis, and wound healing.

In one aspect, this disclosure features methods of treating a hyperproliferative disease, such as cancer, which include administering to a subject in need of such treatment an effective amount of an inhibitor of HIF-1 and/or HIF-2 and a chemotherapeutic immunomodulatory agent, such as an immune checkpoint inhibitor.

In another aspect, this disclosure describes subject selection criteria, such as a definitive diagnosis of an inflammatory condition mediated by hypoxia based on, for example, clinical signs and symptoms and/or laboratory evidence of inflammation. An example of such a subject would be a person having an elevated C-reactive protein level.

In another aspect, this disclosure provides the use of the compounds or the pharmaceutical compositions disclosed herein in an amount effective for treating ocular diseases including but not limited to Diabetic Retinopathy, Retinal Vein Occlusion, Sickle Cell Retinopathy, Retinopathy of Prematurity, Age-related Macular Degeneration, Corneal Neovascualrization, and Neovascular Glaucoma.

In another aspect, this disclosure provides the use of the compounds or the pharmaceutical compositions disclosed herein in an amount effective for treatment or prevention of Von Hippel-Lindau disease, or any other disease where HIF-1, HIF-2 or hypoxia response is identified as a potential mechanism for therapeutic targeting.

In certain of the foregoing embodiments, the methods further include administering to the subject one or more additional therapeutic agents. For example, methods of treating cancer as described herein can further include administering an immune checkpoint inhibitor or any other standard-of-care anti-cancer agent. As another example, methods of treating an ocular disease (e.g., a blinding eye disease) as described herein can further include administering an anti-vascular endothelial growth factor (VEGF) agent, an angiotensin-converting enzyme (ACE) inhibitor, a peroxisome proliferator-activated receptor (PPAR)-gamma agonist, a renin inhibitor, a steroid, an agent that modulates autophagy, semapimod, a MIF inhibitor, a CCR2 inhibitor, CKR-2B, a 2-thioimidazole, CAS 445479-97-0, CCX140, clodronate, a clodonate-liposome preparation and gadolinium chloride.

Embodiments can include one or more of the following advantages. In some embodiments, the compounds and methods described herein can increase a subject's response rate to, and/or reduce severity of adverse effects associated with, immune checkpoint inhibitor therapy. While not wishing to be bound by theory, it is believed that lower response rates to immune checkpoint inhibitor therapy across all human cancers is due in part to the multiplicity of molecular mechanisms (many of which are HIF-regulated) by which cancer cells evade the immune system (Semenza G L, Physiology. 2021; 36(2):73-83). It is believed that the broad effect of HIF inhibitors can provide a route to reduce the expression of a large battery of genes mediating immune evasion (FIG. 16) and thereby improve the therapeutic response to immune checkpoint blockade. Again, while not wishing to be bound by theory, it is believed that the compounds and methods described herein can be beneficial in the treatment of ocular diseases, e.g., blinding eye diseases. It is believed that additional HIF-regulated angiogenic factor(s) are involved in the promotion of pathological NV in many ocular neovascular diseases. Inhibition of HIF-1, which results in a reduction of a broad spectrum of angiogenic mediators back to physiologic levels, could therefore potentially be safer, but equally as effective as complete neutralization of VEGF. Additionally, the compounds and methods described herein can reduce the likelihood of a subject's experiencing adverse side effects associated with chronic VEGF inhibition (e.g., retinal atrophy, e.g., potentially leading to (permanent) loss of vision; e.g., elevated intraocular pressure, e.g., potentially leading to glaucoma).

In any of the foregoing embodiments, the inhibitor inhibits of HIF-1 and HIF-2 (e.g., a compound of Formula (I)).

### DESCRIPTION OF DRAWINGS

FIG. 1 depicts cell-based screening assay for HIF inhibitors. (A) Luciferase reporters, (B) Effect of 33-063 on hypoxia-induced luciferase activity, (C). Chemical structure of six compounds and their calculated IC₅₀ values.

FIG. 2 depicts the chemical structures of HIF inhibitors with IC₅₀<3.3 μM.

FIG. 3 depicts analysis of HIF target gene expression.

FIG. 4 depicts effect of HIF inhibitors on reporter gene transactivation, HIF protein expression, and HIF heterodimerization. (A) Fluc/Rluc for Hep3B co-transfected with p2.1 and pSVR, (B-C) Hep3B cells were exposed to O₂ for 4 hours (B), or for 8 hours or 24 hours (C).

FIG. 5 depicts effect of HIF inhibitors (32-134D, 33-063, and 12-143) on HIF-1α transactivation domain function. (A) (SEQ ID NO: 81) Hep3B cells were co-transfected with pG5E1bFLuc, which contains five Gal4 binding sites and encodes Fluc, and an expression vector encoding a fusion protein consisting of the Gal4 DNA-binding domain fused to HIF-1α transactivation domain (TAD) sequences. (B) HIF-1α TAD contains N-terminal (N-TAD) and C-terminal (C-TAD) activation subdomains and an inhibitory domain (ID); 5 GAL4 fusion proteins, containing the indicated HIF-1α residues, were tested. (C) Co-transfected cells exposed to 20% O₂ (white bar) or 1% O₂ (black bar) for 24 h in the presence of vehicle (Veh; DMSO); or were treated with the indicated HIF inhibitor (32-134D, 33-063 or 12-143) at a concentration of 10 μM at 20% O₂ (white bars) or 1% O (red bars).

FIG. 6 depicts dose response of compound 32-134D on Hep3B tumor xenograft growth (tumor volume) in nude mice, presented either as the growth of individual tumors (A) or the mean tumor growth (B), and the lack of any effect of 32-134D on body weight of the mice (C).

FIG. 7 depicts effect of 32-134D (administered at a dose of 40 mg/kg/day) on Hep3B tumor xenograft growth in nude mice, presented either as the growth of individual tumors (A) or the mean tumor growth (B), and the lack of any effect of 32-134D on body weight of the mice (C). The appearance (D) and weight (E) of the excised tumors is also shown.

FIG. 8 depicts effect of 32-134D on Hep3B tumor angiogenesis and immune evasion. (A) Total RNA isolated from tumor tissue was analyzed and demonstrated decreased expression of mRNAs encoding proteins that mediate angiogenesis or immune evasion in tumors from 32-134D-treated mice. (B) ELISA showing decreased expression of pro-angiogenic proteins in 32-134D-treated mice. (C) Tumor sections were treated with CD31 antibody, demonstrating decreased vascularization of tumors from 32-134D-treated mice.

FIG. 9 depicts effect of HIF inhibitors (32-134D, 33-063) on hypoxia-induced expression of angiogenic factor mRNAs in Hepa1-6 cells, as compared to the effect of PT-2385 or vehicle (bar at far left).

FIG. 10 depicts effect of 32-134D at a concentration of 5 μM on hypoxia-induced expression of mRNAs encoding proteins that mediate immune evasion in Hepa1-6 cells.

FIG. 11 depicts effect of anti-PD1 and 32-134D on Hepa1-6 tumor growth in syngeneic (immunocompetent) mice, demonstrating efficacy of 32-134D and superior efficacy of combination therapy compared to anti-PD1 alone.

FIG. 12 depicts effect of 32-134D on the tumor immune microenvironment, demonstrating increased numbers of activated T cells and natural killer (NK) cells, which mediate killing of cancer cells; and decreased numbers of tumor-associated macrophages (TAMs) and myeloid-derived suppressor cells (MDSCs), which mediate immune evasion.

FIG. 13 depicts effect of 32-134D on intratumoral gene expression, demonstrating decreased expression of mRNAs encoding angiogenic factors (A) and proteins mediating immune evasion (C top right and D), and increased expression of mRNAs encoding proteins that mediate anti-tumor immunity (C bottom left).

FIG. 14 depicts effect of 32-134D on intratumoral expression of mRNAs encoding cytokines and chemokines.

FIG. 15 depicts effect of 32-134D on intratumoral expression of immunoregulatory proteins as determined by ELISA.

FIG. 16 depicts effect of 32-134D on gene expression consistent with therapeutic benefit.

FIG. 17 depicts safety; fundus photos and fluorescein angiographic images of the retina of C57BL/6 mice after treatment (IP dosing) with 32-134D.

FIG. 18 depicts safety; ERG measurements taken in C57BL/6 mice following daily IP injections with 32-134D.

FIG. 19 depicts safety; images of various tissues from mice treated (IP dosing) with 32-134D compared to vehicle control.

FIG. 20 depicts efficacy; decreased expression of Vegf and Angpt14 (CNV model of wet-type age-related macular degeneration) after treatment (IP dosing) 32-134D in C57BL/6 mice.

FIG. 21 depicts efficacy; reduction of choroidal neovascular lesions (CNV model for wet AMD) following treatment (IP dosing) 32-134D in C57BL/6 mice.

FIG. 22 depicts efficacy; reduced vascular leakage (STZ model of diabetic edema) following treatment (IP dosing) 32-134D in C57BL/6 mice.

FIG. 23 depicts efficacy; reduction of macular edema (I/R model of vascular permeability associated with retinal vein occlusion) following treatment (IP dosing) 32-134D in C57BL/6 mice.

FIG. 24 depicts efficacy; decreased neovascularization (OIR model for ischemic retinal neovascularization) following treatment (IP dosing) 32-134D in C57BL/6 mice pups.

FIG. 25 depicts efficacy; decreased lesion area (Laser CNV model of neovascular AMD) following intraocular administration of 32-134D in C57BL/6 mice.

FIG. 26 depicts efficacy; decreased retinal neovascularization (OIR model for ischemic retinal neovascularization) following intraocular administration of 32-134D in C57BL/6 mice pups.

FIG. 27 depicts inhibition of HIF-1α and HIF-2α accumulation by 32-134D in an immortalized human retinal Müller glial cell line (MIO-M1) cultured under hypoxic conditions in the presence of 1 or 10 μM 32-134D for 4 hours (A) or in the presence of 10 μM 32-134D from 1 to 24 hours (B).

FIG. 28 depicts mRNA expression in MIO-M1 cells cultured under hypoxic conditions in the presence of vehicle or 32-134D (10 μM): (A) vascular endothelial cell growth factor (Vegf) (A) and (B) angiopoietin-like 4 (Angpt14).

FIG. 29 depicts inhibition of HIF-1α and HIF-2α accumulation in MIO-M1 cells cultured under hypoxic conditions for 4 hours in the presence of MG-132 and 32-134D (10 μM) (A), or inhibition of HIF-1α accumulation in MIO-M1 cells cultured under hypoxic conditions for 4 hours in the presence of MG-132 or bortezomib (10 μM), or bafilomycin (10 nM) (B).

FIG. 30 depicts inhibition of HIF-1α and HIF-2α accumulation by 32-134D (10 μM) in human umbilical vein endothelial cells (HUVECs) cultured under hypoxic conditions for 4 hours.

FIG. 31 depicts expression of the following mRNAs in HUVECs cultured under hypoxic conditions for 4 hours in the presence of vehicle or 32-134D (1 or 10 μM): (A) Vegf and Angpt14; (B) angiopoietin 2 (Angpt2) and vascular endothelial protein tyrosine phosphatase (Veptp); and (C) plasminogen activator inhibitor 1 (Pai1).

FIG. 32 depicts (A) Brightfield image of 120-day old (D120) retinal organoid derived from human-induced pluripotent stem cells (hiPSCs); (B) hematoxilin and eosin staining of D120 hiPSC-derived retinal organoid; (C and D) representative immunofluorescence images of D120 hiPSC-derived retinal organoid demonstrating staining for Pax6 and recoverin (REC; C) and CRALBP (D) in inner and outer retinal cells. Scale bar=25 μm.

FIG. 33 depicts inhibition of HIF-1α and HIF-2α accumulation in D120 hiPSC-derived retinal organoids cultured under hypoxic conditions for 12 hours in the presence of 32-134D (1, 10, or 100 μM).

FIG. 34 depicts expression of Vegf (A) and Angpt14 (B) mRNA in D120 hiPSC-derived retinal organoids cultured under hypoxic conditions for 12 hours in the presence of 32-134D (1 or 10 μM).

FIG. 35 depicts HIF-1α (left) and HIF-2α (right) accumulation 24 hours after a single intraperitoneal injection of 32-134D (20 mg/kg) in OIR postnatal day 13 (P13) and P14 mice, respectively.

FIG. 36 depicts mRNA expression in P17 OIR mice treated with five consecutive (P12-P16) IP injections with 20 mg/kg 32-134D of vasoactive mediators Vegf, Angpt14, platelet-derived growth factor (Pdgf), and erythropoietin (Epo) (A), including those expressed specifically by endothelial cell [Angpt2, Veptp, Pai1, and VEGF receptor 2 (Kdr)](B).

FIG. 37 depicts (A) HIF-1α and HIF-2α expression or expression of Vegf and Angpt14; (B) or Angpt2 and Veptp (C) mRNA in neurosensory retinas from streptozotocin (STZ)-induced diabetic mice that were hyperglycemic (serum glucose greater than 250 mg/dl) for 6 months prior to treatment with a single intraperitoneal injection with 32-134D (40 or 80 mg/kg) 24 hours prior to sacrifice.

FIG. 38 depicts electroretinograms (ERGs) taken weekly of C57BL/6 mice following a single intraocular administration of increasing doses of 32-134D (70, 140, 210, or 350 ng) compared to vehicle control (0).

FIG. 39 depicts hematoxylin and eosin staining of retinal sections 35 days following a single intraocular injection with 210 ng or 350 ng of 32-134D. Inset demonstrates high magnificent images of the ganglion cell layer (GCL). Scale bar=200 μm

FIG. 40 depicts immunostaining for the ganglion cell marker RNA Binding Protein, mRNA Processing Factor (RBPMS) (A) in RGCs in retinal flat mounts 35 days after a single intraocular injection with 210 ng or 350 ng of 32-134D; (B) and quantitation of RGCs observed in A.

FIG. 41 depicts HIF-1α accumulation in MIO-M1 cells cultured under hypoxic conditions for 4 hours in the presence of increasing concentrations of 32-134D.

FIG. 42 depicts (A) quantitation of immunoblots for HIF-1α accumulation in 32-134D-treated MIO-M1 cells cultured under hypoxic conditions for 4 hours (from FIG. 41) demonstrating the half maximal inhibitory concentration (IC₅₀) of 3.5 μM; and (B) concentration-time profiles of 32-134D in mice treated with a single intraocular injection with 70 ng of 32-134D. The concentration of 32-134D in the neurosensory retina exceeded the calculated in vitro IC₅₀ of 3.5 μM for 5.25 days.

FIG. 43 depicts HIF-1α and HIF-2α expression 24 hours following a single intraocular injection with 70 ng of 32-134D in P13 and P14 OIR mice, respectively.

FIG. 44 depicts expression of mRNA of HIF-regulated genes Vegf, Angpt14, Kdr, Epo, and Pai1 at P17 in OIR mice treated with a single intraocular injection with 70 ng of 32-134D at P12.

FIG. 45 depicts images of vascular hyper-permeability as demonstrated by leakage of intravascular Evans blue dye in STZ mice that were hyperglycemic for 6 months before treatment with 70 ng of 32-134D 5 days prior to sacrifice (left); and quantitation of vascular hyper-permeability (right).

FIG. 46 depicts weekly ERG of C57BL/6 mice following a single intraocular administration of 500 ng or 1,000 ng doses of 32-134D compared to vehicle control (0).

FIG. 47 depicts concentration of 32-134D in the neurosensory retina after a single intraocular injection with 280 ng. The concentration of 32-134D exceeded the in vitro IC₅₀ of 3.5 μM for at least 11.7 days after the 280 ng dose.

### DETAILED DESCRIPTION

This disclosure features chemical entities (e.g., a compound or a pharmaceutically acceptable salt thereof) that inhibit HIF-1 and/or HIF-2 (e.g., a chemical entity that inhibits transcriptional activation mediated by HIFs. Said chemical entities are useful, e.g., for treating a condition, disease or disorder in which increased (e.g., excessive) HIF-1 and/or HIF-2 activity contributes to the pathology and/or symptoms and/or progression of the condition, disease or disorder, e.g., a cancer such as hepatocellular cancer, or a blinding eye disease, such as wet (neovascular) AMD, or one of the ischemic retinopathies (IRs) that include diabetic retinopathy, retinal vein occlusion, sickle cell retinopathy, and retinopathy of prematurity) in a subject (e.g., a human). This disclosure also features compositions containing the same as well as methods of using and making the same. Said methods include, but are not limited to, methods of treating a condition, disease or disorder that is associated with hypoxia, or with increased expression of hypoxia-inducible factor HIF-1α and/or HIF-2α, or with both. The methods include administering to a subject in need of such treatment a compound that inhibits HIF-1 and/or HIF-2; e.g., a compound of Formula (I). In embodiments, the compound that inhibits HIF-1 and/or HIF-2 inhibits transcriptional activation mediated by HIFs. In embodiments, the compounds described herein inhibits HIF-1 and HIF-2.

### Compounds of Formula (I)

In one aspect, this disclosure features compounds of Formula (I):

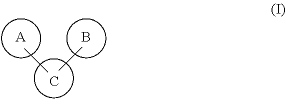

or a pharmaceutically acceptable salt thereof, wherein:
Ring A is heteroaryl including from 8-10 ring atoms, wherein from 1-4 ring atoms are heteroatoms each independently selected from the group consisting of: N, N(R^{a}), O, and S(O)₀₋₂, which is optionally substituted with from 1-4 X^{a};
Ring B is heteroaryl including from 8-10 ring atoms, wherein from 1-4 ring atoms are heteroatoms each independently selected from the group consisting of: N, N(R^{a}), O, and S(O)₀₋₂, which is optionally substituted with from 1-4 X^{b};
Ring C is heteroaryl including from 5-6 ring atoms, wherein from 1-4 ring atoms are heteroatoms each independently selected from the group consisting of: N, N(R^{a}), O, and S(O)₀₋₂, which is optionally substituted with from 1-2 R^{b};
each X^{a} and X^{b} is independently selected from the group consisting of: halo, C₁₋₆ alkyl, C₁₋₆ haloalkyl, C₂₋₆ alkenyl, C₂₋₆ alkynyl, —NO₂, cyano, C₁₋₆ alkoxy, C₁₋₆ haloalkoxy, SO₂(C₁₋₆ alkyl), heterocyclyl, heteroaryl, C₃₋₆ cycloalkyl, NR¹R², C(O)R³, and C(O)NR¹R²;
each occurrence of R^{a} is independently selected from the group consisting of: H, C₁₋₄ alkyl, —C(═O)R³, —C(═O)OR³, —C(═O)NR¹R², and —SO₂R⁴;
each occurrence of R^{b} is independently selected from the group consisting of: H, C₁₋₄ alkyl, C₁₋₄haloalkyl, halo, and cyano;
each occurrence of R¹ and R² is independently selected from the group consisting of: H, C₁₋₄ alkyl, optionally substituted C₃₋₆ cycloalkyl, optionally substituted phenyl, optionally substituted benzyl, C(═O)R⁵, C(═O)OR⁵, and C(═O)NR⁵R⁶; or
a pair of R¹ and R² on the same nitrogen atom, taken together with said nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of: N, N(R^{a}), O, and S;
each occurrence of R³ is independently selected from the group consisting of: H, C₁₋₄ alkyl, C₁₋₄haloalkyl, optionally substituted C₃₋₆cycloalkyl, and optionally substituted phenyl;
each occurrence of R⁴ is independently selected from the group consisting of: C₁₋₄ alkyl, C₁₋₄haloalkyl, optionally substituted C₃₋₆ cycloalkyl, optionally substituted phenyl; and optionally substituted benzyl; and
each occurrence of R⁵ and R⁶ is independently selected from the group consisting of: H, C₁₋₄ alkyl.

In some embodiments, the compounds and methods described herein exclude one or more compounds disclosed in WO 2018/060367 [compounds 2-25], WO 1998/018466 [compounds I(a)-I(i), II(j), III(k), III(l), IV(m), V(n)-V(p), VI(p), VI(q) VII(r), VIII(s), IX(t), X(u), and XI(v)], U.S. Pat. No. 5,428,175A[compound HS-1], U.S. Pat. No. 5,290,777A [compounds I(a)-I(h), II(j)-II(l), III(m), III(n), and IV(o)], U.S. Pat. No. 4,970,226(A) [Nortopentin A-D], WO 1991/004975) [Nortopentin A-D], WO 2001/094310 [compounds 2, 3, 16, 23, and 26], CN 109418267 A [Nortopentin A-D, and compounds Ia-1 to Ia-8, Ib-1 to Ib-8, Ic-1 and Ic-2], WO 2015/173321 [2,4-bis(benzo[d][1,3]dioxol-5-yl)-1H-imidazole], and JP2009120589A, each of which is incorporated by reference in its entirety.

As used herein, the term “halo” refers to fluoro (F), chloro (Cl), bromo (Br), or iodo (I).

As used herein, the term “alkyl” refers to a hydrocarbon chain that may be a straight chain or branched chain, containing the indicated number of carbon atoms. For example, C₁₋₆ indicates that the group may have from 1 to 6 (inclusive) carbon atoms in it. Non-limiting examples include methyl, ethyl, iso-propyl, tert-butyl, n-hexyl.

As used herein, the term “haloalkyl” refers to an alkyl, in which one or more hydrogen atoms is/are replaced with an independently selected halo.

As used herein, the term “alkoxy” refers to an —O-alkyl radical (e.g., —OCH₃). Accordingly, the term “haloalkoxy” refers to an —O-haloalkyl radical (e.g., —OCF₃).

As used herein, the term “alkylene” refers to a divalent alkyl (e.g., —CH₂—).

As used herein, the term “heterocyclyl” refers to a mono-, bi-, tri-, or polycyclic nonaromatic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S(O)₀₋₂ (e.g., carbon atoms and 1-3, 1-6, or 1-9 heteroatoms of N, O, or S(O)₀₋₂ if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “ary” refers to monovalent aromatic carbocyclic group of, unless specified otherwise, from 6 to 15 carbon atoms having a single ring (e.g., phenyl) or multiple condensed rings which condensed rings may or may not be aromatic (e.g., benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic. Unless otherwise specified, an aryl group may be substituted or unsubstituted.

As used herein, the term “alkenyl” refers to a radical of a straight-chain or branched hydrocarbon group having one or more carbon-carbon double bonds, and no triple bonds. In some embodiments, an alkenyl group has 2 to 6 carbon atoms (“C₂-6 alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C₂₋₄ alkenyl groups include ethenyl (C2), 1-propenyl (C3), 2-propenyl (C3), 1-butenyl (C4), 2-butenyl (C4), butadienyl (C4), and the like. Examples of C2-C6 alkenyl groups include the aforementioned C₂₋₄ alkenyl groups as well as pentenyl (C5), pentadienyl (C5), hexenyl (C6), and the like. Each instance of an alkenyl group may be independently optionally substituted, e.g., unsubstituted (an “unsubstituted alkenyl”) or substituted (a “substituted alkenyl”) with one or more substituents, e.g., from 1 to 5 substituents, 1 to 3 substituents, or 1 substituent. In certain embodiments, the alkenyl group is unsubstituted C₂₋₆ alkenyl. In certain embodiments, the alkenyl group is substituted C₂₋₆ alkenyl.

As used herein, the term “alkynyl” refers to a radical of a straight-chain or branched hydrocarbon group having one or more carbon-carbon triple bonds, and no double bonds. In some embodiments, an alkynyl group has 2 to 6 carbon atoms (“C₂₋₆ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C2 alkynyl”). The one or more carbon-carbon triple bonds can be internal (such as in 2-butynyl) or terminal (such as in 1-butynyl). Examples of C₂₋₄ alkynyl groups include ethynyl (C2), 1-propynyl (C3), 2-propynyl (C3), 1-butynyl (C4), 2-butynyl (C4), and the like. Examples of C2-C6 alkenyl groups include the aforementioned C₂₋₄ alkenyl groups as well as pentynyl (C5), hexynyl (C6), and the like. Each instance of an alkynyl group may be independently optionally substituted, e.g., unsubstituted (an “unsubstituted alkynyl”) or substituted (a “substituted alkynyl”) with one or more substituents, e.g., from 1 to 5 substituents, 1 to 3 substituents, or 1 substituent. In certain embodiments, the alkenyl group is unsubstituted C₂₋₆ alkynyl. In certain embodiments, the alkenyl group is substituted C₂₋₆ alkynyl.

As used herein, the term “cycloalkyl” refers to a radical of a non-aromatic cyclic hydrocarbon group with zero heteroatoms in the non-aromatic ring system. In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). Exemplary C3-C6 cycloalkyl groups include, without limitation, cyclopropyl (C3), cyclopropenyl (C3), cyclobutyl (C₄), cyclobutenyl (C4), cyclopentyl (C5), cyclopentenyl (C5), cyclohexyl (C6), cyclohexenyl (C6), cyclohexadienyl (C6), and the like. In certain embodiments, the cycloalkyl group is either monocyclic (“monocyclic cycloalkyl”) or contain a fused, bridged, or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, e.g., unsubstituted (an “unsubstituted cycloalkyl”) or substituted (a “substituted cycloalkyl”) with one or more substituents. In certain embodiments, the cycloalkyl group is unsubstituted C₃₋₆ cycloalkyl. In certain embodiments, the cycloalkyl group is a substituted C₃₋₆ cycloalkyl.

Included within the compounds of the present disclosure are the tautomeric forms of the disclosed compounds, isomeric forms including diastereoisomers, and the pharmaceutically acceptable salts thereof.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)-, and individual isomers are encompassed within the scope of the disclosure. The compounds of the present disclosure do not include those which are known in art to be too unstable to synthesize and/or isolate. The disclosure is meant to include compounds in racemic and optically pure forms. Optically active (R)- and (S)-, isomers may be prepared using chiral synthons or chiral reagents as disclosed herein, or resolved using conventional techniques. When the compounds described herein contain olefinic bonds or other centers of geometric asymmetry, and unless specified otherwise, it is intended that the compounds include both E and Z geometric isomers.

In some embodiments, Ring A is 10-membered heteroaryl. In some embodiments, Ring A is 9-membered heteroaryl. In some embodiments, Ring A is 8-membered heteroaryl.

In some embodiments, Ring A is

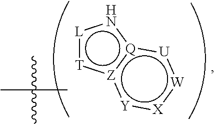

wherein:
L and T is independently C*, N*, CR⁵, N, O, or S;
Z and Q is independently N and C; and
U, W, X, and Y is independently C*, N*, CR⁶ and N; wherein
C* and N* denote attachment points to Ring C.

In some embodiments, Ring A is selected from the group consisting of

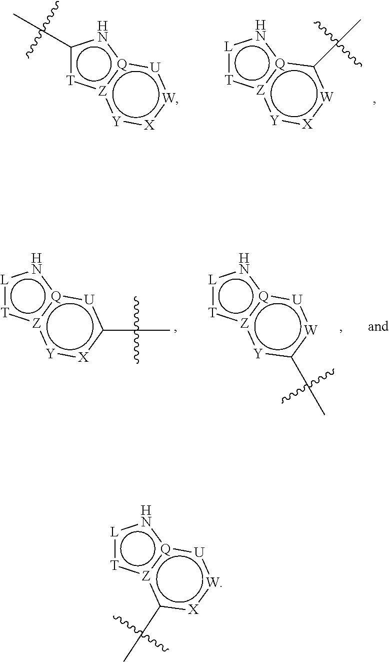

In some embodiments Ring A is

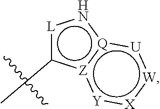

wherein:
L is CR⁶, N, O, or S;
Z and Q is independently N and C; and
U, W, X, and Y is independently CR⁶ and N.

In some embodiments, Ring A is optionally substituted with 1-4 X^{a}, wherein X^{a} is independently selected from the group consisting of halo, C₁₋₆ alkyl, C₁₋₆ haloalkyl, cyano, C₁₋₆ alkoxy, C₁₋₆ haloalkoxy, and SO₂(C₁₋₆ alkyl).

In some embodiments, Ring A is selected from the group consisting of

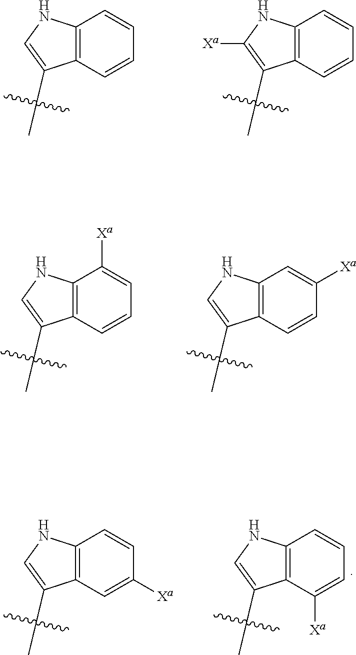

In some embodiments, Ring A is selected from the group consisting of

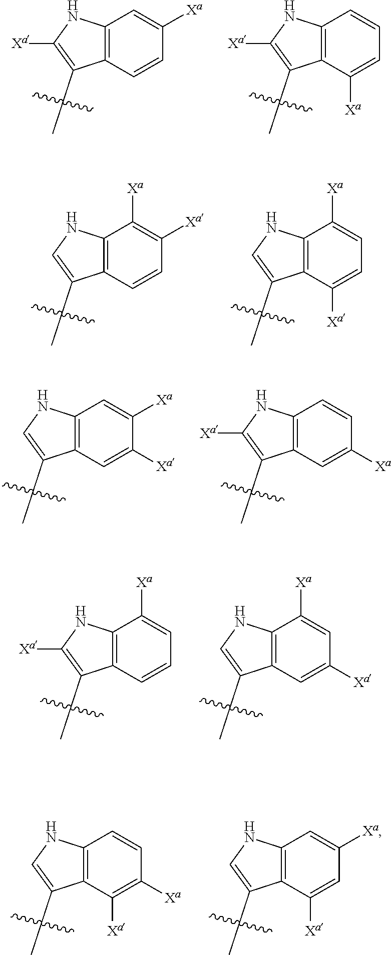

wherein X^{a′} is selected from X^{a}.

In some embodiments, Ring A is selected from the groups consisting of

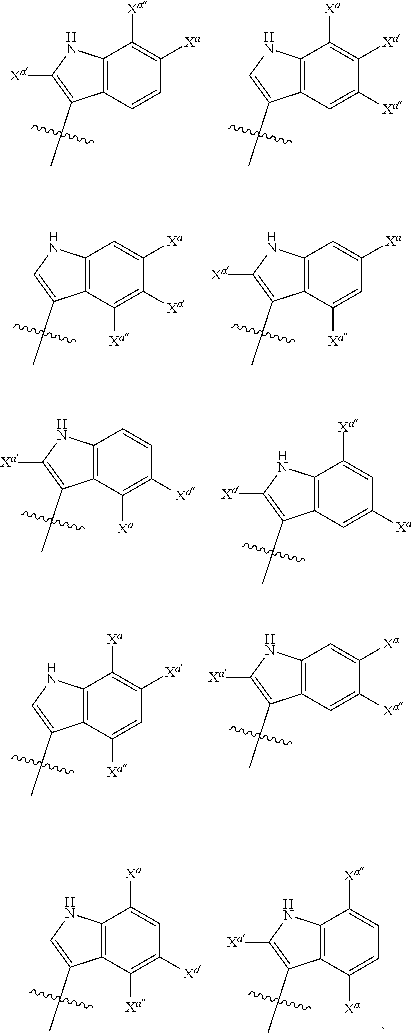

wherein X^{a′} and X^{a″} are independently selected from X^{a}.

In some embodiments, one X^{a} is F, Br, or Cl. In some embodiments, one X^{a} is F. In some embodiments, one X^{a} is Br. In some embodiments, one X^{a} is Cl.

In some embodiments, one X^{a} is C₁₋₆ alkyl. In some embodiments, one X^{a} is C₁₋₃ alkyl.

In some embodiments, one X^{a} is C₁₋₆ haloalkyl. In some embodiments, one X^{a} is C₁₋₃ haloalkyl.

In some embodiments, one X^{a} is C₂₋₆ alkenyl. In some embodiments, one X^{a} is C₂₋₃ alkenyl.

In some embodiments, one X^{a} is C₂₋₆ alkynyl. In some embodiments, one X^{a} is C₂₋₃ alkynyl.

In some embodiments, one X^{a} is —NO₂. In some embodiments, one X^{a} is cyano.

In some embodiments, one X^{a} is C₁₋₆ alkoxy. In some embodiments, one X^{a} is C₁₋₃ alkoxy.

In some embodiments, one X^{a} is C₁₋₆ haloalkoxy. In some embodiments, one X^{a} is C₁₋₃ haloalkoxy. In some embodiments, one X^{a} is C₁₋₆ fluoroalkoxy. In some embodiments, one X^{a} is C₁₋₃ fluoroalkoxy.

In some embodiments, one X^{a} is SO₂(C₁₋₆ alkyl). In some embodiments, one X^{a} is SO₂(C₁₋₃ alkyl).

In some embodiments, one X^{a} is heterocyclyl. In some embodiments, one X^{a} is heteroaryl.

In some embodiments, one X^{a} is C₃₋₆ cycloalkyl.

In some embodiments, one X^{a} is NR¹R². In some embodiments, one X^{a} is C(O)NR¹R².

In some embodiments, one X^{a} is C(O)R³.

In some embodiments, Ring A is selected from the group consisting of

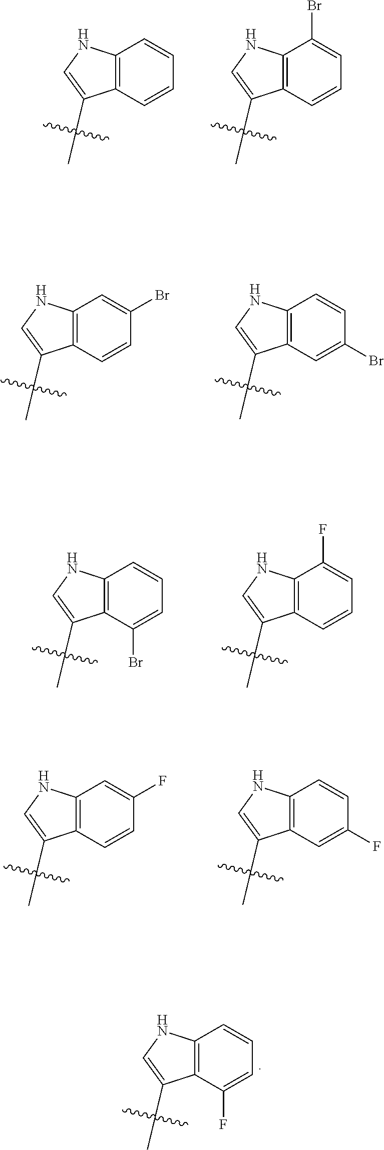

In some embodiments, Ring B is 10-membered heteroaryl. In some embodiments, Ring B is 9-membered heteroaryl. In some embodiments, Ring B is 8-membered heteroaryl.

In some embodiments, Ring B is

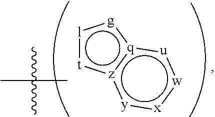

wherein:
l, t and g is C*, N*, CR⁵, NR⁵, N, O, C(═O), or S;
z and q is independently N and C; and
u, w, x, and y is independently C*, N*, CR⁶ and N; wherein
C* and N* denote attachment points to Ring C.

In some embodiments, Ring B is selected from the group consisting of

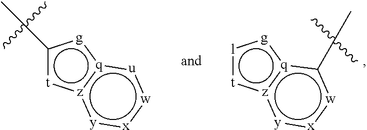

wherein
l, t and g is CR⁵, NR⁶, N, O, C(═O), or S;
z and q is independently N and C; and
u, w, x, and y is independently CR⁶ and N.

In some embodiments, Ring B is selected from the group consisting of

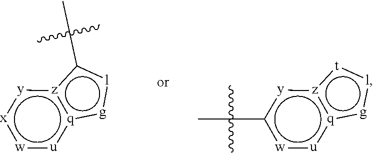

wherein:
l, t and g is CR⁵, NR⁶, N, O, C(═O), or S;
z and q is independently N and C; and
u, w, x, and y is independently CR⁶ and N.

In some embodiments, Ring B is optionally substituted with 1-4 X^{b}, wherein X^{b} is independently selected from the group consisting of halo, C₁₋₆ alkyl, C₁₋₆ haloalkyl, C₂₋₆ alkenyl, C₂₋₆ alkynyl, —NO₂, cyano, C₁₋₆ alkoxy, C₁₋₆ haloalkoxy, SO₂(C₁₋₆ alkyl), heterocyclyl, heteroaryl, C₃₋₆ cycloalkyl, NR¹R², C(O)R³, and C(O)NR¹R².

In some embodiments, Ring B is selected from the group consisting of

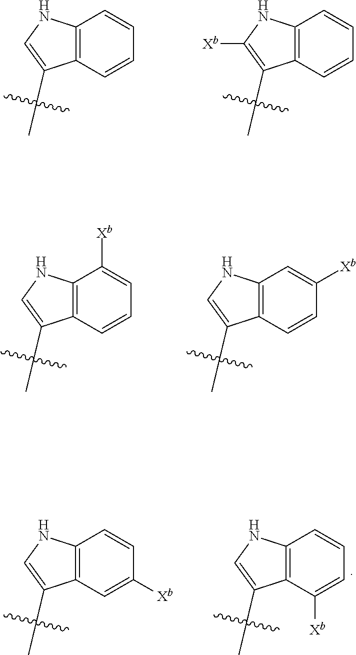

In some embodiments, Ring B is selected from the group consisting of

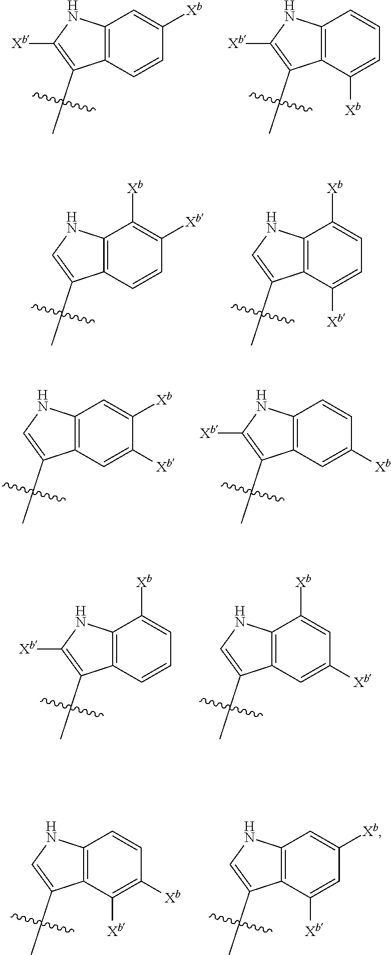

wherein X^{b′} is selected from X^{b}.

In some embodiments, Ring B is selected from the groups consisting of

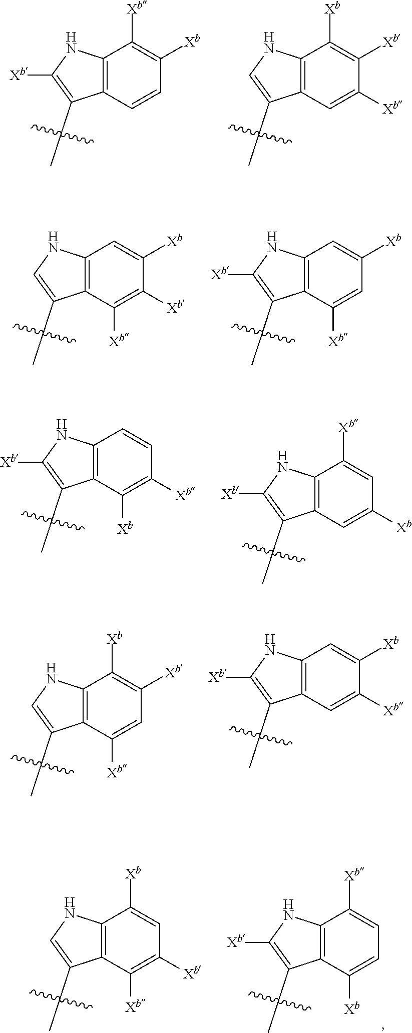

wherein X^{b′} and X^{b″} are independently selected from X^{b}.

In some embodiments, one X^{b} is F, Br, or Cl. In some embodiments, one X^{b} is F. In some embodiments, one X^{b} is Br. In some embodiments, one X^{b} is Cl.

In some embodiments, one X^{b} is C₁₋₆ alkyl. In some embodiments, one X^{b} is C₁₋₃ alkyl.

In some embodiments, one X^{b} is C₁₋₆ haloalkyl. In some embodiments, one X^{b} is C₁₋₃ haloalkyl.

In some embodiments, one X^{b} is C₂₋₆ alkenyl. In some embodiments, one X^{b} is C₂₋₃ alkenyl.

In some embodiments, one X^{b} is C₂₋₆ alkynyl. In some embodiments, one X^{b} is C₂₋₃ alkynyl.

In some embodiments, one X^{b} is —NO₂. In some embodiments, one X^{b} is cyano

In some embodiments, one X^{b} is C₁₋₆ alkoxy. In some embodiments, one X^{b} is C₁₋₃ alkoxy.

In some embodiments, one X^{b} is C₁₋₆ haloalkoxy. In some embodiments, one X^{b} is C₁₋₃ haloalkoxy. In some embodiments, one X^{b} is C₁₋₆ fluoroalkoxy. In some embodiments, one X^{b} is C₁₋₃ fluoroalkoxy.

In some embodiments, one X^{b} is SO₂(C₁₋₆ alkyl). In some embodiments, one X^{b} is SO₂(C₁₋₃ alkyl).

In some embodiments, one X^{b} is heterocyclyl. In some embodiments, one X^{a} is heteroaryl.

In some embodiments, one X^{b} is C₃₋₆ cycloalkyl.

In some embodiments, one X^{b} is NR¹R². In some embodiments, one X^{a} is C(O)NR¹R².

In some embodiments, one X^{b} is C(O)R³.

In some embodiments, Ring B is selected from the group consisting of

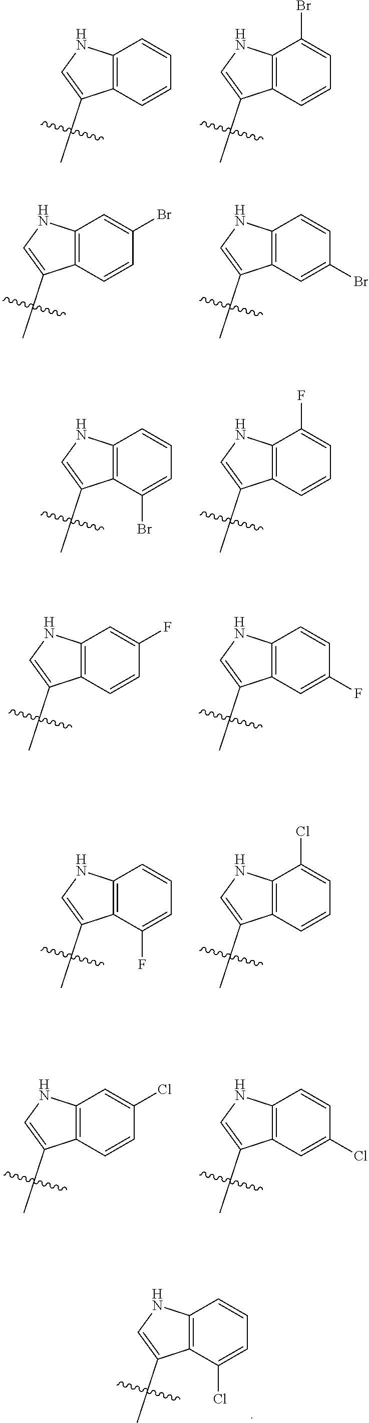

In some embodiments, Ring B is selected from the group consisting of

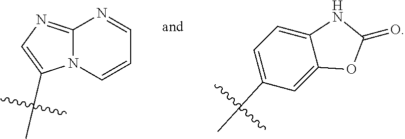 [SMILES: *c1cnc2ncccn12 ; *c1ccc2[nH]c(=O)oc2c1]

In some embodiments, Ring C is 5-membered heteroaryl. In some embodiments, Ring C is 5-membered heteroaryl selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

In some embodiments, Ring C is 5-membered heteroaryl selected from the group consisting of pyrazolyl, oxazolyl, isoxazolyl, isothiazolyl, thiazolyl, oxadiazolyl, and thiadiazolyl.

In some embodiments, Ring C is 5-membered heteroaryl selected from the group consisting of

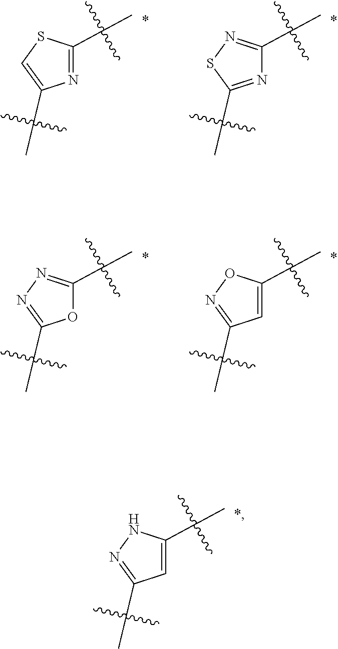 [SMILES: *c1csc(*)n1 ; *c1nsc(*)n1 ; *c1nnc(*)o1 ; *c1cc(*)on1 ; *c1cc(*)[nH]n1]

wherein the asterisk indicates the point of attachment to ring A.

In some embodiments, Ring C is 5-membered heteroaryl selected from the group consisting of

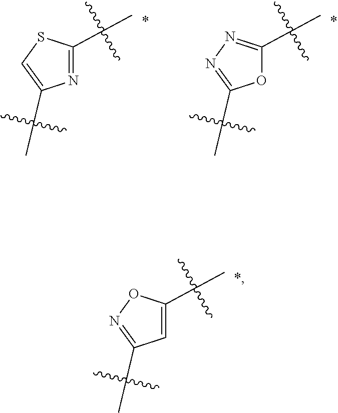 [SMILES: *c1csc(*)n1 ; *c1nnc(*)o1 ; *c1cc(*)on1]

wherein the asterisk indicates the point of attachment to ring A.

In some embodiments, Ring C is

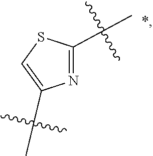 [SMILES: *c1csc(*)n1]

wherein the asterisk indicates the point of attachment to ring A.

In some embodiments, Ring C is

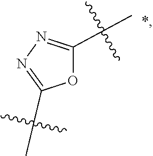 [SMILES: *c1nnc(*)o1]

wherein the asterisk indicates the point of attachment to ring A.

In some embodiments, Ring C is

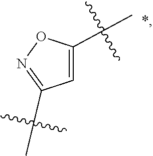 [SMILES: *c1cc(*)on1]

wherein the asterisk indicates the point of attachment to ring A.

In some embodiments, Ring C is

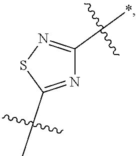

wherein the asterisk indicates the point of attachment to ring A.

In some embodiments, Ring C is 6-membered heteroaryl selected from the group consisting of pyridinyl, pyrimidinyl, pyrazinyl, pyridazinyl, and triazinyl.

In some embodiments, Ring C is 6-membered heteroaryl selected from the group consisting of

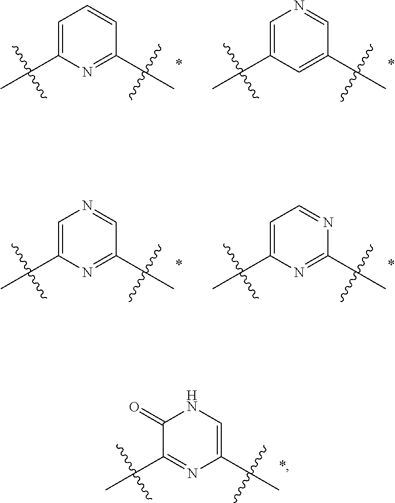 [SMILES: *c1cccc(*)n1 ; *c1cncc(*)c1 ; *c1cncc(*)n1 ; *c1ccnc(*)n1 ; *c1c[nH]c(=O)c(*)n1]

wherein the asterisk indicates the point of attachment to ring A.

In some embodiments, each occurrence of R^{b} is independently selected from the group consisting of: H, C₁₋₄ alkyl, C₁₋₄ haloalkyl, halo, and cyano.

In some embodiments, each occurrence of R¹ and R² is independently selected from the group consisting of: H, C₁₋₄ alkyl, optionally substituted C₃₋₆ cycloalkyl, optionally substituted phenyl, optionally substituted benzyl, C(═O)R⁵, C(═O)OR⁵, and C(═O)NR⁵R⁶; or

In some embodiments, a pair of R¹ and R² on the same nitrogen atom, taken together with said nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of: N, N(R^{a}), O, and S.

In some embodiments, each occurrence of R³ is independently selected from the group consisting of: H, C₁₋₄ alkyl, C₁₋₄ haloalkyl, optionally substituted C₃₋₆ cycloalkyl, and optionally substituted phenyl.

In some embodiments, each occurrence of R⁴ is independently selected from the group consisting of: C₁₋₄ alkyl, C₁₋₄haloalkyl, optionally substituted C₃₋₆ cycloalkyl, optionally substituted phenyl; and optionally substituted benzyl.

In some embodiments, each occurrence of R⁵ and R⁶ is independently selected from the group consisting of H and C₁₋₄ alkyl.

In some embodiments, the compound of Formula (I) is Formula (II)

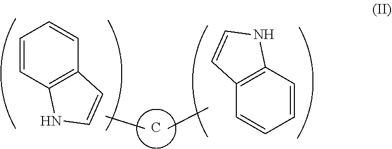

or a pharmaceutically acceptable salt thereof.

In some embodiments the compound of Formula (I) is Formula (II′)

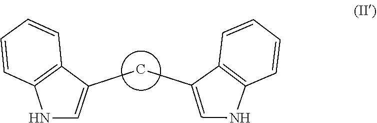 [SMILES: c1ccc2c(Cc3c[nH]c4ccccc34)c[nH]c2c1]

or a pharmaceutically acceptable salt thereof.

In some embodiments of Formula (II′), Ring C is 5-membered heteroaryl.

In some embodiments Formula (II′), Ring C is 5-membered heteroaryl selected from the group consisting of pyrazolyl, oxazolyl, isoxazolyl, isothiazolyl, thiazolyl, oxadiazolyl, and thiadiazolyl.

In some embodiments Formula (II′), Ring C is 6-membered heteroaryl selected from the group consisting of pyridinyl, pyrimidinyl, pyrazinyl, pyridazinyl, and triazinyl.

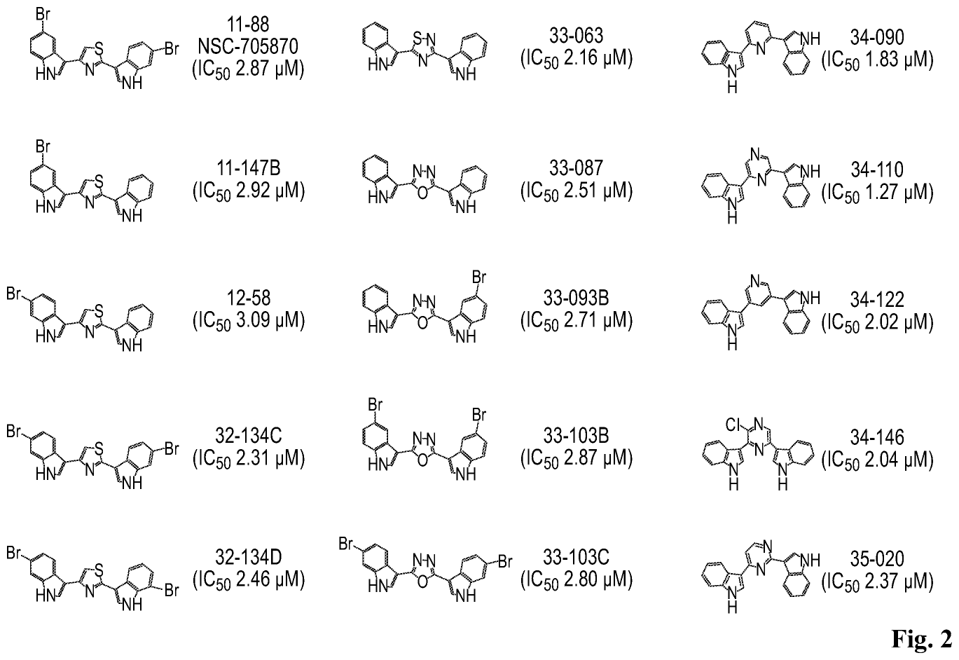

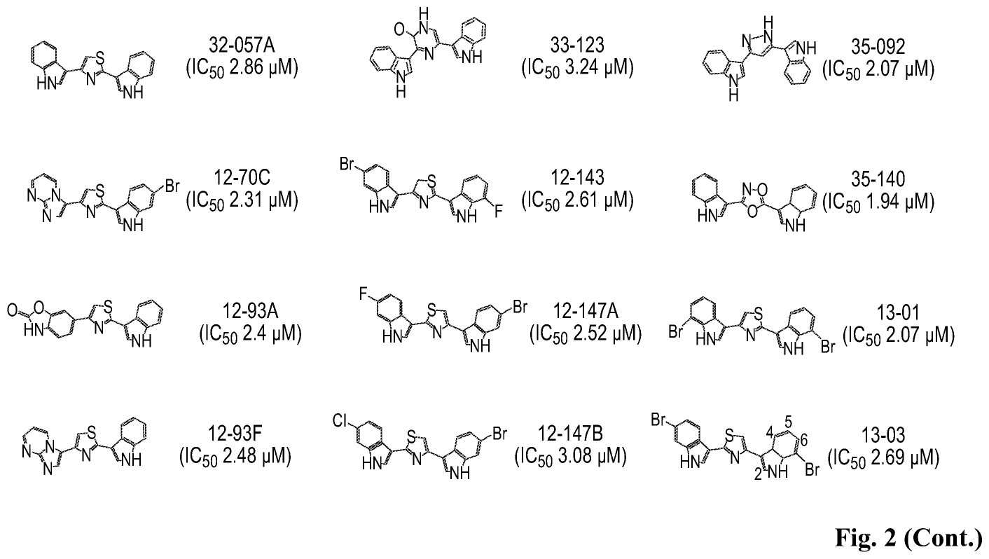

In some embodiments, the compound of Formula (I) is selected from those in FIG. 2 and in the table below.

| Com- | |
|---|---|
| pound | |
| ID | Structure |
| 11-88 | 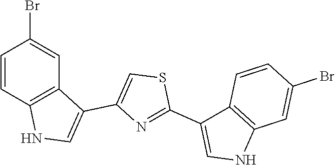 [SMILES: Brc1ccc2c(-c3nc(-c4c[nH]c5ccc(Br)cc45)cs3)c[nH]c2c1] |
| 32-134C | 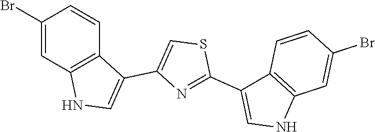 [SMILES: Brc1ccc2c(-c3csc(-c4c[nH]c5cc(Br)ccc45)n3)c[nH]c2c1] |
| 32-134D | 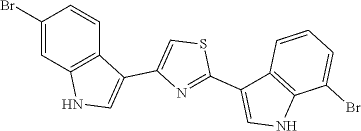 [SMILES: Brc1ccc2c(-c3csc(-c4c[nH]c5c(Br)cccc45)n3)c[nH]c2c1] |
| 12-143 | 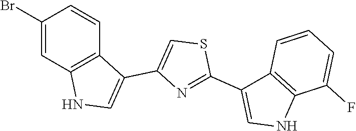 [SMILES: Fc1cccc2c(-c3nc(-c4c[nH]c5cc(Br)ccc45)cs3)c[nH]c12] |
| 33-063 | 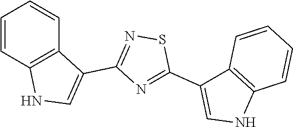 [SMILES: c1ccc2c(-c3nsc(-c4c[nH]c5ccccc45)n3)c[nH]c2c1] |
| 33-093B | 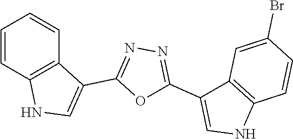 [SMILES: Brc1ccc2[nH]cc(-c3nnc(-c4c[nH]c5ccccc45)o3)c2c1] |
| 11-147B | 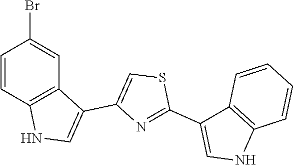 [SMILES: Brc1ccc2[nH]cc(-c3csc(-c4c[nH]c5ccccc45)n3)c2c1] |
| 12-58 | 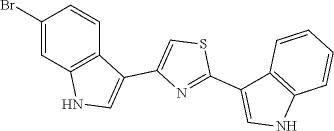 [SMILES: Brc1ccc2c(-c3csc(-c4c[nH]c5ccccc45)n3)c[nH]c2c1] |
| 33-057A | 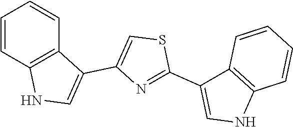 [SMILES: c1ccc2c(-c3csc(-c4c[nH]c5ccccc45)n3)c[nH]c2c1] |
| 12-70C | 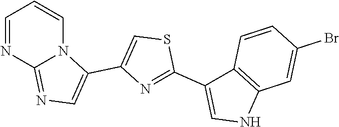 [SMILES: Brc1ccc2c(-c3nc(-c4cnc5ncccn45)cs3)c[nH]c2c1] |
| 12-93A | 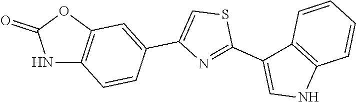 [SMILES: O=c1[nH]c2ccc(-c3csc(-c4c[nH]c5ccccc45)n3)cc2o1] |
| 12-93F | 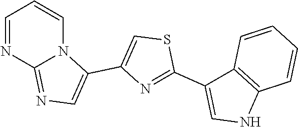 [SMILES: c1ccc2c(-c3nc(-c4cnc5ncccn45)cs3)c[nH]c2c1] |
| 33-087 | 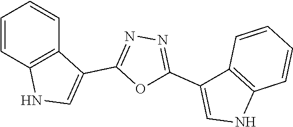 [SMILES: c1ccc2c(-c3nnc(-c4c[nH]c5ccccc45)o3)c[nH]c2c1] |
| 33-103B | 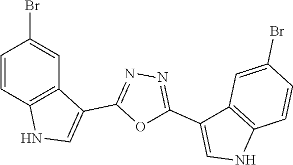 [SMILES: Brc1ccc2[nH]cc(-c3nnc(-c4c[nH]c5ccc(Br)cc45)o3)c2c1] |
| 33-103C | 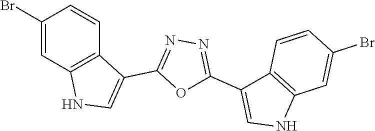 [SMILES: Brc1ccc2c(-c3nnc(-c4c[nH]c5cc(Br)ccc45)o3)c[nH]c2c1] |
| 33-123 | 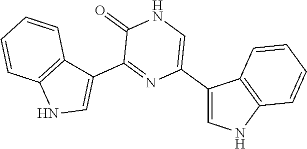 [SMILES: O=c1[nH]cc(-c2c[nH]c3ccccc23)nc1-c1c[nH]c2ccccc12] |
| 12-147A | 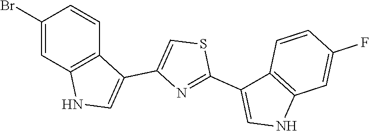 [SMILES: Fc1ccc2c(-c3nc(-c4c[nH]c5cc(Br)ccc45)cs3)c[nH]c2c1] |
| 12-147B | 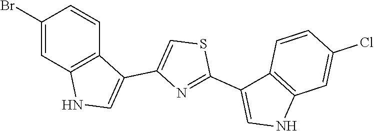 [SMILES: Clc1ccc2c(-c3nc(-c4c[nH]c5cc(Br)ccc45)cs3)c[nH]c2c1] |
| 34-090 | 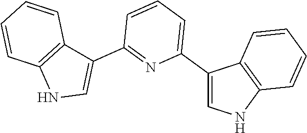 [SMILES: c1cc(-c2c[nH]c3ccccc23)nc(-c2c[nH]c3ccccc23)c1] |
| 34-110 | 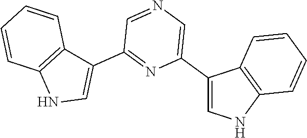 [SMILES: c1ccc2c(-c3cncc(-c4c[nH]c5ccccc45)n3)c[nH]c2c1] |
| 34-122 | 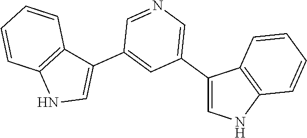 [SMILES: c1ccc2c(-c3cncc(-c4c[nH]c5ccccc45)c3)c[nH]c2c1] |
| 34-146 | 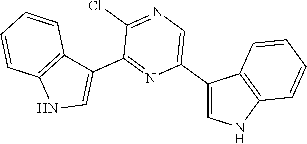 [SMILES: Clc1ncc(-c2c[nH]c3ccccc23)nc1-c1c[nH]c2ccccc12] |
| 35-020 | 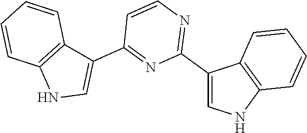 [SMILES: c1ccc2c(-c3ccnc(-c4c[nH]c5ccccc45)n3)c[nH]c2c1] |
| 35-092 | 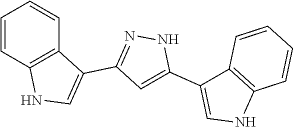 [SMILES: c1ccc2c(-c3cc(-c4c[nH]c5ccccc45)[nH]n3)c[nH]c2c1] |
| 35-140 | 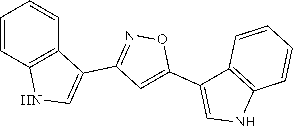 [SMILES: c1ccc2c(-c3cc(-c4c[nH]c5ccccc45)on3)c[nH]c2c1] |
| 13-01 | 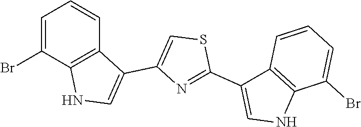 [SMILES: Brc1cccc2c(-c3csc(-c4c[nH]c5c(Br)cccc45)n3)c[nH]c12] |
| 13-03 | 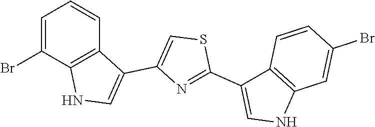 [SMILES: Brc1ccc2c(-c3nc(-c4c[nH]c5c(Br)cccc45)cs3)c[nH]c2c1] |

### Pharmaceutical Compositions and Administration

The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts of the compounds of the disclosure include, for example, acid addition salts which may, for example, be formed by mixing a solution of the compound according to the disclosure with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present disclosure.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present disclosure should be pharmaceutically acceptable salts. Other salts may, however, be useful in the preparation of the compounds according to the disclosure or of their pharmaceutically acceptable salts.

In addition, embodiments of the disclosure include hydrates of the compounds of the present disclosure. The term “hydrate” includes but is not limited to hemihydrate, monohydrate, dihydrate, trihydrate and the like. Hydrates of the compounds of the present disclosure may be prepared by contacting the compounds with water under suitable conditions to produce the hydrate of choice.

In one aspect, the present disclosure provides pharmaceutical compositions comprising the compounds of Formula (I), or their salts, solvates, or stereoisomers thereof, and a pharmaceutically acceptable carrier.

Embodiments of the disclosure also include a process for preparing pharmaceutical products comprising the compounds. The term “pharmaceutical product” means a composition suitable for pharmaceutical use (pharmaceutical composition), as defined herein. Pharmaceutical compositions formulated for particular applications comprising the compounds of the present disclosure are also part of this disclosure, and are to be considered an embodiment thereof.

As such, in another aspect, the present disclosure provides a pharmaceutical composition comprising the compounds of Formula (I) and a pharmaceutically acceptable carrier. In some embodiments, the pharmaceutical composition can further comprise at least one additional therapeutic agent.

In another aspect, the present disclosure provides a pharmaceutical composition comprising a compound of Formula (I) and a pharmaceutically acceptable carrier, in an effective amount, for use as a medicament, e.g., for use in inhibiting (HIF)-1α and/or HIF-2α, or both, in a mammalian cell or population of cells, or for treatment of a condition or disease that is associated with hypoxia, or with increased expression of hypoxia-inducible factor (HIF)-1α and/or HIF-2α, or with both. With respect to pharmaceutical compositions described herein, the pharmaceutically acceptable carrier can be any of those conventionally used, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. Examples of the pharmaceutically acceptable carriers include soluble carriers such as known buffers which can be physiologically acceptable (e.g., phosphate buffer) as well as solid compositions such as solid-state carriers or latex beads. It is preferred that the pharmaceutically acceptable carrier be one which is chemically inert to the active agent(s), and one which has little or no detrimental side effects or toxicity under the conditions of use.

The compositions or pharmaceutical compositions can be administered by any route, including parenteral administration, for example, intravenous, intramuscular, intraperitoneal, or intra-articular injection or infusion, or by sublingual, oral, topical, intranasal, ophthalmic, or transmucosal administration, or by pulmonary inhalation.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, in an embodiment, the compounds of the present disclosure may further comprise, for example, binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular compound, as well as by the particular method used to administer the compound. Accordingly, there are a variety of suitable formulations of the pharmaceutical composition of the disclosure. The following formulations for parenteral, subcutaneous, intravenous, intramuscular, intraarterial, intrathecal and interperitoneal administration are exemplary, and are in no way limiting. More than one route can be used to administer the compounds, and in certain instances, a particular route can provide a more immediate and more effective response than another route.

Suitable soaps for use in parenteral formulations include, for example, fatty alkali metal, ammonium, and triethanolamine salts, and suitable detergents include, for example, (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the compounds in solution. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the disclosure. The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

In some embodiments, the chemical entities described herein or a pharmaceutical composition thereof are suitable for local and topical administration to the eye (e.g., eye drops). Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, the chemical entities described herein or a pharmaceutical composition thereof are suitable for local and topical administration to skin (e.g., ointments and creams). Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

For purposes of the disclosure, the amount or dose of the compounds, salts, solvates, or stereoisomers of any one the compounds of Formula (I), as set forth above, administered should be sufficient to effect, e.g., a therapeutic or prophylactic response, in the subject over a reasonable time frame. The dose will be determined by the efficacy of the particular compound and the condition of a human, as well as the body weight of a human to be treated.

For oral administration, the pharmaceutical compositions can take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations can also contain buffer salts, flavoring, coloring, and sweetening agents as appropriate.

### Methods of Treatment

It is understood by those of ordinary skill, that the compounds of the present disclosure are inhibitors of (HIF)-1 and/or HIF-2, or both, through one or more mechanisms of action. Without being limited to any particular theory, the compounds of the present disclosure can inhibit (HIF)-1 and/or HIF-2, or both, and are therefore useful for treating a condition or disease associated with hypoxia, or with increased expression of hypoxia-inducible factor (HIF)-1α and/or HIF-2α, or with both, the method comprising administering to a subject in need of such treatment a compound of Formula (I) that is capable of inhibiting transcriptional activation mediated by HIFs.

Embodiments of a method for using the disclosed compounds of Formula (I) comprise administering to a subject having, or suspected of having, a condition mediated by hypoxia the compounds of Formula (I) (including pharmaceutically acceptable salts thereof) and/or pharmaceutical compositions thereof. In particular, embodiments of the disclosed compounds of Formula (I) and/or pharmaceutical compositions may be administered to treat or ameliorate conditions in which hypoxia is mediated by (HIF)-1α and/or HIF-2α, or both by inhibiting transcriptional activation mediated by HIFs.

In another aspect, the present disclosure provides the use of the compounds or the pharmaceutical compositions disclosed herein in an amount effective for treating a condition, disease or disorder associated with hypoxia, or with increased expression of hypoxia-inducible factor (HIF)-1α and/or HIF-2α, or with both, the method comprising administering to a subject in need of such treatment a compound of Formula (I) that is capable of inhibiting transcriptional activation mediated by HIFs.

Accordingly, in one aspect, the present disclosure provides a method for treating a condition, disease or disorder associated with hypoxia, or with increased expression of hypoxia-inducible factor (HIF)-1α and/or HIF-2α, comprising administering to the subject a compound of Formula (I), a pharmaceutically acceptable salt thereof, or a pharmaceutical composition thereof. In some embodiments, the method further comprises administering to the subject at least one additional therapeutic agent.

In some embodiments, the present disclosure provides the use of the compounds or the pharmaceutical compositions disclosed herein in an amount effective for treating cancer or a hyperproliferative disease in a subject comprising administering to the subject a pharmaceutical composition comprising a compound of Formula (I). In some embodiments, the use comprises administering to the subject at least one additional therapeutic agent.

Accordingly, in some embodiments, the disclosure provides a method for treating cancer or a hyperproliferative disease in a subject comprising administering to the subject a compound of Formula (I), a pharmaceutically acceptable salt thereof, or a pharmaceutical composition thereof. In certain of these embodiments, the method further comprises administering to the subject at least one additional therapeutic agent.

In some embodiments, the present disclosure provides methods of treating a hyperproliferative disease, such as cancer, which include administering to a subject in need of such treatment an inhibitor of HIF-1 and/or HIF-2 and a chemotherapeutic immunomodulatory agent, such as an immune checkpoint inhibitor.

In another aspect, the present disclosure provides a method for modulating angiogenesis in a subject, comprising administering to the subject a compound of Formula (I), a pharmaceutically acceptable salt thereof, or a pharmaceutical composition thereof. In certain of these embodiments, the method further comprises administering to the subject at least one additional therapeutic agent.

Some embodiments of the disclosed compounds of Formula (I) and pharmaceutical compositions thereof are capable of reducing inflammation by inhibiting HIF-1 activation. In particular, certain embodiments of the disclosed compounds and pharmaceutical compositions thereof have an anti-inflammatory effect. A compound of Formula (I) or a pharmaceutical composition thereof may be formulated for use in human and/or veterinary medicine, and a therapeutically effective amount of the compound of Formula (I) or pharmaceutical composition thereof may be administered to a subject having, or suspected of having, a condition that produces an inflammatory response mediated by hypoxia. Exemplary conditions that produce an inflammatory response mediated by hypoxia include without limitation malignant tumors, intestinal inflammation (e.g., inflammatory bowel disease), lung inflammation (e.g., resulting from acute lung injury), ischemia, atherosclerosis, myocardial infarction, rheumatoid arthritis, and wound healing.

In some embodiments, subjects are selected using specific criteria, such as a definitive diagnosis of an inflammatory condition mediated by hypoxia based on, for example, clinical signs and symptoms and/or laboratory evidence of inflammation. An example of such a subject would be a person having an elevated C-reactive protein level.

In some embodiments, the present disclosure provides the use of the compounds or the pharmaceutical compositions disclosed herein in an amount effective for treating ocular diseases including but not limited to blinding eye disease, e.g., wet (neovascular) AMD, ischemic retinopathies such as diabetic retinopathy, retinal vein occlusions, sickle cell retinopathy, and retinopathy of prematurity, conreal neovascularization, and neovascular glaucoma, in a subject (e.g., a human).

In one aspect, this disclosure features methods of treating an ocular disease, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating a blinding eye disease, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating wet (neovascular) AMD, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating ischemic retinopathies (IRs), which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In certain embodiments, the IR is diabetic retinopathy, retinal vein occlusions, sickle cell retinopathy, and retinopathy of prematurity). In some embodiments, the administering is effected orally or intra-vascularly, or intraocularly, or periocularly, or to the ocular surface.

In one aspect, this disclosure features methods of treating corneal neovascularization, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

In one aspect, this disclosure features methods of treating neovascular glaucoma, which include administering to a subject in need of such treatment, an effective amount of an inhibitor of HIF-1 and/or HIF-2 (e.g., a compound of Formula (I)), or a pharmaceutically acceptable salt thereof, or a pharmaceutical composition thereof. In some embodiments, the administering is effected orally or intra-vascularly, or intraocularly, or periocularly, or to the ocular surface.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

The dose of the compounds, salts, solvates, or stereoisomers of any one the compounds of Formula (I), as set forth above, of the present disclosure also will be determined by the existence, nature and extent of any adverse side effects that might accompany the administration of a particular compound. Typically, an attending physician will decide the dosage of the compound with which to treat each individual patient, taking into consideration a variety of factors, such as age, body weight, general health, diet, sex, compound to be administered, route of administration, and the severity of the condition being treated. By way of example, and not intending to limit the disclosure, the dose of the compound can be about 0.001 to about 1000 mg/kg body weight of the subject being treated/day, from about 0.01 to about 100 mg/kg body weight/day, or from about 1 mg to about 100 mg/kg body weight/day. In some embodiments the dosage of the compound can be in the range of about 0.1 μM to about 100 μM, preferably about 1 μM to about 50 μM.

Alternatively, the compounds of the present disclosure can be modified into a depot form, such that the manner in which the compound is released into the body to which it is administered is controlled with respect to time and location within the body (see, for example, U.S. Pat. No. 4,450,150). Depot forms of compounds can be, for example, an implantable composition comprising the compound and a porous or non-porous material, such as a polymer, wherein the compound is encapsulated by or diffused throughout the material and/or degradation of the non-porous material. The depot is then implanted into the desired location within the body and the compounds are released from the implant at a predetermined rate.

In some embodiments, the compounds of the present disclosure provided herein can be controlled release compositions, i.e., compositions in which the one or more compounds are released over a period of time after administration. Controlled or sustained release compositions include formulation in lipophilic depots (e.g., fatty acids, waxes, oils). In another embodiment the composition is an immediate release composition, i.e., a composition in which all, or substantially all of the compound, is released immediately after administration.

In some embodiments, the compounds of the present disclosure can be delivered in a controlled release system. For example, the agent may be administered using intravenous infusion, an implantable osmotic pump, a transdermal patch, or other modes of administration. In an embodiment, a pump may be used. In some embodiments, polymeric materials can be used. In yet another embodiment, a controlled release system can be placed in proximity to the therapeutic target, i.e., the brain, thus requiring only a fraction of the systemic dose (see, e.g., Design of Controlled Release Drug Delivery Systems, Xiaoling Li and Bhaskara R. Jasti eds. (McGraw-Hill, 2006)).

The compounds included in the pharmaceutical compositions of the present disclosure may also include incorporation of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In accordance with the present disclosure, the compounds of the present disclosure may be modified by, for example, the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection, than do the corresponding unmodified compounds. Such modifications may also increase the compounds' solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently, or in lower doses than with the unmodified compound.

An active agent and a therapeutic agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the disclosure includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

This disclosure contemplates both monotherapy regimens as well as combination therapy regimens. Accordingly, some embodiments of the disclosed pharmaceutical compositions may include a therapeutically effective amount of a second therapeutic agent other than the compound of Formula (I). The second therapeutic may increase the effectiveness of the pharmaceutical composition relative to a pharmaceutical composition comprising only a compound of Formula (I) as an active agent. Exemplary classes of second therapeutic agents include, but are not limited to, anticancer agents, antimalarial agents, antihistamines, antibiotics, antiviral medications, anti-inflammatory agents, and combinations thereof.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

In some embodiments, the second therapeutic agent is an anticancer agent. Suitable anticancer agents include, but are not limited to, chemotherapeutic drug treatment, radiation, gene therapy, hormonal manipulation, immunotherapy and antisense oligonucleotide therapy. Chemotherapeutic agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and bbr3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, hydroxyurea, and mitomycin; topoisomerase inhibitors, such as topotecan and irinotecan; monoclonal antibodies, such as alemtuzumab, bevacizumab, cetuximab, gemtuzumab, rituximab, and tras tuzumab; photosensitizers, such as aminolevulinic acid, methyl aminolevulinate, porfimer sodium, and verteporfin; and other agents, such as alitretinoin, altretamine, amsacrine, anagrelide, arsenic trioxide, asparaginase, bexarotene, bortezomib, celecoxib, denileukin diftitox, erlotinib, estramustine, gefitinib, hydroxycarbamide, imatinib, pentostatin, masoprocol, mitotane, pegaspaigase, and tretinoin. Co-administration of the disclosed compound of Formula (I) with radiation therapy is also contemplated. Methods for treating cancers using radiation therapy are well known in the art.

In other embodiments, the second therapeutic agent is an antimalarial agent. Suitable antimalarial agents include, but are not limited to, quinine, quinidine, cinchoine, cinchonidine, 4-aminoquinolones such as amodiaquine, chloroquine, chloroquine analogs, quinoline analogs, hydroxychloroquine, pyrimethamine, chloroguanide (proguanil), atovaquone (available as Malarone, a combination of atovaquone and proguanil), sulfonamides such as sulfadoxine and sulfamehtoxypyridazine, mefloquine, primaquine, artemisinin, artemisinin derivatives such as artesunate, artemether, arteether, and dihydroartemisinin, halofantrine (a phenanthrene methanol), Halfan, doxycycline, tetracycline, clindamycin, prodiginines, and combinations thereof.

In still other embodiments, the second therapeutic agent is an anti-inflammatory agent. Exemplary anti-inflammatory agents include non-steroidal anti-inflammatory drugs (NSAIDs) such as ibuprofen, ketoprofen, piroxicam, naproxen, sulindac, aspirin, choline subsalicylate, diflunisal, fenoprofen, indomethacin, meclofenamate, salsalate, tolmetin, ketorolac, flurbiprofen, and magnesium salicylate.

The compounds of the present disclosure can optionally be employed in combination with one or more active agents selected from STING agonist compounds, anti-viral compounds, antigens, adjuvants, CTLA-4 and PD-1 pathway antagonists and other immunomodulatory agents, lipids, liposomes, peptides, anti-cancer agents, and chemotherapeutic agents including but not limited to PARP inhibitors, ACAT1 inhibiting compounds, autophagy inhibiting compounds, tyrosine kinase and signaling kinase inhibitors (such as AKT, MEK), cell cycle inhibitors (such as CDK4/6, Wee1, Plk, Aurora kinase), and chromatin modifiers.

Further examples of additional therapeutic agents include, without limitation, enzymes, receptor antagonists or agonists, hormones, growth factors, autogenous bone marrow, antibiotics, antimicrobial agents, RNA and DNA molecules and nucleic acids, and antibodies. Specific examples of useful therapeutic agents the above categories include: anti-neoplastics such as androgen inhibitors, antimetabolites, cytotoxic agents, and immunomodulators.

Further examples of alkylating antineoplastic agents include carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; vinca alkaloid natural antineoplastics, such as vinblastine and vincristine, and PD1 inhibitors such as lambrolizumab.

The compounds of the present disclosure can optionally be employed in combination with one or more HIF-1 and/or HIF-2 inhibitors, including but not limited to benzopyranyl triazole, BIX01294, cardenolides, CRLX-101, EZN-2208, glyceollins, IDF-1174, LBH589, MPTOG157, vorinostat, NNC 55-0396, kresoxim-methyl analogues, belzutifan, and PT2399.

In certain embodiments, the additional chemotherapeutic agent is an immunomodulatory moiety, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155; e.g., CTLA-4 orPD1 or PD-L1). See, e.g., Postow, M. J. Clin. Oncol. 2015, 33, 1.

Immune checkpoint inhibitors include but not limited to Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, Durvalumab, Ipilimumab, REGN2810, BMS-936558, SHR1210, KN035, IBI308, BGB-A317, BCD-100, JS001 and MGA271.

As another example, methods of treating an ocular disease (e.g., a blinding eye disease) as described herein can further include administering an anti-vascular endothelial growth factor (VEGF) agent, an angiotensin-converting enzyme (ACE) inhibitor, a peroxisome proliferator-activated receptor (PPAR)-gamma agonist, a renin inhibitor, a steroid, an agent that modulates autophagy, semapimod, a MIF inhibitor, a CCR2 inhibitor, CKR-2B, a 2-thioimidazole, CAS 445479-97-0, CCX140, clodronate, a clodonate-liposome preparation and gadolinium chloride.

In some embodiments, the present disclosure provides the use of the compounds or the pharmaceutical compositions disclosed herein in an amount effective for treatment or prevention of Von Hippel-Lindau disease, or any other disease where HIF-1, HIF-2 or hypoxia response is identified as a potential mechanism for therapeutic targeting.

Embodiments of a method for using the disclosed compounds of Formula (I) comprise inhibiting HIF-1 and/or HIF-2, or both activity by contacting a cell with an effective amount of a compound of Formula (I) (including pharmaceutically acceptable salts thereof). The cell may be contacted in vitro or in vivo with the compound of Formula (I). The cell also may be contacted with a second therapeutic agent. In some embodiments, contacting the cell comprises administering to a subject a therapeutically effective amount of (i) the compound of Formula (I) or pharmaceutically acceptable salt thereof, or (ii) a pharmaceutical composition comprising the compound of Formula (I) or pharmaceutically acceptable salt thereof and at least one pharmaceutically acceptable carrier.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

As used herein, the term “modulate” means that the compounds of Formula (I), described herein either increase or decrease activity, e.g., of angiogenesis.

As used herein, the term “hyperproliferative disease” includes cancer and other diseases such as neoplasias and hyperplasias. Cellular proliferative diseases include, for example, Rheumatoid Arthritis, Inflammatory Bowel Disease, Osteoarthritis, Leiomyomas, Adenomas, Lipomas, Hemangiomas, Fibromas, Vascular Occlusion, Restenosis, Artherosclerosis, a Pre-neoplastic Lesion, Carcinoma in situ, Oral Hairy Leukoplakia, or Psoriasis.

In accordance with one or more embodiments, the term “cancer” includes but is not limited to, any of the following: Acute lymphoblastic leukemia (ALL), Acute myeloid leukemia, Adrenocortical carcinoma, AIDS-related cancers, AIDS-related lymphoma, Anal cancer, Appendix cancer, Astrocytoma, childhood cerebellar or cerebral, Basal-cell carcinoma, Bile duct cancer, extrahepatic (see cholangiocar-cinoma), Bladder cancer Bone tumor, osteosarcoma/malignant fibrous histiocytoma, Brainstem glioma Brain cancer, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Breast cancer, Bronchial adenomas/carcinoids, Burkitt's lymphoma, Carcinoid tumor, childhood, Carcinoid tumor, gastrointestinal, Carcinoma of unknown primary, Cerebellar astrocytoma, Cerebral astrocytoma/malignant glioma, Cervical cancer, Chondrosarcoma, Chronic lymphocytic leukemia, Chronic myelogenous leukemia, Chronic myeloproliferative disorders, Colon cancer, Cutaneous T-cell lymphoma, Desmoplastic small round cell tumor, Endometrial cancer, Ependymoma, Esophageal cancer, Ewing's sarcoma, Extracranial germ cell tumor, Extragonadal germ cell tumor, Extrahepatic bile duct cancer, intraocular melanoma, retinoblastoma, Gallbladder cancer, Gastric (stomach) cancer, Gastrointestinal carcinoid tumor, Gastrointestinal stromal tumor (GIST), Germ cell tumor: extracranial, extragonadal, or ovarian, Gestational trophoblastic tumor, Glioma of the brain stem, Glioma, childhood cerebral astrocytoma, Glioma, childhood visual pathway and hypothalamic, Gastric carcinoid, Hairy cell leukemia, Hepatocellular (liver) cancer, Hodgkin lymphoma, Hypopharyngeal cancer, Hypothalamic and visual pathway glioma, childhood, Intraocular melanoma, Islet cell carcinoma (endocrine pancreas), Kaposi sarcoma, Kidney cancer (renal cell cancer), Laryngeal cancer, Leukaemias, Leukaemia, acute lymphoblastic (also called acute lymphocytic leukaemia), Leukaemia, acute myeloid (also called acute myelogenous leukemia), Leukaemia, chronic lymphocytic (also called chronic lymphocytic leukemia), Leukemia, chronic myelogenous (also called chronic myeloid leukemia), Leukemia, hairy cell, Lip and oral cavity cancer, Liposarcoma, Liver cancer (primary), Lung cancer, non-small cell, Lung cancer, small cell, Lymphomas, Lymphoma, AIDS-related, Lymphoma, Burkitt, Lymphoma, cutaneous T-Cell, Lymphoma, Hodgkin, Lymphomas, Non-Hodgkin, Lymphoma, primary central nervous system, Macroglobulinemia, Waldenstrom, Malignant fibrous histiocytoma of bone/osteosarcoma, Medulloblastoma, Melanoma, Merkel cell cancer, Mesothelioma, Mouth cancer, Multiple endocrine neoplasia syndrome, Multiple myeloma/plasma cell neoplasm, Mycosis fungoides, Myelodysplastic syndromes Myelodysplastic/myeloproliferative diseases, Myelogenous leukemia, chronic Myeloid leukemia, adult acute, Myeloid leukemia, childhood acute, Myeloma, multiple (cancer of the bone-marrow), Myeloproliferative disorders, chronic, Myxoma, Nasal cavity and paranasal sinus cancer, Nasopharyngeal carcinoma Neuroblastoma, Non-Hodgkin lymphoma, Non-small cell lung cancer, Oligodendroglioma, Oral cancer, Oropharyngeal cancer, Osteosarcoma/malignant fibrous histiocytoma of bone, Ovarian cancer, Ovarian epithelial cancer (surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma Plasma cell neo-plasia/Multiple myeloma, Pleuropulmonary blastoma, Primary central nervous system lymphoma, Prostate cancer, Rectal cancer, Renal cell carcinoma, Renal pelvis and ureter, transitional cell cancer, Rhabdomyosarcoma, childhood, Salivary gland cancer, Sarcoma, Ewing family of tumors, Sarcoma, Kaposi, Sarcoma, soft tissue, Sarcoma, uterine, Sezary syndrome, Skin cancer (non-melanoma), Skin cancer (melanoma), Skin carcinoma, Merkel cell, Small cell lung cancer, Small intestine cancer, Soft tissue sarcoma, Squamous cell carcinoma-see skin cancer (non-melanoma), Squamous neck cancer with occult primary, metastatic, Stomach cancer, Supratentorial primitive neuroectodermal tumor, childhood, T-Cell lymphoma, cutaneous, Testicular cancer, Throat cancer, Thymoma, Thymoma and thymic carcinoma, Thyroid cancer, Thyroid cancer, Transitional cell cancer of the renal pelvis and ureter, Trophoblastic tumor, gestational, Unknown primary site, Ureter and renal pelvis, transitional cell cancer, Urethral cancer, Uterine cancer, endometrial, Uterine sarcoma, Vaginal cancer, Visual pathway and hypothalamic glioma, Vulvar cancer, Waldenstrom macroglobulinemia and/or Wilms tumor (kidney cancer).

As used herein, the term “blinding eye disease” refers to one of various ophthalmic diseases and includes diseases of the eye and the ocular adnexa. Blinding eye disease includes, but is not limited to, age-related macular degeneration (AMD), e.g., non-exudative AMD, neovascular AMD, ischemic retinopathies, including, but not limited to, diabetic retinopathy, retinal vein occlusions, sickle cell retinopathy, and retinopathy of prematurity, and ocular neovascularization in the context of inflammatory eye disease (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), and diffuse subretinal fibrosis (DSF)) or central serous retinopathy (CSR), corneal neovascularization, neovascular glaucoma, and pterygia, as well as intraocular or periocular tumors, including, but not limited to uveal melanoma, conjunctival melanoma, retinoblastoma, and conjunctival squamous cell carcinoma.

In various embodiments, the blinding eye disease is AMD. AMD is the leading cause of blindness in the developed world. Early AMD is characterized by the accumulation of drusen, the hallmark of disease, while geographic atrophy (GA) and choroidal neovascularization (CNV) are the blinding complications of late non-exudative (so-called “dry” or atrophic) and exudative (so-called “wet” or neovascular) disease, respectively. Anti-VEGF therapy has revolutionized treatment of wet AMD, but represents just 12-15% of all AMD cases. Though vitamin supplementation can slow the rates of progression no treatments exist for dry AMD.

In various embodiments, the blinding eye disease is wet AMD. Wet AMD is characterized by the growth of new blood vessels beneath the retina or RPE which can bleed and leak fluid, resulting in a rapid and often severe loss of central vision in the majority cases. This loss of central vision adversely affects one's everyday life by impairing the ability to read, drive and recognize faces. In some cases, the macular degeneration progresses from the dry form to the wet form.

In one embodiment, the blinding eye disease is exudative or wet AMD. In one embodiment, the blinding eye disease is associated with choroidal neovascularization (CNV).

In one embodiment, the blinding eye disease is ischemic retinopathy, including diabetic retinopathy, retinal vein occlusions, sickle cell retinopathy, and retinopathy of prematurity. In one embodiment, the blinding eye disease is associated with retinal neovascularization or macular edema.

In one embodiment, the blinding eye disease is corneal neovascularization. In one embodiment, the blinding eye disease is associated with corneal neovascularization (CoNV).

In one embodiment, the blinding eye disease is neovascular glaucoma. In one embodiment, the blinding eye disease is associated with neovascularization of the iris (NVI) or angle (NVA).

In other embodiments, the blinding eye disease is an idiopathic disorder that may, without wishing to be bound by theory, be characterized by retinal inflammation, with or without accompanying macular degeneration, including, but not limited to, diabetic retinopathy, retinal vein occlusions, sickle cell retinopathy, and retinopathy of prematurity, white-dot syndromes (e.g. serpiginous chorioretinopathy, serpiginous retinopathy, acute posterior multifocal placoid pigment epitheliopathy (APMPPE), multiple evanescent white dot syndrome (MEWDS), acute zonal occult outer retinopathy (AZOOR), punctate inner choroidopathy (PIC), and diffuse subretinal fibrosis (DSF)).

In other embodiments, the blinding eye disease is central serous retinopathy (CSR). CSR is a fluid detachment of macula layers from their supporting tissue. CSR is often characterizable by the leak and accumulation of fluid into the subretinal or sub-RPE space. Without wishing to be bound by theory, the leak and accumulation of fluid may occur because of small breaks in the RPE.

In addition to treating pre-existing blinding eye diseases, the present disclosure comprises prophylactic methods in order to prevent or slow the onset of these disorders. In prophylactic applications, an agent can be administered to a subject susceptible to or otherwise at risk of a particular blinding eye disease. Such susceptibility may be determined by, for example, familial predisposition, genetic testing, risk factor analysis, blood or other cytokine or biomarker levels, and ocular examination, which can include multi-modal analysis such as FAF, blue light, white light, red-free, near infra-red, infrared, DNIRA, etc. Such susceptibility may also be determined by, for example, detection by OCT, with cross-sectional, three-dimensional and en face viewing.

As used herein, the term “treating” or “treatment” refers to reducing the symptoms or arresting or inhibiting further development of the disease (in whole or in part). “Treating” or “treatment” includes any effect, e.g., lessening, reducing, modulating, or eliminating, that results in the improvement of the disease and the like. For example, certain methods herein treat cancer by decreasing or reducing the occurrence, growth, metastasis, or progression of cancer or decreasing a symptom of cancer.

As used herein, the term an “effective amount” is an amount sufficient to accomplish a stated purpose (e.g. achieve the effect for which it is administered, treat a disease, reduce enzyme activity, increase enzyme activity, or reduce one or more symptoms of a disease). An example of an “effective amount” is an amount sufficient to contribute to the treatment, prevention, or reduction of a symptom or symptoms of a disease, which could also be referred to as a “therapeutically effective amount.” A “prophylactically effective amount” of a drug is an amount of a drug that, when administered to a subject, will have the intended prophylactic effect, e.g., preventing or delaying the onset (or reoccurrence) of a disease, or reducing the likelihood of the onset (or reoccurrence) of a disease or its symptoms.

As used herein, the term a “reduction” of a symptom or symptoms means decreasing of the severity or frequency of the symptom(s), or the complete elimination of the symptom(s).

As used herein, the term “Contacting” refers to the process of allowing at least two distinct species to become sufficiently proximal to react, interact, and/or physically touch. It should be appreciated, however, that the resulting reaction product can be produced directly from a reaction between the added reagents or from an intermediate from one or more of the added reagents which can be produced in the reaction mixture. The term “contacting” includes allowing two species to react, interact, and/or physically touch, wherein the two species may be a compound as described herein and a drug target, e.g., HIF-1α and/or HIF-2α.

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction in the progression of a disease and/or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in transcriptional activation mediated by HIFs.

As used herein, the term “Disease” refers to a state of being or health status of a subject or subject capable of being treated with a compound, pharmaceutical composition, or method provided herein. In some embodiments, the compounds and methods described herein comprise reduction or elimination of one or more symptoms of the disease, e.g., through administration of a compound described herein, a pharmaceutically acceptable salt thereof, or a pharmaceutical composition comprising a compound described herein, or a pharmaceutically acceptable salt thereof.

As used herein, the term “HIF-1” refers to transcriptional regulator hypoxia-inducible factor-1 (HIF-1), a heterodimeric complex protein consisting of an oxygen regulated subunit (HIF-1α) and a constitutively expressed nuclear subunit (HIF-13).

As used herein, the term “HIF-2” refers to transcriptional regulator hypoxia-inducible factor-2 (HIF-2), a heterodimeric complex protein consisting of an oxygen regulated subunit (HIF-2α) and a constitutively expressed nuclear subunit (HIF-1β).

### EXAMPLES

Exemplary compounds were prepared via several general synthetic routes set forth in the examples below. Any of the disclosed compounds of the present invention can be prepared according to one or more of these synthetic routes or specific examples, or via modifications thereof accessible to the person of ordinary skill in the art.

All commercially available reagents and solvents were used without further purification unless otherwise stated. Automated flash chromatography was performed on a Teledyne Isco CombiFlash Rf+ or Grace Reveleris using Teledyne Isco or Grace/Buchi flash silica and/or C18 cartridges. Spectra were recorded on a Bruker Avance-III spectrometer (¹H NMR at 500 MHz and ¹³C NMR at 125 MHz) at 296 K in CDCl3 (¹H NMR referenced to internal standard tetramethylsilane 0 ppm, ¹³C NMR referenced to 77.00 ppm), d6-DMSO (¹H NMR referenced to 2.50 ppm, ¹³C NMR referenced to 39.510 ppm). Analytical LC-MS was performed using Agilent 1260 equipped with autosampler (Agilent Poroshell 120 column (50×3.0 mm I.D., 2.7 m); 0.05% TFA in water/acetonitrile gradient; UV detection at 220 and 254 nm) and electro spray ionization. Unless otherwise noted, all final compounds showed purity greater than 95% at 215 and 254 nm using this method.

### Synthesis of 7-bromo-1H-indole-3-carbonitrile

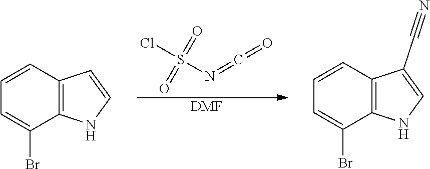

To a rapidly stirred solution of 7-bromoindole (5.0 g, 25.5 mmol, 1.0 equiv.) in anhydrous DMF (17 ml) was added chlorosulfonyl isocyanate (2.4 ml, 28.1 mmol, 1.1 equiv.) dropwise while cooling with an ice water bath. Once addition was complete, the ice water bath was allowed to equilibrate to rt overnight (ca. 18 h). The reaction was added slowly to rapidly stirred ice water resulting in a precipitate. The solid is filtered, washed with water and dried under vacuum to provide 7-bromo-1H-indole-3-carbonitrile as a red-brown solid (5.32 g, 94%). This material was used without further purification. ¹H NMR (500 MHz, DMSO-d6) δ 8.28 (s, 1H), 7.76 (s, 1H), 7.60 (d, J=8.49 Hz, 1H), 7.37 (dd, J=1.41, 8.49 Hz, 1H). ¹³C NMR (126 MHz, DMSO-d6) δ 136.1, 135.6, 125.7, 124.6, 120.2, 115.9, 115.8, 115.6, 84.6. LCMS t_{R}=2.33 min, m/z 220.9, 222.8 [M+H]⁺ 242.9, 244.9 [M+Na]⁺.

### Synthesis of 7-bromo-1H-indole-3-carbothioamide

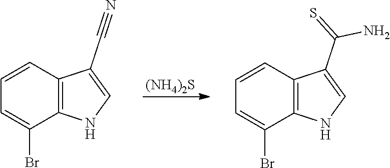

To a 5 ml Biotage vial were charged 7-bromo-1H-indole-3-carbonitrile (398 mg, 1.80 mmol, 1.00 equiv.), 20 wt % aq. (NH₄)₂S (3.1 ml, 9.0 mmol, 5.0 equiv.) and MeOH (0.6 mL). After sealing the vial, the suspension was irradiated in a Biotage Initiator microwave for 3 h at 100° C. After cooling the reaction mixture, the precipitate was filtered, washed with water and dried under vacuum to obtain pale yellow crystals (438 mg, 95% yield) of 7-bromo-1H-indole-3-carbothioamide. This material was used in the next step without further purification. ¹H NMR (500 MHz, DMSO-d6) δ 11.84 (br. s., 1H), 9.03 (br. s., 1H), 8.90 (br. s., 1H), 8.59 (d, J=8.65 Hz, 1H), 8.09 (s, 1H), 7.62 (d, J=1.73 Hz, 1H), 7.27 (dd, J=1.73, 8.65 Hz, 1H). ¹³C NMR (126 MHz, DMSO-d6) δ 193.2, 137.7, 128.4, 125.2, 123.7, 123.6, 116.3, 114.8, 114.6. LCMS t_{R}=2.07 min. m/z 257.0, 254.9 [M+H]⁺.

### Synthesis of 2-bromo-1-(6-bromo-1-tosyl-1H-indol-3-yl)ethan-1-one

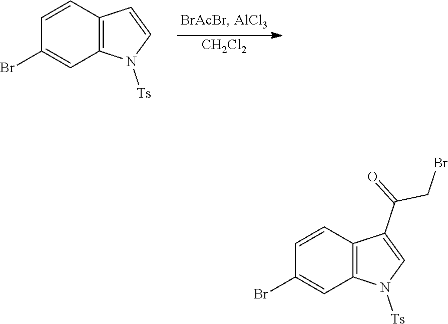

To a suspension of AlCl₃ (30.17 mmol) in CH₂Cl₂ (60 mL) was added bromoacetyl bromide (1440 uL, 16.5 mmol) at rt for 15 min. To this mixture was added a solution of 6-bromo-1-tosyl-1H-indole (7.31 mmol) in CH₂Cl₂ (10 mL) dropwise at 0° C. The mixture was stirred at 0° C. for 1 h and poured onto ice (60 g). The aqueous layer was extracted with CH₂C12. The organic extracts were washed with saturated aq. NaHCO₃, brine and dried over anhydrous Na₂SO₄. After removal of the solvent, the crude product was recrystallized with CHCl₃/EtOH and column chromatographed with methylene chloride/hexane to afford 2-bromo-1-(6-bromo-1-tosyl-1H-indol-3-yl)ethan-1-one (2.32 g, 67% yield) as a white solid. ¹H NMR (500 MHz, CHLOROFORM-d) δ 8.30 (s, 1H), 8.16 (d, J=8.49 Hz, 1H), 8.12 (d, J=1.57 Hz, 1H), 7.82-7.87 (m, J=8.49 Hz, 2H), 7.48 (dd, J=1.65, 8.57 Hz, 1H), 7.30-7.37 (m, J=8.17 Hz, 2H), 4.33 (s, 2H), 2.41 (s, 3H); ¹³C NMR (125.7 MHz, CHLOROFORM-d) δ 186.8, 146.6, 135.4, 134.0, 133.0, 130.6, 128.6, 127.2, 126.4, 124.3, 120.0, 117.8, 116.3, 31.1, 21.8; LCMS m/z 471.6 [M+H]⁺.

### Synthesis of 4-(6-bromo-1-tosyl-1H-indol-3-yl)-2-(7-bromo-1H-indol-3-yl)thiazole

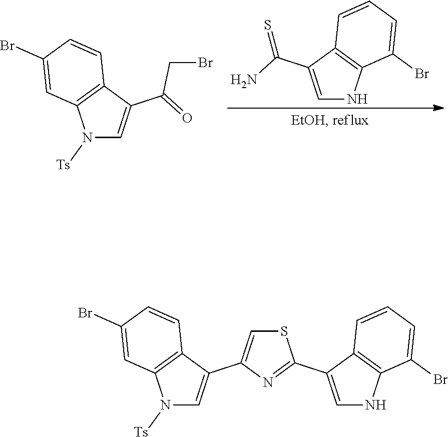 [SMILES: Cc1ccc(S(=O)(=O)n2cc(C(=O)CBr)c3ccc(Br)cc32)cc1 ; NC(=S)c1c[nH]c2c(Br)cccc12 ; Cc1ccc(S(=O)(=O)n2cc(-c3csc(-c4c[nH]c5c(Br)cccc45)n3)c3ccc(Br)cc32)cc1]

A suspension of 7-bromo-1H-indole-3-carbothioamide (815 mg, 3.19 mmol) and 2-bromo-1-(6-bromo-1-tosyl-1H-indol-3-yl)ethan-1-one4 (1.58 g, 3.35 mmol) in EtOH (100 ml) was refluxed for 30 min. The mixture was cooled and the precipitates were filtered, washed with cold EtOH (20 mL×2) and dried under vacuum to provide 4-(6-bromo-1-tosyl-1Hindol-3-yl)-2-(7-bromo-1H-indol-3-yl)thiazole (2.00 g, 99.9% yield) as a white solid. This material was used in the next step without further purification.

### Example 1: 4-(6-bromo-1H-indol-3-yl)-2-(7-bromo-1H-indol-3-yl)thiazole (32-134D

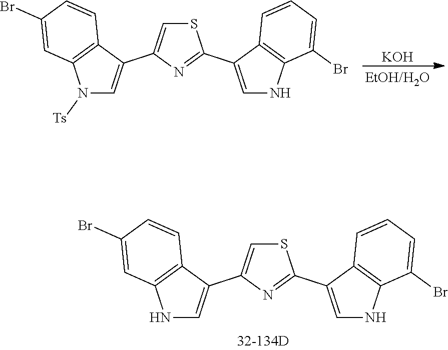

To a suspension of 4-(6-bromo-1-tosyl-1H-indol-3-yl)-2-(7-bromo-1H-indol-3-yl)thiazole (2.00 g, 3.19 mmol) in ethanol (40 ml) and water (40 ml), was added KOH (4 g, 5% W/V). The mixture was heated at reflux under nitrogen until complete (1 day). Water (40 mL) was added at RT to dilute the mixture. The reaction mixture was filtered, washed with water (30 mL×3) and the precipitate was dried. The precipitate was dissolved with EtOAc and EtOH and absorbed unto silica gel. Column chromatography (Hexane/(EtOAc+10% EtOH)) provided 4-(6-bromo-1H-indol-3-yl)-2-(7-bromo-1H-indol-3-yl)thiazole (32-134D) (1.20 g, 79.4% yield in two steps overall). ¹H NMR (500 MHz, DMSO-d6) δ 12.00 (br. s., 1H), 11.54 (br. s., 1H), 8.39 (d, J=8.02 Hz, 1H), 8.18 (d, J=8.65 Hz, 1H), 8.17 (s, 1H), 8.04 (s, 1H), 7.69 (s, 1H), 7.67 (s, 1H), 7.49 (d, J=7.55 Hz, 1H), 7.30 (d, J=8.65 Hz, 1H), 7.21 (t, J=7.78 Hz, 1H); ¹³C-NMR (125.7 MHz, DMSO-d6) δ 161.8, 150.5, 138.0, 135.4, 127.9, 126.5, 126.3, 125.6, 124.2, 123.0, 122.7, 122.4, 120.6, 114.9, 114.8, 112.4, 111.9, 107.7, 105.3; LCMS m/z 471.6 [M+H]⁺.

### Example 2: 3,5-di(1H-indol-3-yl)-1,2,4-thiadiazole (33-063

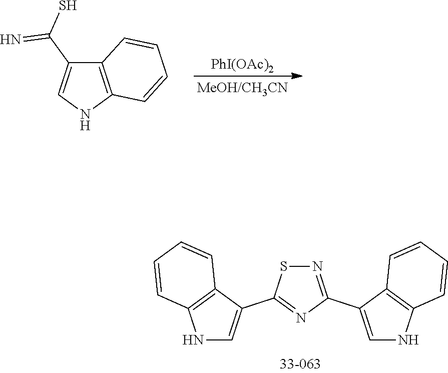 [SMILES: "PhI(OAc)2" ; N=C(S)c1c[nH]c2ccccc12 ; c1ccc2c(-c3nsc(-c4c[nH]c5ccccc45)n3)c[nH]c2c1]

Iodobenzene diacetate (1.02 g, 2.41 mmol) was added to a stirred solution of indole-3-thiocarboxamide (416 mg, 2.36 mmol) in MeOH/CH₃CN (12 mL/12 mL) at 25° C. The reaction was complete within 20 min. The reaction mixture was concentrated and filtered through celite washing with EtOAc. The EtOAc was concentrated in vacuo and the residue was purified by column chromatography on silica gel. 3,5-di(1H-indol-3-yl)-1,2,4-thiadiazole was obtained in a (275 mg, 74% yield). ¹H NMR (500 MHz, DMSO-d6) δ 12.13 (br. s., 1H), 11.72 (br. s., 1H), 8.47 (m, 1H), 8.43 (s, 1H), 8.27 (m, 2H), 7.57 (m, 1H), 7.53 (m, 1H), 7.32 (m, 2H), 7.23 (m, 2H)); ¹³C NMR (125.7 MHz, DMSO-d6) δ 180.7, 170.2, 137.2, 137.1, 129.8, 129.2, 125.6, 124.7, 123.4, 122.6, 122.0, 121.0, 120.7, 113.1, 112.5, 110.7, 108.1; LCMS m/z 317.0 [M+H]⁺.

### Example 3: Summary of Biology and Pharmacology

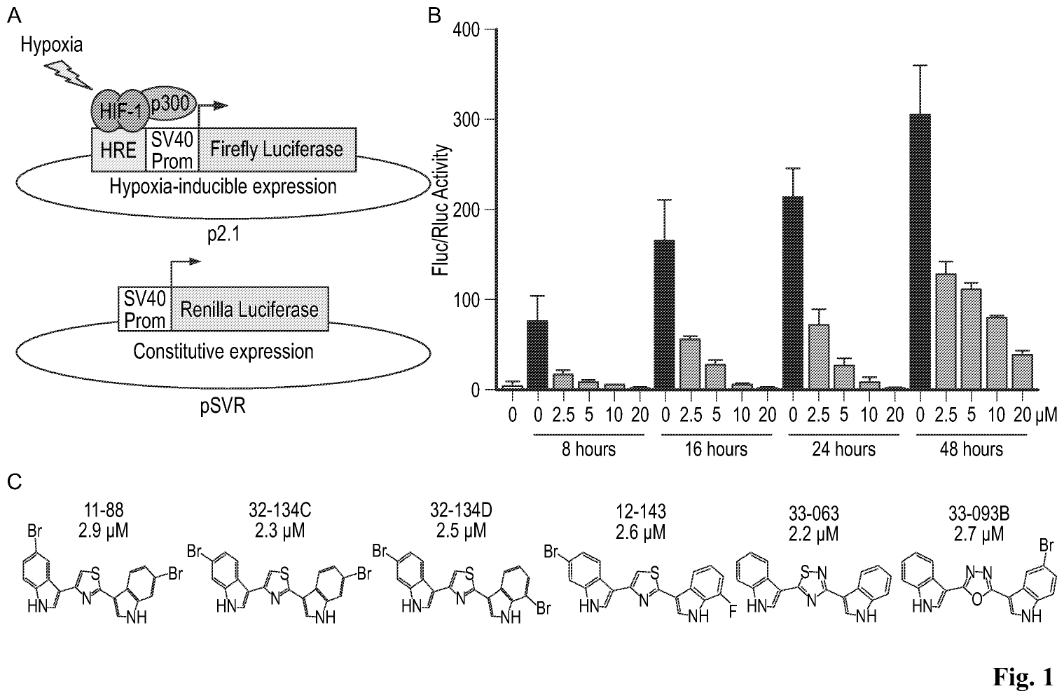

The NCI CellMiner database contains expression data on 25,698 mRNAs in 60 human cancer cell lines that were exposed to 21,770 chemical compounds (Reinhold W C, et. al., Cancer Res. 2012; 72(14):3499-3511). The CellMiner database was interrogated for compounds that induced changes in gene expression highly correlated with those induced by acriflavine, but would be structurally unrelated to acriflavine. Compounds that satisfied these criteria were analyzed in Hep3B-c1 cells (Zhang H. et. al., Proc Natl Acad Sci USA. 2008; 105(50):19579-19586), which are stably transfected with: HIF dependent reporter plasmid p2.1, in which firefly luciferase (FLuc) coding sequences are located downstream from a hypoxia response element (HRE) and a basal SV40 promoter; and control reporter pSVR, in which Renilla luciferase (RLuc) coding sequences are downstream of the basal SV40 promoter only (FIG. 1A). The FLuc/RLuc ratio in hypoxic cells is a specific measure of HIF-dependent gene expression (FIG. 1B). NSC-705870 (designated 11-88 herein), a bis-bromoindole thiazole compound that had a Pearson's correlation of 0.475 (p=1.2×10-4) with acriflavine in the CellMiner database, significantly inhibited the FLuc/RLuc ratio in hypoxic Hep3B cells with an IC₅₀ of 2.9 μM (FIG. 1C).

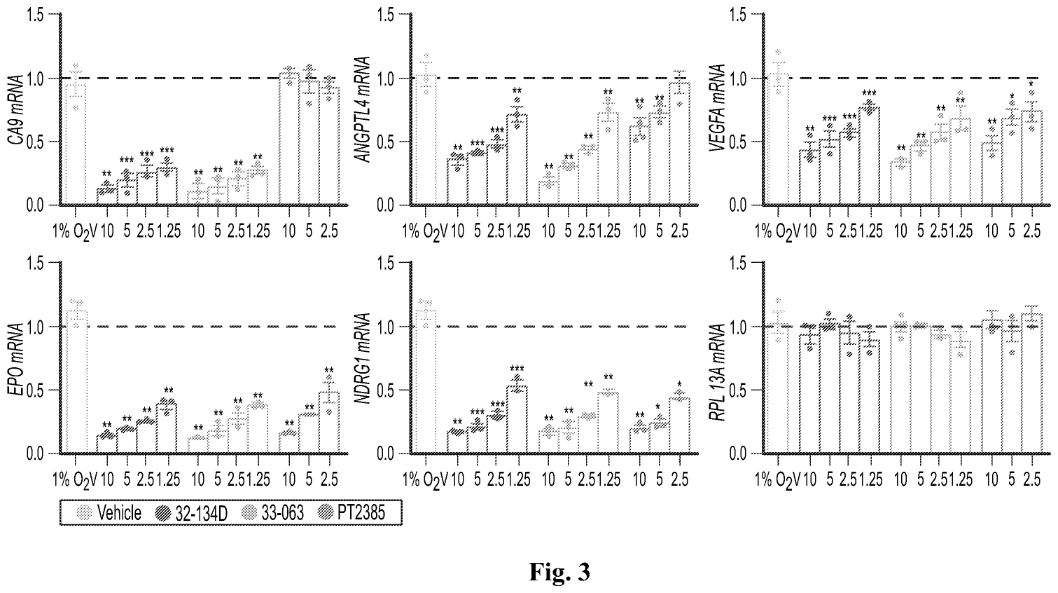

Based on hit compound 11-88, a number of analogs were prepared in which either halogen substitutions to the indole groups were altered or the central thiazole was replaced by imidazole, isoxazole, oxadiazole, oxazole, pyrazinone, pyridazine, pyrazine, pyrazole, pyridine, thiadiazole, triazine, or triazole. Among 224 analogs, 27 compounds were identified that inhibited HIF transcriptional activity with IC₅₀<3.3 μM (FIGS. 1C, 2 and Table 7). The bis-bromoindole thiazole 32-134D and the bis-indole thiadiazole 33-063 (FIGS. 1B and 1C) were further characterized with respect to endogenous HIF target gene expression in Hep3B cells. Reverse transcription (RT) and quantitative real-time PCR (qPCR) revealed that both of these compounds inhibited the hypoxia-induced expression of the following HIF target genes: CA9, which in Hep3B cells is activated by HIF-1 only; ANGPTL4 and VEGFA, which are regulated both HIF-1 and HIF-2; and NDRG1 and EPO, which are activated only by HIF-2 in Hep3B cells (FIG. 3). These data suggest that these compounds inhibit transcription mediated by either HIF-1 or HIF-2. For comparison, we tested the effect of PT2385, which inhibited EPO, NDRG1, ANGPTL4, and VEGFA expression but had no effect on CA9, which is consistent with its HIF-2-selective mechanism of action (Wallace E M et al., Cancer Res. 2016; 76(18):5491-5500). None of the compounds had any effect on RPL13A expression, which is neither hypoxia-induced nor HIF-regulated (FIG. 3).

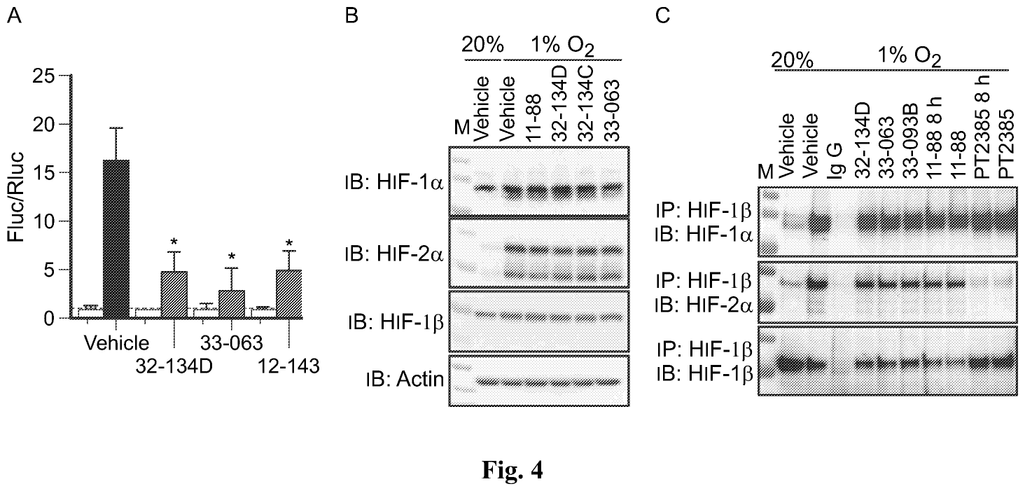

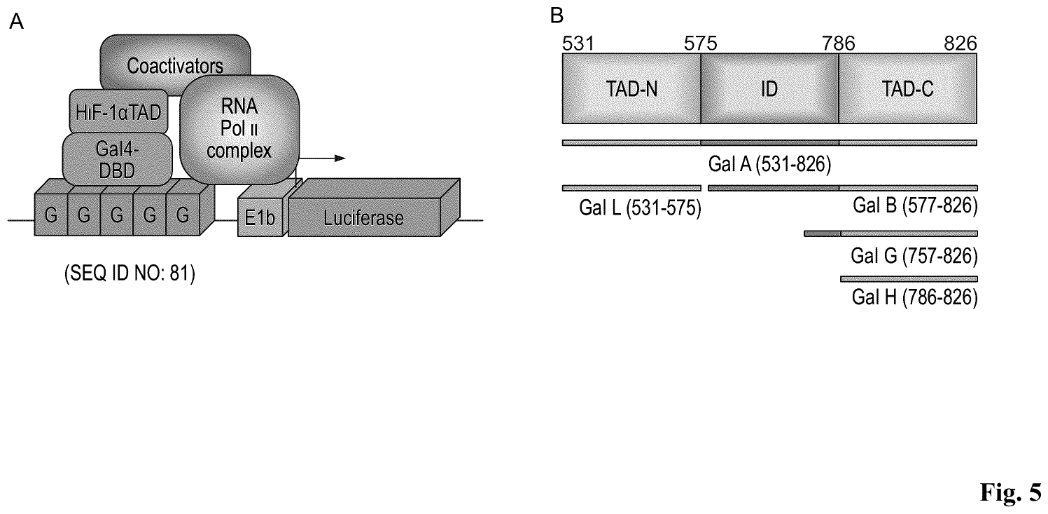

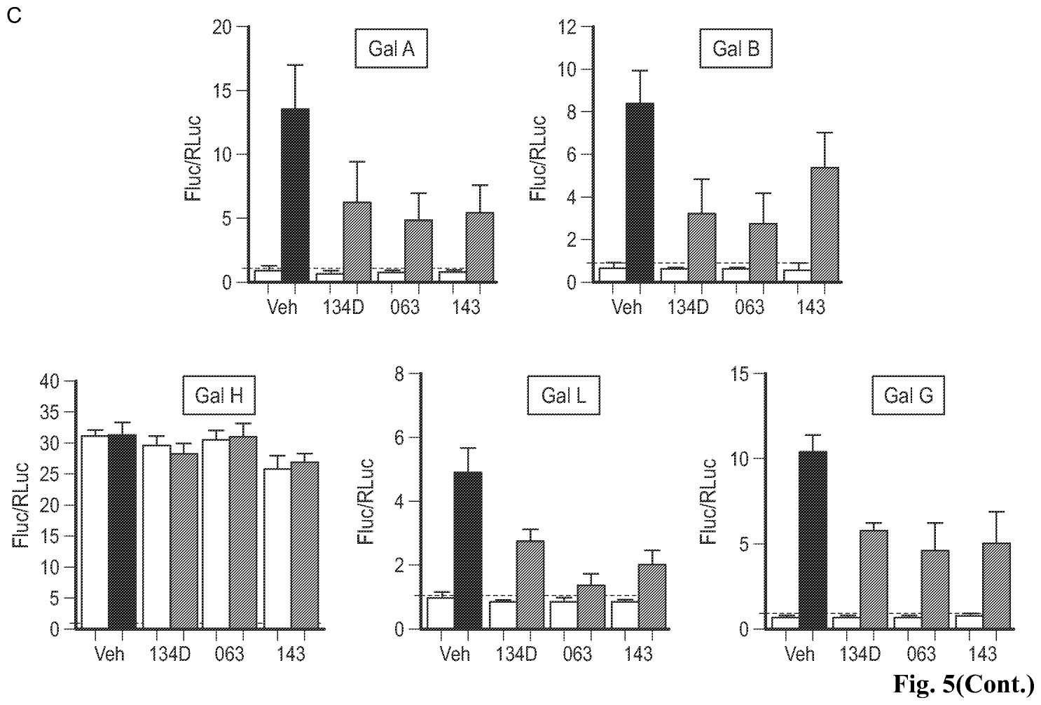

To investigate mechanism of action, the ability of several compounds to inhibit hypoxia-induced and HIF-dependent reporter gene expression was confirmed (FIG. 4A); it was found that none of the compounds tested had any effect on the expression of HIF-1α, HIF-2α or HIF-1β protein (FIG. 4B) nor did they affect HIF subunit dimerization (FIG. 4C). To test the hypothesis that the novel HIF inhibitors act by blocking HIF transactivation domain (TAD) function, a reporter system (Jiang B H et al., J Biol Chem. 1997; 272(31):19253-19260) was utilized consisting of plasmid pG5-E1b-FLuc, which contains five binding sites for the yeast GAL4 transcription factor upstream of a basal adenovirus E1b promoter and FLuc coding sequences (FIG. 5A), and pGal expression vectors, which encode fusion proteins consisting of the GAL4 DNA binding domain and HIF-1α amino acid residues 531-826 encoding the complete TAD (Gal A) or subfragments thereof (Gal B, G, H, and L) (FIG. 5B). Expression of Gal A, B, G, and L led to hypoxia-induced transactivation of FLuc, as previously described (Jiang B H et al., J Biol Chem. 1997; 272(31):19253-19260). In contrast, fusion protein Gal H mediated constitutive transactivation because it lacks amino acids 757-785 in the inhibitory domain that are required for interaction with FIH-1 (Mahon P C et al. Genes Dev 2001; 15(20):2675-2686), which hydroxylates asparagine 803 when O₂ is available, thereby blocking recruitment of the coactivator p300 (Lando D et al., Genes Dev. 2002; 16(12):1466-1471). Remarkably, all three inhibitors blocked hypoxia-induced FLuc expression mediated by Gal A, B, G, or L, but had no effect on the constitutive activity of Gal H (FIG. 5C), which eliminates the possibility that the compounds directly inhibit p300 coactivator function.

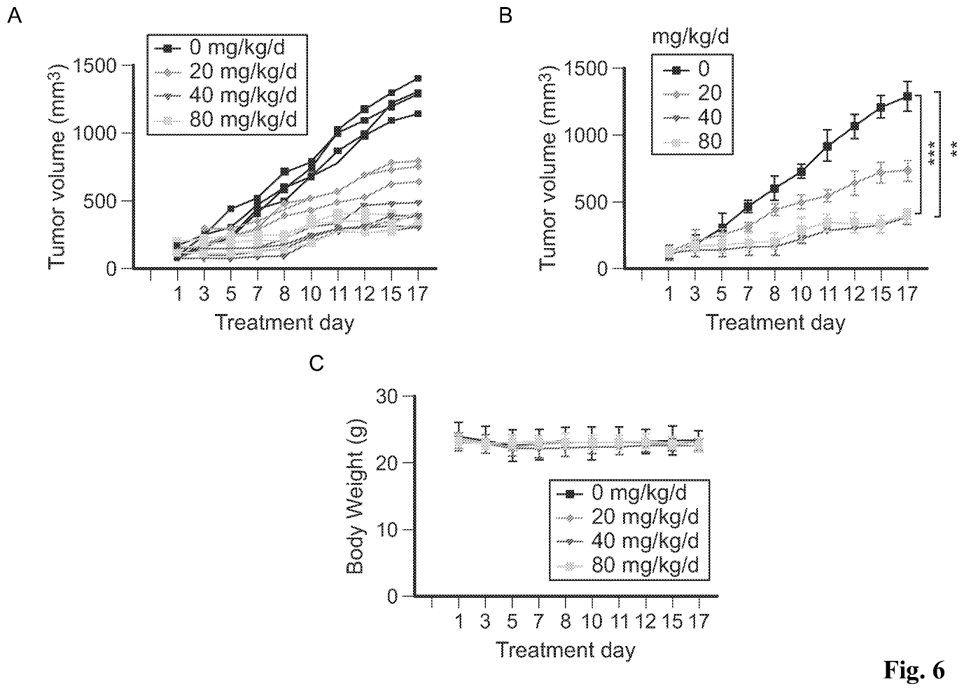

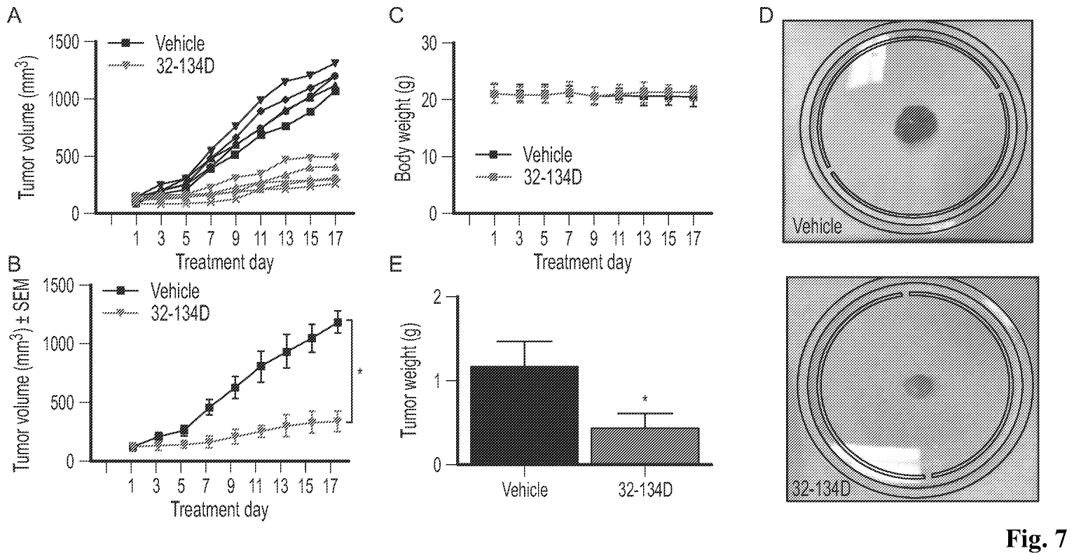

Effects of 32-134D on Hep3B human HCC tumor xenografts. To analyze the effect of HIF inhibitor 32-134D on tumor xenograft growth, Hep3B cells were injected subcutaneously into immunodeficient mice and when tumors reached a volume of 150 mm3 (designated treatment day 1), the mice were treated with vehicle or 32-134D by daily intraperitoneal injection. Partial growth inhibition was observed at 20 mg/kg and maximal growth inhibition was observed at 40 and 80 mg/kg (FIGS. 6A-B). Treatment with 32-134D for 17 days had no effect on mouse body weight (FIG. 6C), appearance or behavior. We treated additional mice with 32-134D and confirmed inhibition of tumor growth without effects on body weight (FIGS. 7A-C). The mass of tumors harvested from 32-134D-treated mice was significantly decreased (FIG. 7D) and demonstrated pallor in comparison to the bloody appearance of tumors from vehicle-treated mice (FIG. 7E), suggesting effects on tumor vascularization.

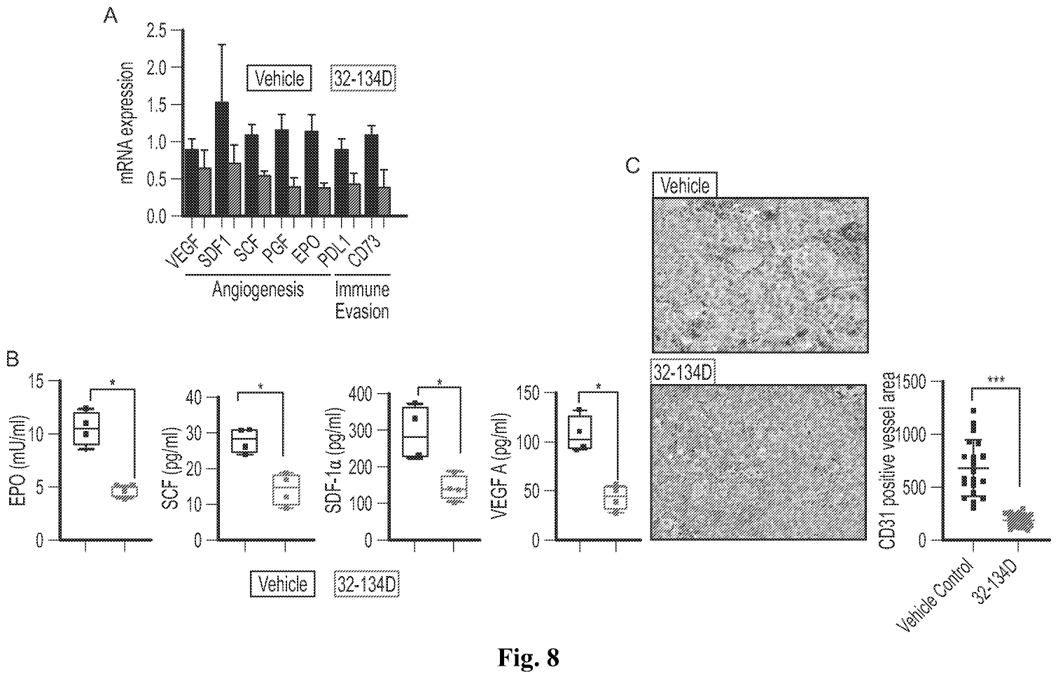

Analysis of tumor RNA revealed a significant decrease in the expression of mRNAs encoding: (i) angiogenic growth factors, including stem cell factor (SCF; also known as Kit ligand [KITLG]), placental growth factor (PGF), and erythropoietin (EPO); (ii) proteins mediating immune evasion (CD73, PDL1); and (iii) proteins with effects on both angiogenesis and immunity, including vascular endothelial growth factor A (VEGFA) and stromal-derived factor 1 (SDF1; also known as CXCL12) in response to 32-134D treatment (FIG. 8A). Whole tumor lysates were subjected to ELISA, which revealed significantly decreased expression of EPO, SCF, SDF1 and VEGFA protein in tumors from mice treated with 32-134D (FIG. 8B) Immunohistochemistry using an antibody against CD31, which is expressed by vascular endothelial cells, demonstrated markedly decreased blood vessel area in tumors from 32-134D-treated mice (FIG. 8D). Thus, treatment with 32-134D potently inhibits human HCC tumor xenograft growth and angiogenesis by inhibiting the expression of multiple HIF target genes.

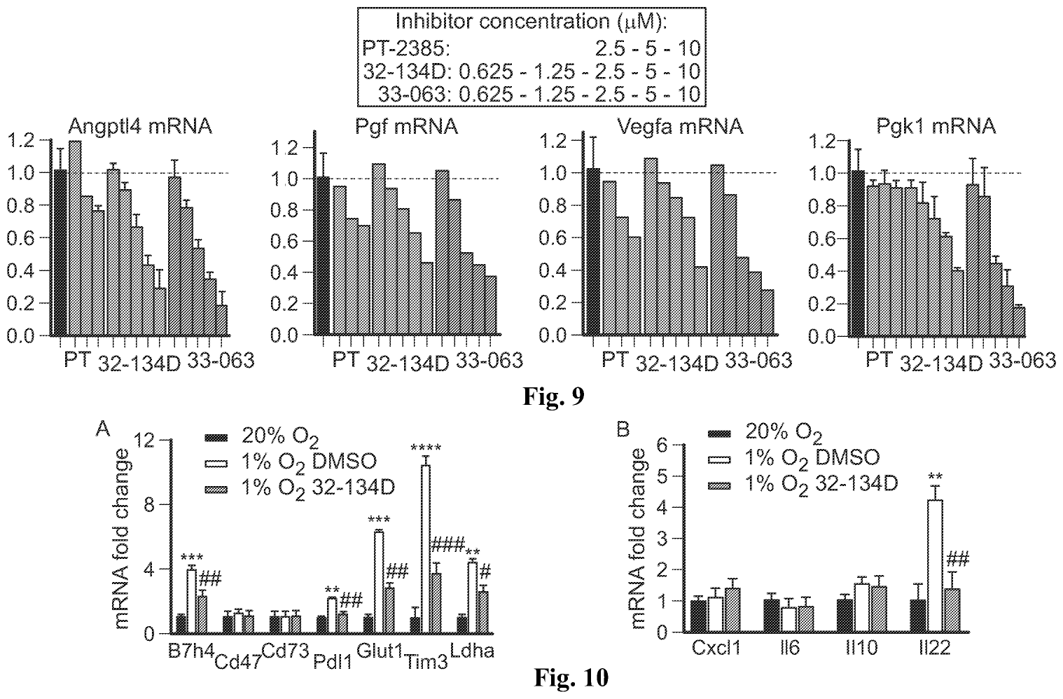

Effects of 32-134D on Hepa1-6 mouse HCC cells and tumors. Treatment of hypoxic Hepa1-6 mouse HCC cells with 33-063 or 32-134D revealed significant dose-dependent inhibition of hypoxia-induced expression of mRNA encoding Angpt14, Pgf, Vegfa and Pgk1 (the glycolytic enzyme phosphoglycerate kinase 1) (FIG. 9). PT2385 was a less potent inhibitor of Angpt14, Pgf, and Vegfa mRNA expression and had no effect on expression of Pgk1 mRNA, which is mediated by HIF-1 only. 32-134D treatment also blocked the hypoxia-induced expression of Glut1 (glucose transporter 1) and Ldha (the glycolytic enzyme lactate dehydrogenase A) (FIG. 10A). Thus, 32-134D inhibits the ability of hypoxic HCC cells to increase O₂ delivery by stimulating angiogenesis (Angpt14, Pgf, Vegfa) or stimulates anaerobic metabolism by increasing glycolytic flux (Glut1, Pgk1, Ldha).

Analysis of genes that are hypoxia-induced by HIFs in breast cancer and promote immune evasion (Samanta D et al., Proc Natl Acad Sci USA 2018;115(6):E1239-E1248) revealed that Cd47 and Cd73 were not hypoxia-induced in Hepa1-6 cells, whereas Pdl1 mRNA was hypoxia-induced in vehicle-treated but not in 32-134D-treated cells (FIG. 10A). However, B7h4 and Tim3 mRNAs, which, like Pdl1, encode immune checkpoint receptors that are HIF-regulated (Jeon Y K et al., Biochem Biophys Res Commun. 2015; 459:277-2833; Koh H S et al., Nat Commun. 2015; 6:6340) and associated with patient mortality in HCC (Shrestha R et al., Front Oncol. 2018; 8:269), were hypoxia-induced in vehicle but not in 32-134D-treated cells (FIG. 10A).

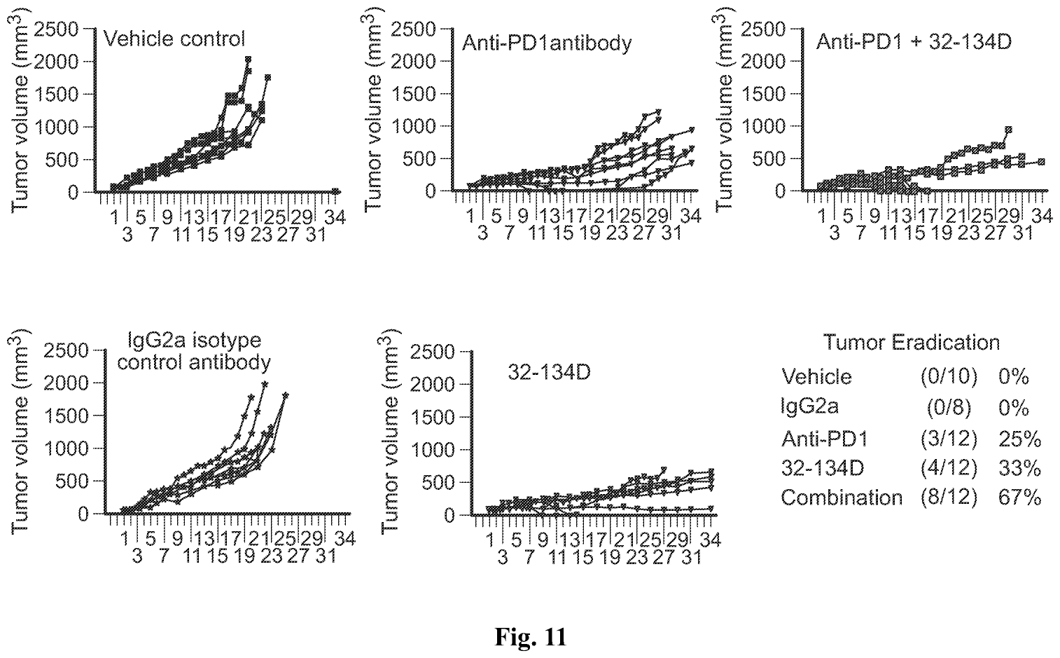

To test whether 32-134D inhibits HCC growth in an immunocompetent mouse model, Hepa1-6 HCC cells were injected into the flank of syngeneic C57L mice (Darlington G J et al., J Natl Cancer Inst. 1980; 64(4):809-819). When tumors became palpable (treatment day 1), mice received daily intraperitoneal injections of 32-134D (40 mg/kg/d) versus vehicle control, or anti-Pd1 antibody versus IgG2a isotype control (200 g on days 1, 4, 7, 10, and 16), or both 32-134D and anti-Pd1. Tumors grew very rapidly in all mice treated with vehicle or IgG2a, necessitating euthanasia on or before day 24 (FIG. 9). Tumors regressed in 5 out of 12 mice treated with anti-Pd1, but two tumors recurred after the last antibody treatment (on day 16) for a tumor eradication rate of only 25%. Treatment with 32-134D resulted in a marked inhibition of tumor growth in all mice and tumor eradication was achieved in 4 of 12 mice. Among mice treated with the combination of 32-134D and anti-Pd1, tumor growth was markedly decreased and tumor eradication occurred in 8 of 12 mice, with no recurrences after antibody treatment was discontinued (FIG. 11). Thus, addition of 32-134D increased the percentage of mice with a durable complete response to anti-Pd1 immune checkpoint blockade from 25% to 67%.

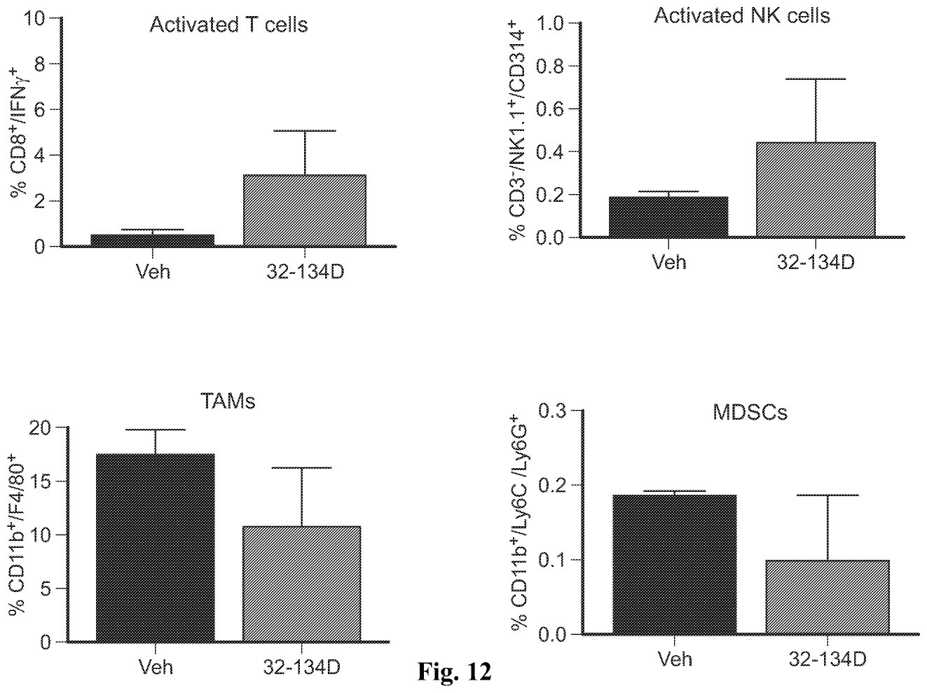

To analyze the mechanism by which 32-134D increased the response to anti-Pd1 therapy, tumor-bearing mice were treated with 32-134D or vehicle, and immune cell populations in well-established 200-mm³ tumors were analyzed by flow cytometry. Within 8 days, HIF inhibitor therapy significantly increased the percentage of Cd8⁺ Ifng⁺ activated cytotoxic T lymphocytes (CTLs) and Nk1.1⁺Cd3⁻Cd314⁺ activated natural killer (NK) cells, which are the two immune cell populations that are critical effectors of anti-tumor immunity (FIG. 12). In contrast, there was a decrease in the percentage of Cd11b⁺F4/80⁺ tumor-associated macrophages and Cd11b⁺Ly6g⁺Ly6c⁻ myeloid-derived suppressor cells (FIG. 12), which are immune cell populations that are critical for immunosuppression in HCC (Yuen V W et al., J C/in Invest. 2020; 130(10):5052-5062).

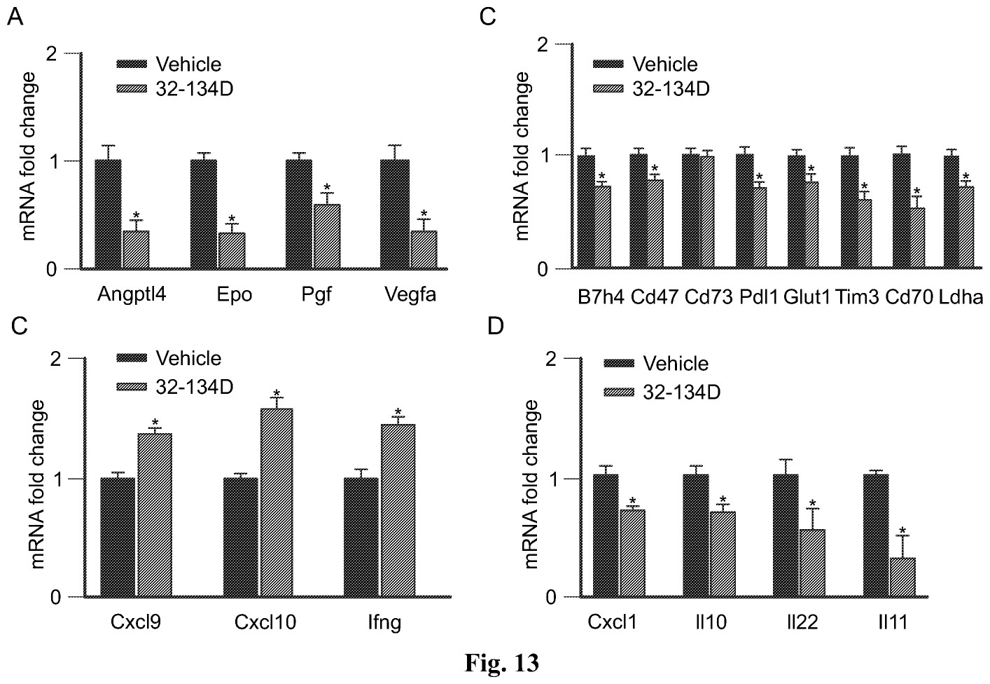

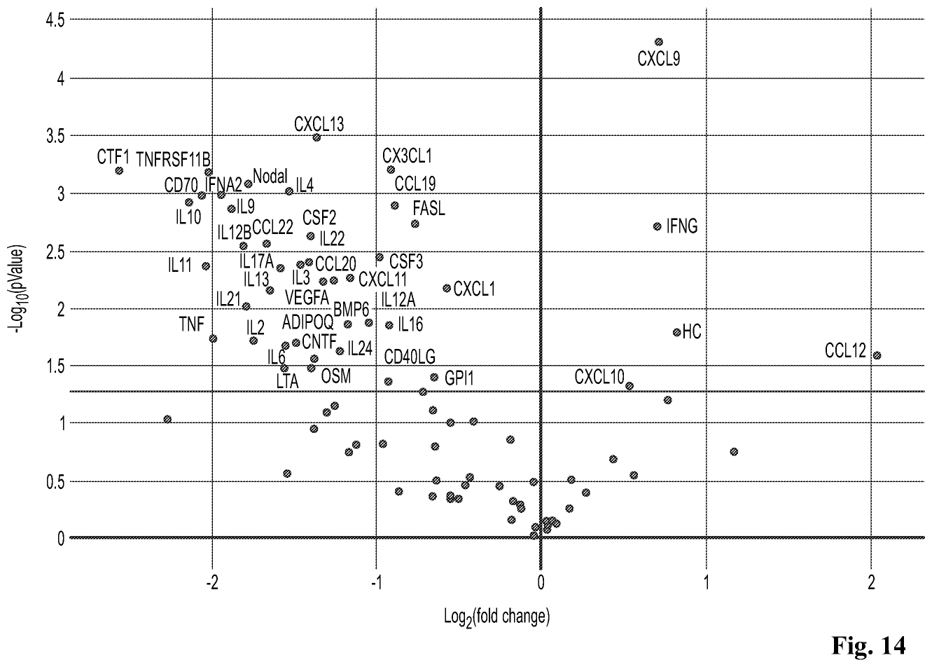

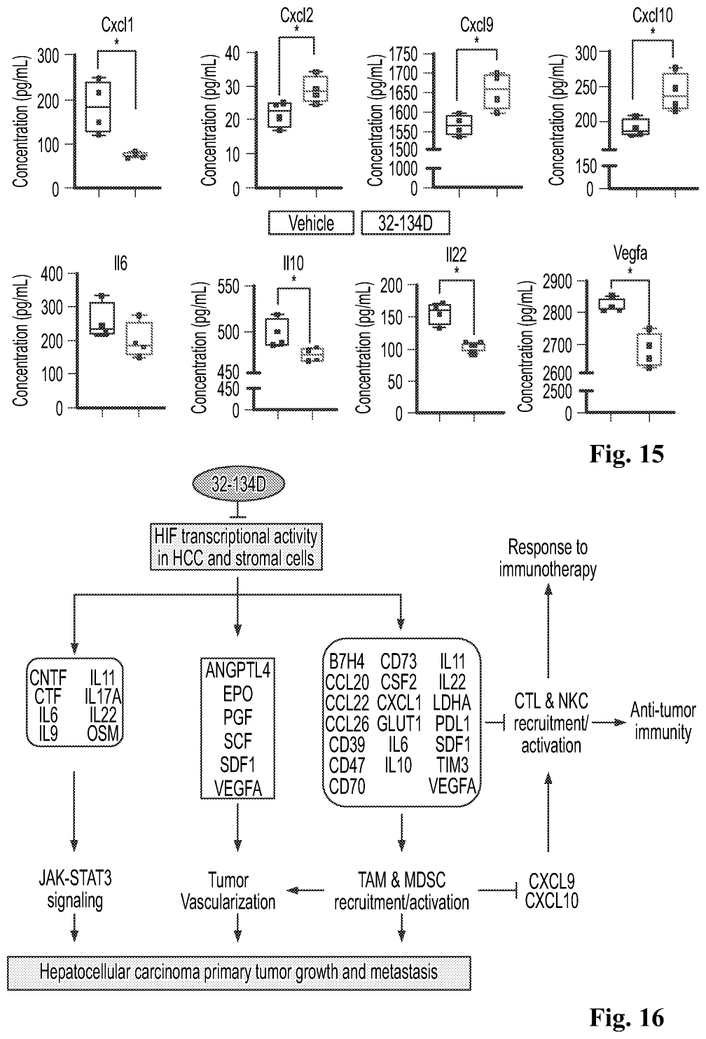

Analysis of gene expression in tumor tissue by RT-qPCR revealed that 32-134D treatment significantly decreased the expression of multiple mRNAs encoding angiogenic factors (Angpt14, Epo, Pgf, Vegfa; FIG. 13A) and proteins mediating immunosuppression (B7h4, Cd47, Cd70, Cd73, Glut1, Ldha, Pdl1, Tim3, Vegfa; FIGS. 13A-B), whereas 32-134D treatment increased the expression of Cxcl9 and Cxcl10, two cytokines that are critical for recruitment of CTLs and NK cells (FIG. 13C). To extend our characterization of the tumor immune microenvironment, we utilized an RT-qPCR array to analyze 84 cytokines and chemokines, which revealed decreased expression of 40 mRNAs, including those encoding the immunosuppressive cytokines Cxcl1, Il3, Il4, Il6, Il10, Il12A, Il12B, Il13 and Vegfa, and increased expression of 5 mRNAs, including Cxcl9 and Cxcl10, in tumors from 32-134D-treated mice (FIG. 14 and Table 1). To extend these mRNA data, protein expression was quantified by performing ELISAs on tumor lysates, which confirmed that tumors from 32-134D-treated mice contained significantly increased levels of Cxcl9 and Cxcl10, which promote anti-tumor immunity, as well as significantly decreased levels of Cxcl1, Il10 and Vegfa (FIG. 15), which promote establishment of an immunosuppressive tumor microenvironment.

IL22 mRNA expression was induced by hypoxia and inhibited by 32-134D in both cultured Hepa1-6 cells (FIG. 10B) and expression of both IL22 mRNA (FIG. 13D), and protein (FIG. 15) in Hepa1-6 tumors were inhibited by 32-134D. In contrast, decreased Cxcl1, Il6 and Il10 mRNA levels were observed in tumor tissue from 32-134D-treated mice (FIG. 13D) but not in cultured Hepa1-6 cells exposed to 32-134D (FIG. 10B), which suggests that in vivo 32-134D inhibited the expression of these latter mRNAs in immune or other stromal cell types, where HIFs are also known to play critical roles (Kumar V et al., Immunology. 2014; 143(4):512-519; Noman M Z et al., Oncoimmunology. 2015;3(12):e954463; Palazon A et al., Immunity. 2014; 41(4):518-528), rather than in HCC cells.

The effects of HIF inhibitors on gene expression are consistent with therapeutic benefit. Treatment with 32-134D led to: (i) decreased expression of Ca9, Cxcl1, Epo, Ldha, Pgf, Scf/Kitlg, and Glut1/Slc2a1 mRNA, which are all associated with HCC patient mortality; and (ii) increased expression of Ccl12/CCL2, Cxcl2, Cxcl9, Cxcl10, and He/C5 mRNA, which are all associated with HCC patient survival (Table 2). Taken together, the data presented above demonstrate that treatment of HCC-bearing mice with the HIF inhibitor 32-134D significantly impairs tumor vascularization, markedly alters the tumor immune microenvironment in favor of anti-tumor immunity, and blocks key signal transduction pathways driving cancer progression (FIG. 16), thereby providing a broad molecular and cellular foundation for the increased tumor eradication observed in mice treated with 32-134D and anti-Pd1 antibody.

### HIF Inhibitor 32-134D Effectively Inhibits HIF Accumulation and Expression of HIF-Regulated Genes In Vitro.

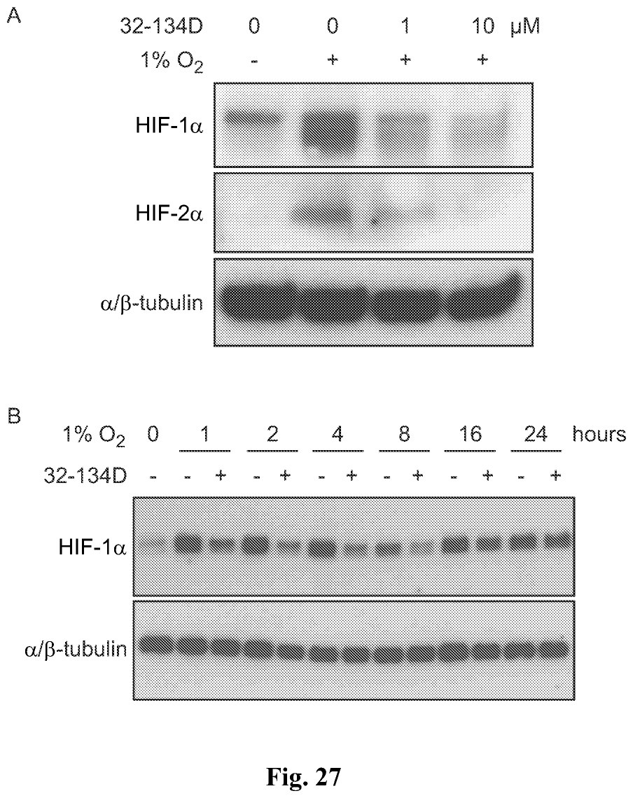

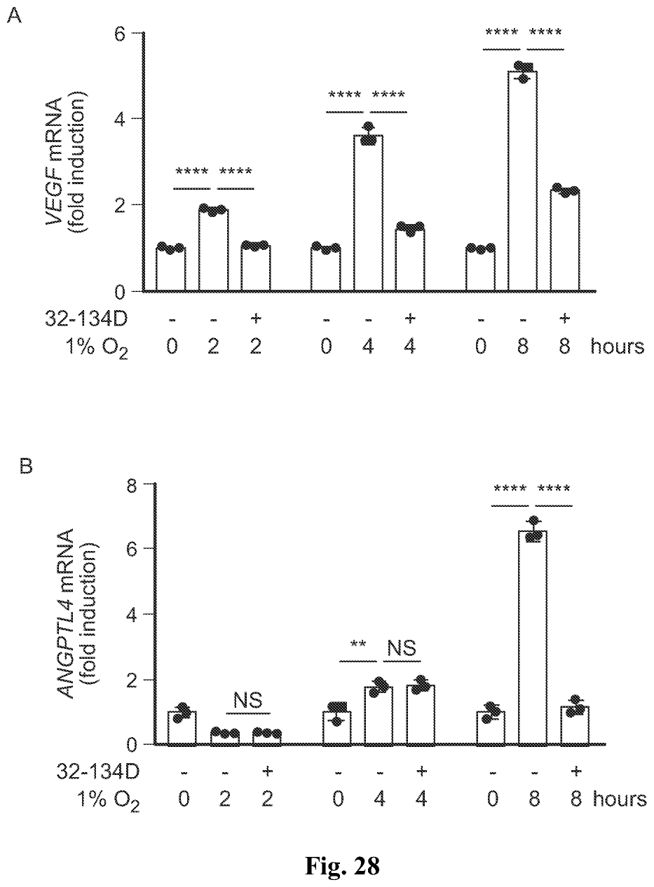

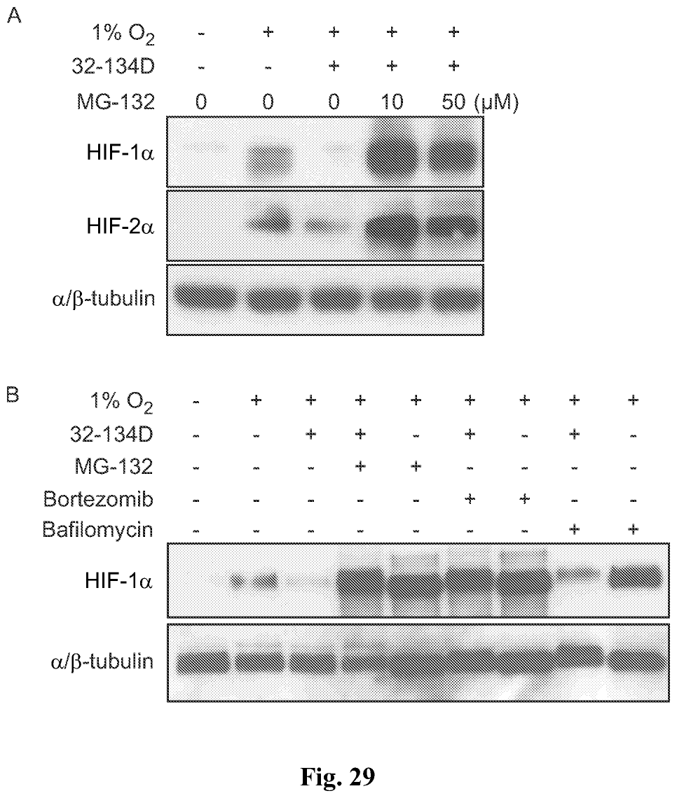

It has previously been reported that the cells in the inner nuclear layer of the retina that express VEGF in response to hypoxia/ischemia are Müller glial cells (X. Xin et al., Proc Natl Acad Sci USA 110, E3425-3434 (2013)). Treatment of the immortalized human Müller cell line, MIO-M1, with as little as 1 μM of 32-134D resulted in decreased HIF-1α and HIF-2α protein accumulation in response to hypoxia (FIG. 27A). The effects of 32-134D on HIF-1α accumulation persisted up to 16 hours after treatment of cultured cells (FIG. 27B). This, in turn, resulted in a potent inhibition of the mRNA expression of VEGF and ANGPTL4 (FIGS. 28A and 28B). Pre-treatment with the proteasome inhibitor MG-132 prevented the ability of 32-134D to block HIF-1α and HIF-2α protein accumulation (FIG. 29A), indicating that 32-134D induced proteasomal of HIF-α subunits in MIO-M1 cells, as previously described for Hep3B human hepatocellular carcinoma cells (S. Salman et al., J Clin Invest 132 (2022)). These results were corroborated using another proteasome inhibitor, bortezomib (FIG. 29B). Conversely, bafilomycin, an inhibitor of lysosomal acidification did not prevent 32-134D from inhibiting HIF-1α accumulation in response to hypoxia (FIG. 29B). Collectively, these results demonstrate that 32-134D promotes the proteasome-dependent degradation of HIF-1α and HIF-2α protein despite the presence of hypoxia.

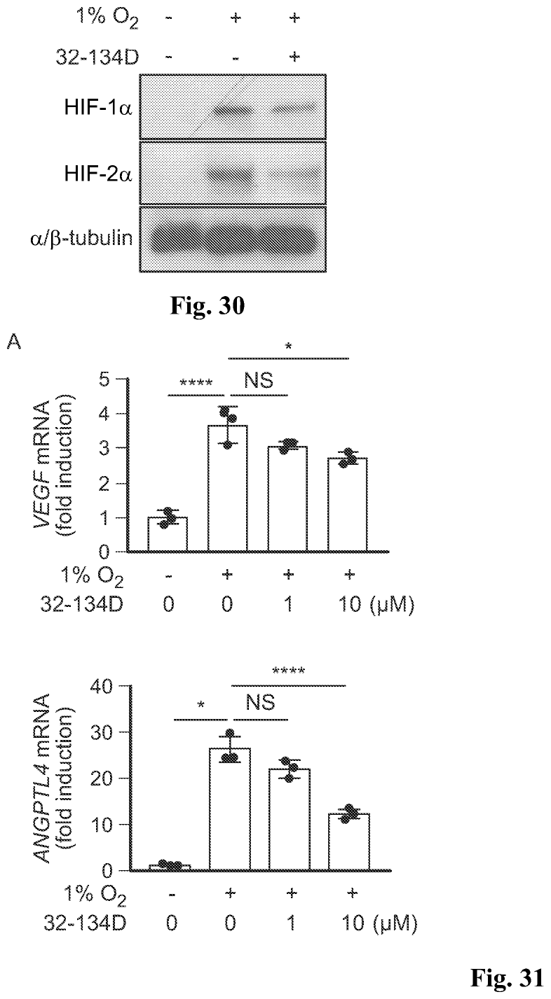

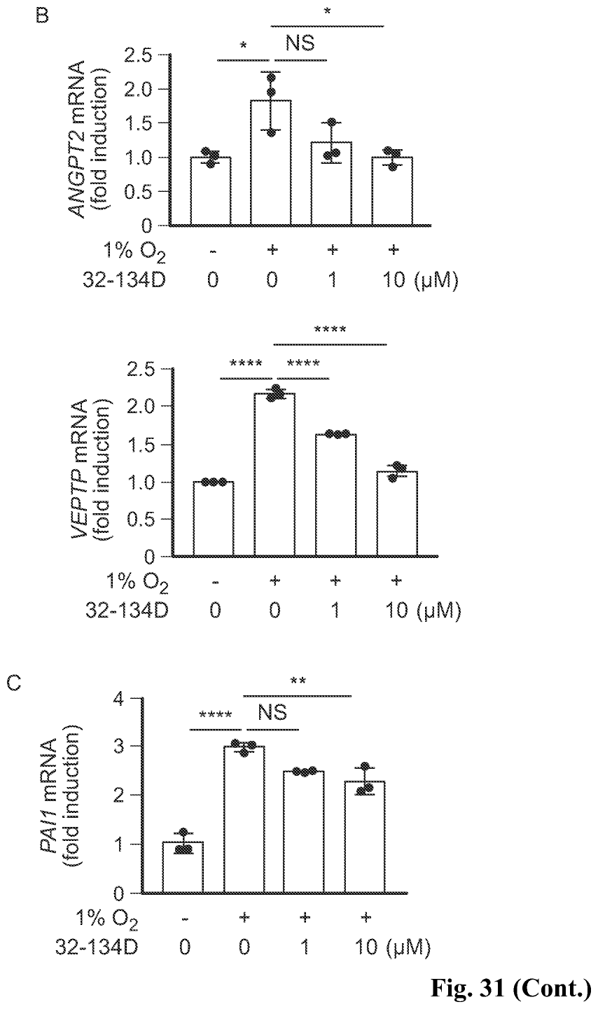

Vascular endothelial cells also secrete angiogenic mediators that contribute to the progression of diabetic eye disease (Y. Qin et al., Sci Adv 8, eabm1896 (2022)). Exposure of human umbilical vein endothelial cells (HUVECs) to hypoxia induced HIF-1α and HIF-2α protein accumulation, which was inhibited by 32-134D (FIG. 30). Accordingly, 32-134D effectively inhibited hypoxia-induced VEGF and ANGPTL4 mRNA expression (FIG. 31A) similar to what was observed with MIO-M1 cells. Of note, ANGPT2 and VE-PTP, two emerging targets for the treatment of diabetic eye disease, are both expressed specifically by vascular cells (G. Fachinger, et al., Oncogene 18, 5948-5953 (1999); P. C. Maisonpierre et al., Science 277, 55-60 (1997)). Treatment of HUVECs with hypoxia resulted in increased expression of ANGPT2 and VEPTP mRNA, which was inhibited by 32-134D (FIG. 31B). Similar results were obtained for the recently identified HIF-2-dependent vascular cell-specific paracrine angiogenic mediator, PAI-1 (Y. Qin et al., Sci Adv 8, eabm1896 (2022)); FIG. 31C). Collectively, these results demonstrate that 32-134D can effectively inhibit the hypoxia-induced accumulation of both HIF-1α and HIF-2α, and the expression of the key angiogenic genes they regulate, in multiple retinal cell types.

### 32-134D Inhibits HIF Accumulation and Expression of HIF-Regulated Genes in Human-Inducible Pluripotent Stem Cell-Derived 3-Dimensional Retinal Organoids.

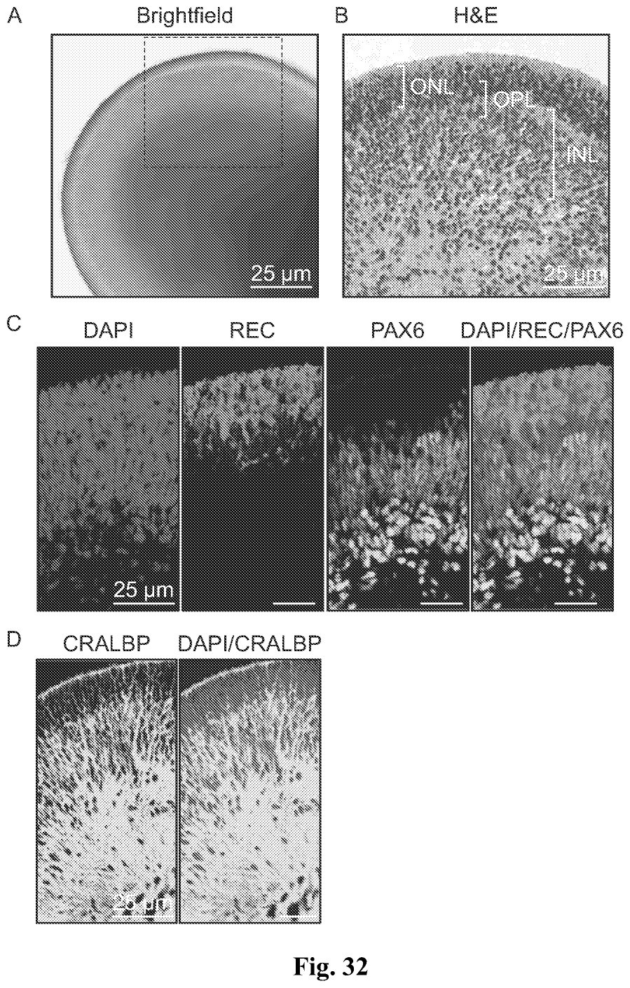

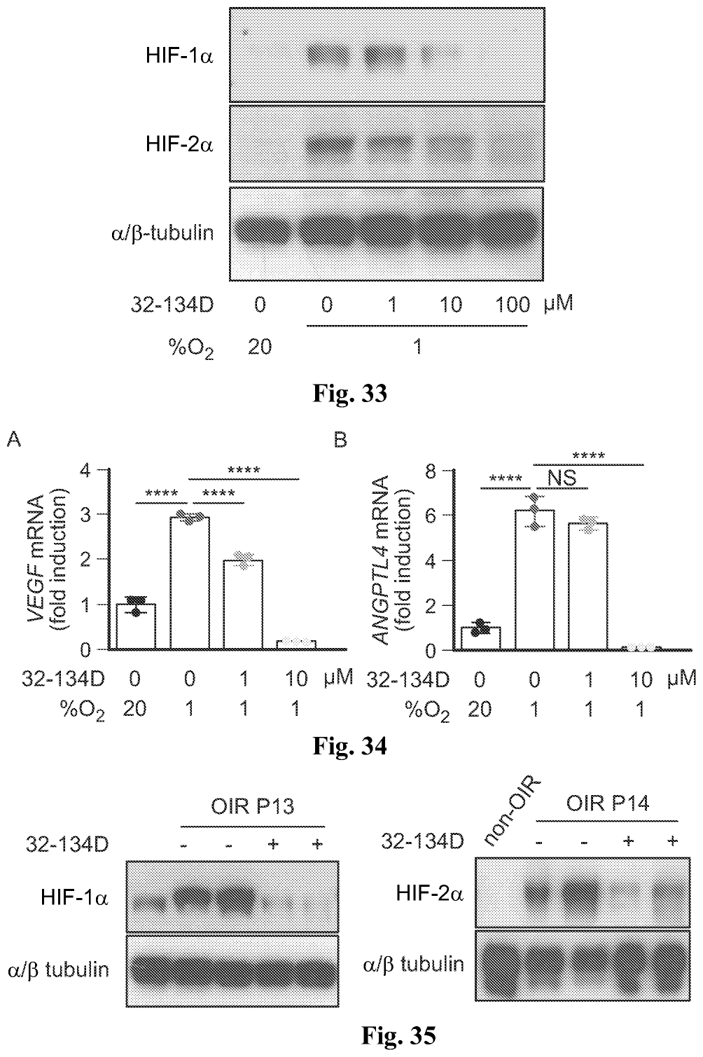

To assess the therapeutic potential of 32-134D for diabetic patients, human induced pluripotent stem cell (hiPSC)-derived 3-dimensional (3D) retinal organoids were treated with 32-134D. It has been previously reported that hiPSC-derived 3D retinal organoids cultured under hypoxic conditions (1% O₂) behave similar to ischemic human retinal tissue with increased accumulation of both HIF-1α and HIF-2α (J. Zhang et al., J Clin Invest 131 (2021)). By 120 days of differentiation (D120), the inner and outer retinal layers are clearly defined (FIGS. 32A and 32B) and contain the precursors of the major retinal cell types, including outer retina photoreceptors (expressing recoverin), few newly differentiating bipolar cell precursors (lacking expression of recoverin and Pax6), amacrine cells (expressing high levels of Pax6), as well as Müller cells (expressing CRALBP; FIGS. 32C and 32D). Culturing D120 retinal organoids in 1% O₂ resulted in increased HIF-1α and HIF-2α protein accumulation (FIG. 33) and increased expression of HIF-regulated vasoactive mediators (FIGS. 34A and 34B). Treatment of hiPSC-derived 3D retinal organoids with 32-134D effectively prevented accumulation of HIF-1α and HIF-2α protein (FIG. 33) and expression of HIF-regulated vasoactive mediators (FIGS. 34A and 34B).

### Systemic Administration of a Well-Tolerated Dose of 32-134D Inhibits HIF and HIF-Regulated Gene Expression and Effectively Treats Retinal Vascular Disease in Mice.

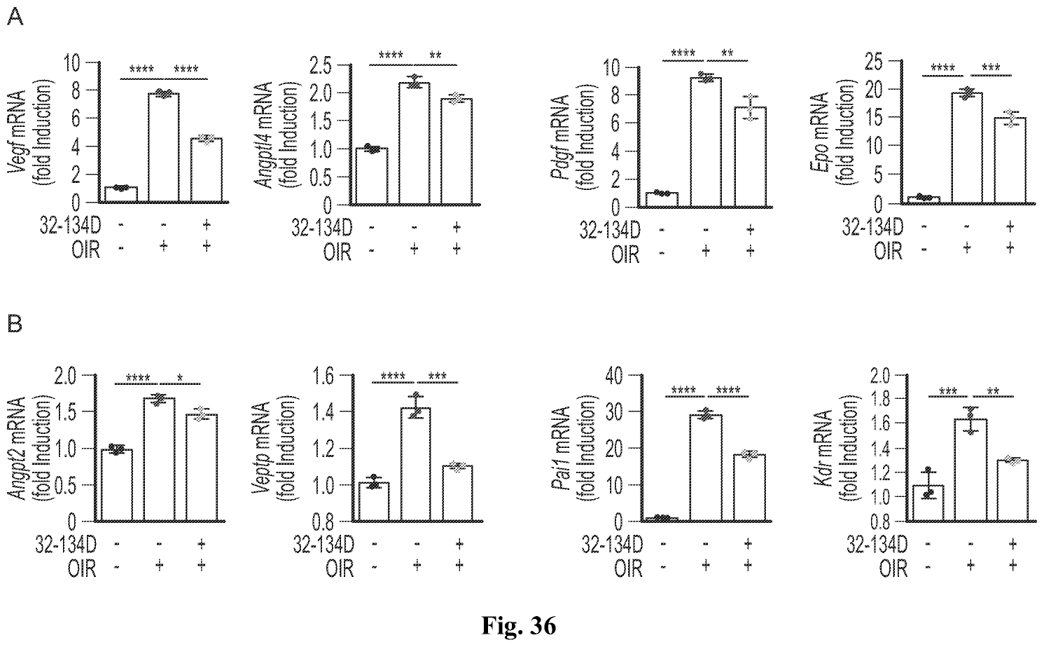

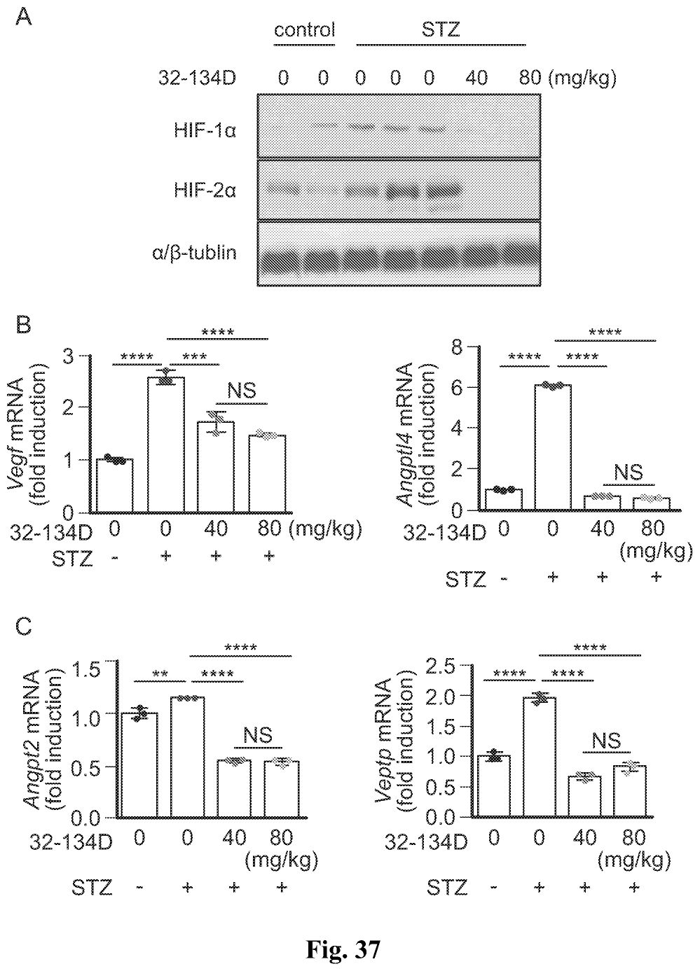

OIR mice treated with a single IP injection with as little as 20 mg/kg of 32-134D demonstrated a reduction in HIF-1α and HIF-2α protein accumulation (FIG. 35) at their respective peak expression at P13 and P14, respectively (J. Zhang et al., J Clin Invest 131 (2021)). Treatment of mice with five consecutive (P12-P16) IP injections with 20 mg/kg 32-134D resulted in a marked reduction in the mRNA expression of key HIF-regulated vasoactive mediators (FIG. 36A), including those specifically expressed by vascular cells (FIG. 36B). Similar results were observed in STZ mice that were hyperglycemic for 6 months and treated with 5 consecutive days of IP injections with 40 or 80 mg/kg 32-134D (FIG. 37).

Intraocular Administration 32-134D does not Affect Retinal Function.

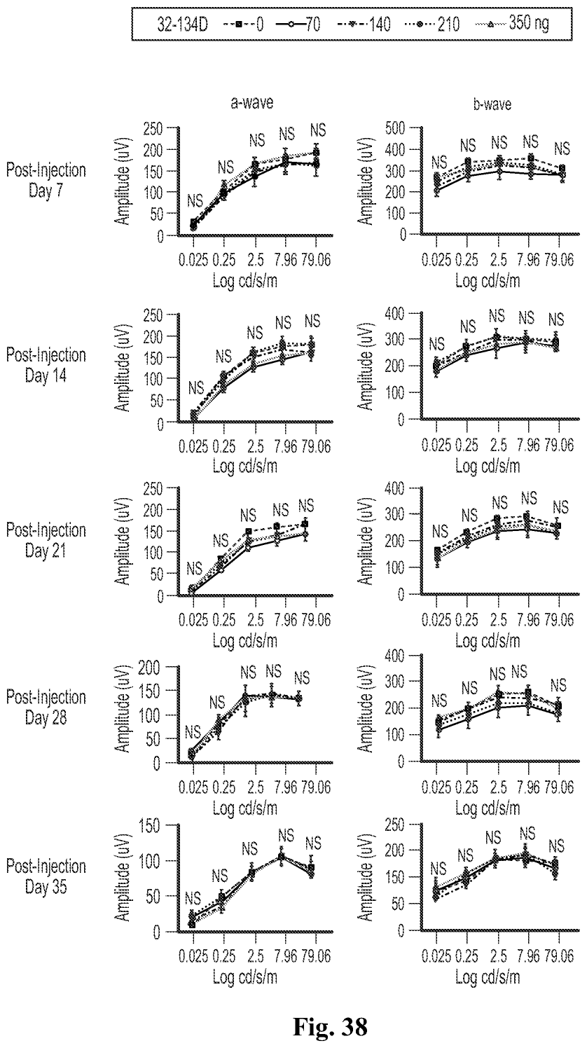

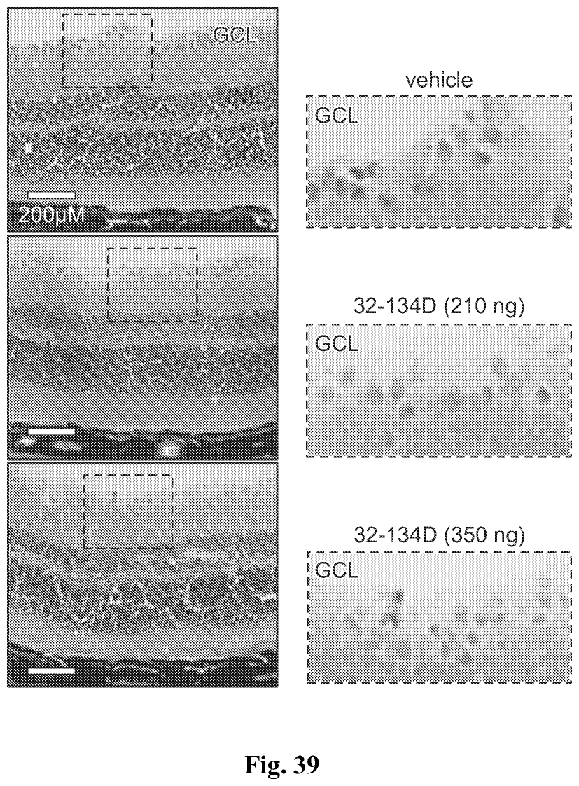

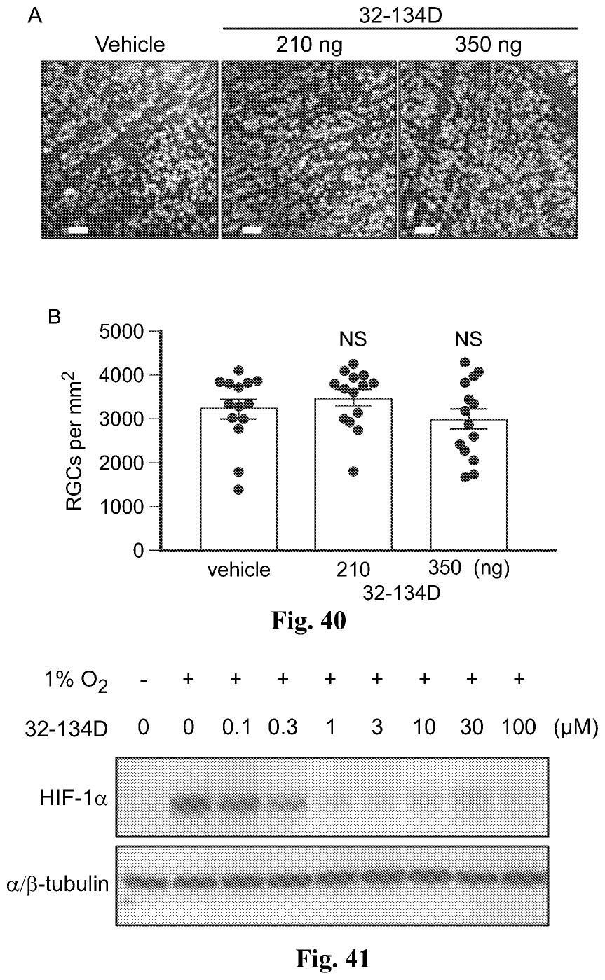

To reduce the risk of systemic side effects while optimizing the delivery of drugs to retinal tissue, therapies for ocular vascular disease are often administered by intravitreal injection. Therefore, it was assessed whether 32-13D causes retinal toxicity following intraocular administration. To this end, ERGs up to 5 weeks were examined following a single intraocular injection of increasing doses (70, 140, 210, and 350 ng) of 32-134D. There was no ERG evidence of retina toxicity at any of the doses of 32-134D tested (FIG. 38). Close examination of the eyes from mice treated with intraocular administration of 32-134D revealed normal histology including a normal appearing GCL (FIG. 39) 35 days following treatment with an intraocular injection of up to 350 ng of 32-134D. Quantitation of RGCs on retinal flat mounts demonstrated that the number of RGCs was unaffected by administration of 32-134D at the highest doses tested (FIG. 40).

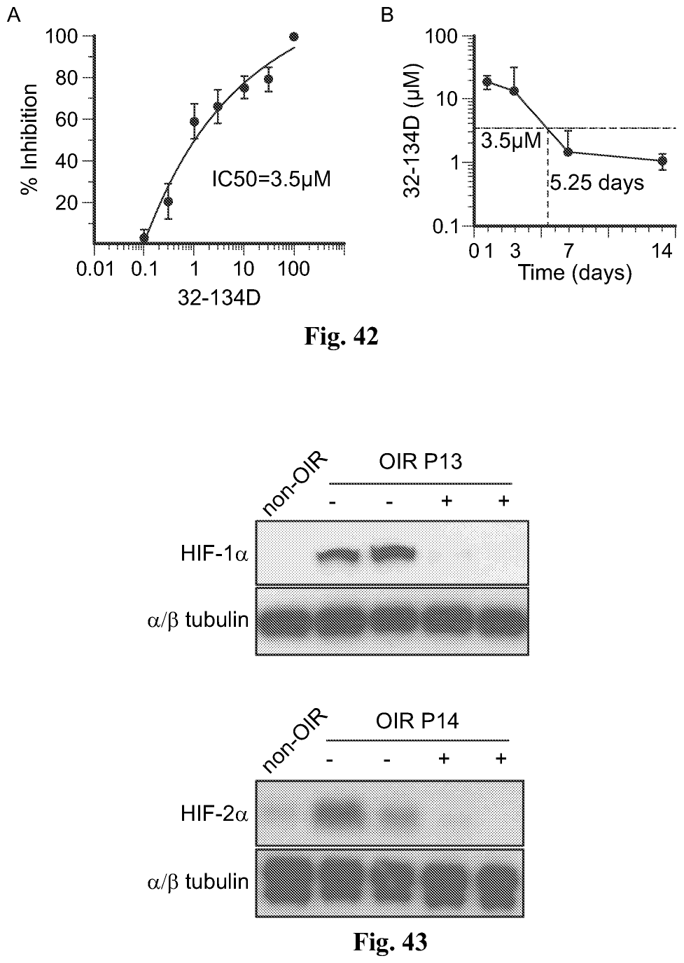

To determine the appropriate dosing for intraocular administration of 32-134D in mice, the half maximal inhibitory concentration (IC₅₀) in MIO-M1 cells was determined. Quantitation of immunoblots of HIF-1α protein accumulation in 32-134D-treated MIO-M1 cells cultured in hypoxia (FIG. 41) demonstrated an IC₅₀ of 3.5 μM (FIG. 42A). Pharmacokinetic analysis of 32-134D in the neurosensory retina following intraocular administration was determined by liquid chromatography and tandem mass spectrometry (LC-MS/MS) to quantify the concentration of 32-134D in retinal tissue over 14 days following a single 70-ng intraocular injection of 32-134D (see Methods of all parameters). The maximum concentration (Cₘₐₓ) achieved was 19.1 nmol/g at 1 day 1 with apparent monoexponential decline. The total exposure (area under the curve or AUCₗₐₛₜ) was 72.3 nmol*day/g. The concentration of 32-134D in the neurosensory retina exceeded the calculated in vitro IC₅₀ of 3.5 μM for 5.25 days (FIG. 42B). The terminal half-life (T_{1/2}) was not reportable due to the poor correlation coefficient (r²=0.35).

### Intraocular Administration of 32-134D Reduces Retinal Neovascularization and Vascular Hyper-Permeability in Mouse Models for Diabetic Eye Disease.

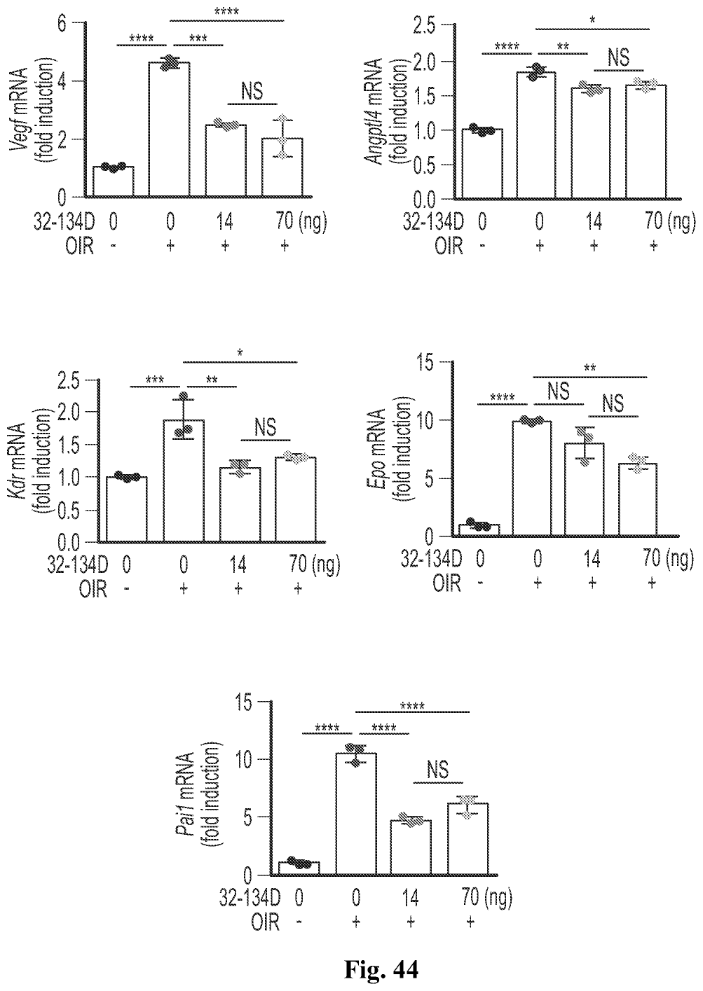

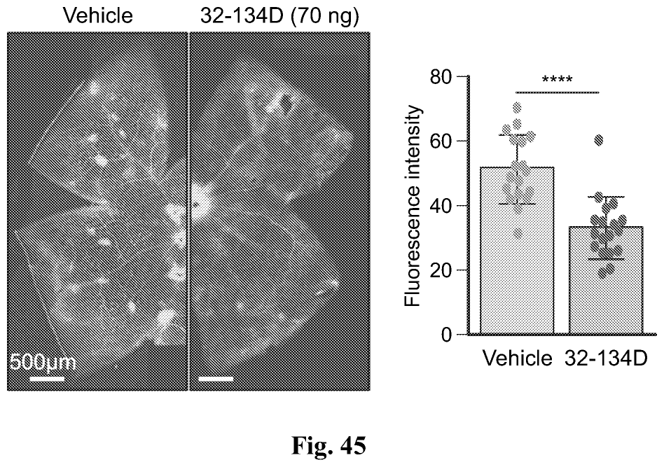

The efficacy of 32-134D following intraocular administration with 70 ng, as well as a lower dose (14 ng), in OIR mice at P12 (J. Zhang et al., J Clin Invest 131 (2021)) was determined. A marked reduction in HIF-1α and HIF-2α protein accumulation was noted at their respective peak expression at P13 and P14, respectively following a single 14-ng intraocular injection of 32-134D (J. Zhang et al., J Clin Invest 131 (2021)) (FIG. 43). This resulted in decreased expression of mRNAs encoding VEGF, its HIF-regulated endothelial cell surface receptor, VEGF Receptor 2 (KDR), as well as other key HIF-regulated angiogenic mediators in OIR mice at P17 (FIG. 44). Accordingly, treating STZ mice that were hyperglycemic for 6 months with a single intraocular injection with 70 ng 32-134D effectively inhibited vascular hyperpermeability (FIG. 45).

### Sustained Efficacy of Therapeutic Doses of 32-134D Following Intraocular Administration of Higher Doses of 32-134D in Mice.

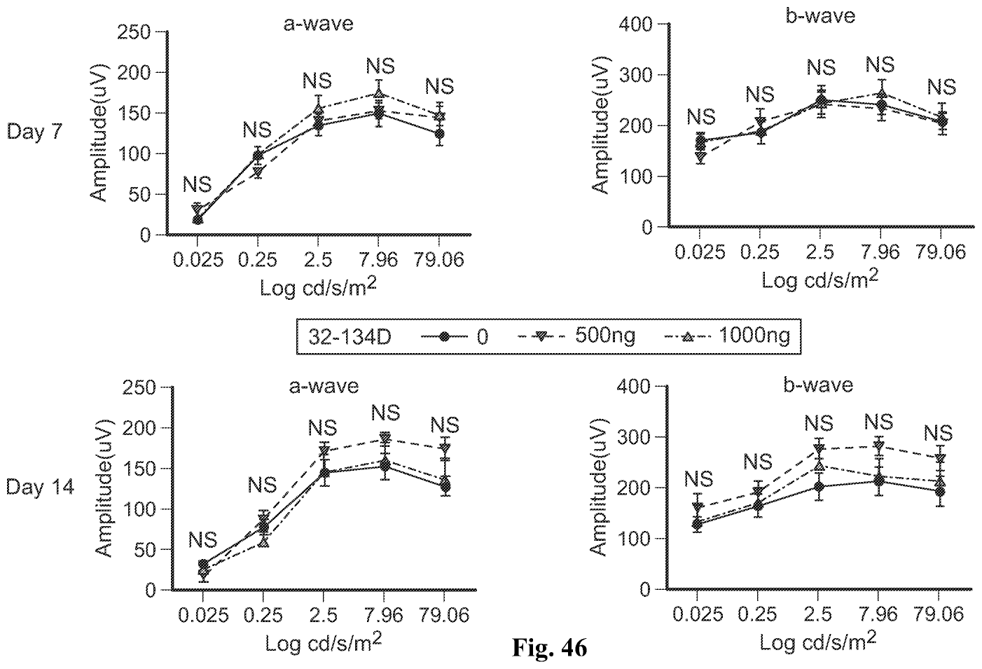

To assess whether a higher dose of 32-134D could facilitate less frequent administration, the safety of higher doses of intraocular administration of 32-134D was assessed. To this end, ERGs up to 2 weeks following a single intraocular injection of high doses (500 and 1,000 ng) of 32-134D were examined. There was no ERG evidence of retina toxicity at any of the doses of 32-134D tested (FIG. 46).

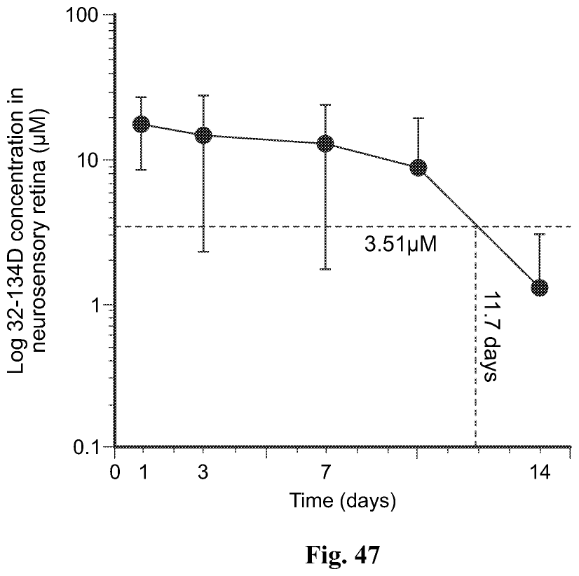

Pharmacokinetic analysis of 32-134D following intraocular administration was performed using a dose of 280 ng (a dose below the highest dose tested that was demonstrated to be safe in mice). Using LC-MS/MS to quantify the concentration of 32-134D in retina tissue over 14 days following a single intraocular injection of 280 ng, the Cₘₐₓ was 18 nmol/g in the neurosensory retina (FIG. 47), similar to the level achieved with 70 ng. However, the AUCₗₐₛₜ was 147 nmol*day/g through 14 days and the concentration of 32-134D in the neurosensory retina exceeded the in vitro IC₅₀ of 3.5 μM for 11.7 days, both equating to double the effects of the 70-ng dose (FIG. 47). The terminal half-life (T_{1/2}) was 1.8 days at 280 ng. Collectively, these data demonstrate that 32-134D is a safe and effective inhibitor of HIF activity and retinal vascular pathology, with a remarkably wide therapeutic window with the potential for sustained efficacy following a single intraocular administration.

### Example 4: NCI-60 Virtual Screen

The Pattern Comparison Analysis Tool and CellMiner Database Version 2.1 (Developmental Therapeutics Program, National Cancer Institute) were accessed at https://discover.nci.nih.gov/cellminer/.

### Example 5: Luciferase Reporter Assay

Hep3B-c1 cells (Zhang 2008), which are stably co-transfected with reporter plasmids p2.1, a hypoxia-inducible firefly luciferase reporter gene containing a hypoxia response element (HRE), and pSVR, a constitutively expressed Renilla luciferase reporter gene, were seeded on 24-well plates. Cells were treated with compounds the following day and exposed to 20% or 1% O₂ for 24 hours. For transient transfection, cells were seeded, transfected with the listed plasmids and compounds, and exposed to 20% or 1% O₂ for 24 hours. FLuc/RLuc ratios were determined using the Dual Luciferase Reporter Assay System (Promega) and the VICTOR Nivo plate reader (PerkinElmer).

### Example 6: Cell Culture

Human Hep3B and mouse Hepa1-6 cells were purchased from ATCC and grown in high-glucose (4.5 mg/ml) Dulbecco's modified Eagle's medium supplemented with 10% (vol/vol) fetal bovine serum and 1 penicillin/streptomycin at 37° C. in a 5% CO₂/95% air incubator (20% O₂). Human cell line identity was authenticated by analysis of short tandem repeats, and all cell lines were maintained mycoplasma free, using PCR-based assays conducted at the Johns Hopkins University Genetic Resources Core Facility. Cells were subjected to hypoxia in a controlled atmosphere chamber (PLAS Labs) with ambient gas mixture containing 1% O₂ and 5% CO₂.

### Example 7: RT-qPCR Assays

RNA was extracted using TRIzol Reagent (ThermoFisher). Quantitative Real-time PCR was conducted using SYBR Green PCR Master Mix (BioRad) and reactions were run using the BioRad thermal cycler (BioRad). Primer sequences are listed in Table 3. The mRNA expression of target genes was normalized to the expression of 18S rRNA and the fold change (FC) was calculated based on the threshold cycle (Ct) as

FC=2^{-Δ(ΔCt)} where ΔCt=Ct_{target gene}−Ct_{18S rRNA} and Δ(ΔCt)=ΔCtₜᵣₑₐₜₘₑₙₜ−ΔCt_{control}.

### Example 8: Immunoblot and Immunoprecipitation Assays

Whole cell lysates were prepared in RIPA buffer (50 mM Tris HCl [pH 7.5], 1 mM β-mercaptoethanol, 150 mM NaCl, 1 mM Na₃VO₄, 1 mM NaF, 1 mM EDTA, 0.25% sodium deoxycholate, and 1% Igepal CA-630) supplemented with protease inhibitors (PI). Blots were probed with antibodies against antibodies listed in Table 4.

### Example 9: Chromatin Immunoprecipitation (ChIP) Assays

Cells were seeded overnight and then exposed to 20% or 1% O₂ in the presence of compound or vehicle for 16 hours. Protein was cross-linked to DNA by addition of 37% formaldehyde to culture medium for 10 minutes at 37° C. and quenched by addition of 0.1 M glycine. Cells were washed with and collected in 5 ml of cold PBS with PI. Cells were pelleted and resuspended in SDS lysis buffer (50 mM Tris-HCl [pH 8.1], 10 mM EDTA, 1% SDS) containing PI and incubated on ice for 10 minutes. Lysates were sonicated to produce DNA fragments ranging from 200 to 900 bp and centrifuged for 10 minutes at 4° C. Supernatants were collected and diluted 10-fold with dilution buffer and precleared with 20 ml of salmon sperm DNA/protein A agarose slurry and 20 ml were reserved as input control. Antibody (2 g) was added and samples were rotated overnight at 4° C. Immune complexes were precipitated with 50 μl of salmon sperm DNA/protein A agarose slurry. Pelleted beads were washed serially using low-salt wash buffer (20 mM Tris-HCl [pH 8.1], 150 mM NaCl, 2 mM EDTA, 0.1% SDS, 1% Triton X-100); high-salt wash buffer (20 mM Tris-HCl [pH 8.1], 500 mM NaCl, 2 mM EDTA, 0.1% SDS, 1% Triton X-100), LiCl wash buffer (10 mM Tris-HCl [pH 8.1], 0.25 M LiCl, 1% Nonidet P-40, 1% deoxycholate, 1 mM EDTA), and TE buffer (10 mM Tris-HCl [pH 8.1], 1 mM EDTA). Elution buffer (1% SDS-0.1 M NaHCO₃) was added and eluates were heated at 65° C. overnight to reverse cross-linking. Eluates were treated with proteinase K for 1 hour at 45° C. and resultant DNA was purified by extraction in phenol:chloroform:isoamyl alcohol (25:24:1, v/v/v) and isopropanol precipitation. The pellet was washed with 70% ethanol and resuspended in water for qPCR analysis.

### Example 10: Animal Studies

Protocols were approved by the Johns Hopkins University Animal Care and Use Committee and used in accordance with the NIH Guide for the Care and Use of Laboratory Animals (National Research Council, 2011). Female nude mice (NCI Athymic NCr-nu/nu) and male C57L/J mice were purchased from Charles River and The Jackson Laboratory, respectively. Hep3B (8×106) and Hepa1-6 (1×107) cells were implanted subcutaneously in 6- to 8-week-old mice, and tumor volume was calculated: V=abc×0.52. For Hep3B tumor studies, once tumors reached 100-150 mm3, mice were randomized into groups to receive daily intraperitoneal injection of either vehicle or 32-134D. For Hepa1-6 tumors, mice were randomized to receive treatment once tumors reached a size of 50-100 mm3. Tumors were harvested 4 hours after the last treatment.

### Example 11: Immunohistochemistry

Tumors were fixed in 10% formalin in phosphate buffer for 24 hours and placed in PBS the following day for paraffin embedding and sectioning. Anti-CD31 immunohistochemical staining, hematoxylin and eosin counterstaining, and whole slide scanning were performed by NDB Bio (wvvwwndbbjo.m).

### Example 12: ELISA

Human (EPO, SCF, SDF1a, VEGFA) and mouse (Cxcl1, Cxcl2, Cxcl9, Cxcl10, Il6, Il10, IL22, Vegfa) protein concentrations in tumor homogenates were determined using ELISA kits (Table 4) from Novus Biologicals. Optical density was obtained at 450 nm (corrected for readings at 570 nm) using the VICTOR Nivo plate reader (PerkinElmer). Sample protein concentration was calculated by linear regression from a standard curve.

### Example 13: Cytokine mRNA Assay

Total RNA from Hepa1-6 tumors was analyzed using the RT2 Profiler PCR Array of Mouse Cytokines and Chemokines (Qiagen, catalog number 330231 PAMM-150ZA) according to the manufacturer's instructions.

### Example 14: Flow Cytometry

Hepa1-6 tumors were digested with collagenase (1 mg/ml) at 37° C. for 30 minutes and the resulting single cell suspension was passed through a 70-μm cell strainer and washed twice with cold PBS. Cells were resuspended in FC buffer for subsequent flow cytometry analysis. Cells were stained with the following antibodies (see Table 4) to capture different immune cell populations: TANs: AF405-conjugated anti-CD11b, FITC-conjugated anti-Ly6C and APC-conjugated anti-Ly6G; TADCs: AF-405-conjugated anti-CD11b, FITC-conjugated anti-CD11c and APC-conjugated anti-F4/80; MDSCs: AF405 conjugated anti-CD11b and FITC-conjugated anti-Ly6C; TAMs: AF405-conjugated anti-CD11b and APC-conjugated anti-F4/80; M1-TAMs: AF405-conjugated anti-CD11b, APC-conjugated anti-F4/80 and FITC-conjugated anti-CD80; M2-TAMs: AF405-conjugated anti-CD11b, APC-conjugated anti-F4/80 and FITC-conjugated anti-CD206; NK cells: FITC-conjugated anti-CD3 and APC-conjugated anti-NK1.1; cytotoxic NK cells: FITC-conjugated anti-CD3, APC-conjugated anti-NK1.1 and AF405-conjugated anti-CD314; effector T cells: PE-conjugated anti-CD8A and AF405-conjugated anti-IFN-g; activated T cells: PE-conjugated anti-CD8A, FITC-conjugated anti-CD69, and APC conjugated anti-CD44; regulatory T cells: APC-conjugated anti-CD4, FITC-conjugated anti-CD25, and PE-conjugated anti-FoxP3; CD8A T cells: AF405-conjugated anti-CD45 and PEconjugated anti-CD8A; and CD4 T cells: AF405-conjugated anti-CD45 and APC-conjugated anti-CD4. Live cells were gated using the side-scatter and forward-scatter plots and data were acquired using the FACSDiva software (Becton Dickinson). Cell populations were gated using the unstained control and single stained cell samples. Data analysis was performed using FlowJo software.

### Example 15: Statistical Analysis

Data were expressed as mean±SEM. Differences were considered statistically significant for p<0.05. Data were analyzed using the 2-tailed Mann-Whitney nonparametric t test for comparisons between two groups or ANOVA with the Tukey-Kramer test for multiple comparisons. Analyses of association were performed using the Spearman rank correlation test. Kaplan-Meier survival analysis was performed using the log rank test through an online tool (kmplot.com), using the median mRNA expression level for stratification and overall survival at 3 years as the outcome measure for hazard ratio calculation.

### Example 16: Safety Studies

### Safety Profile of 32-134D in Mice.

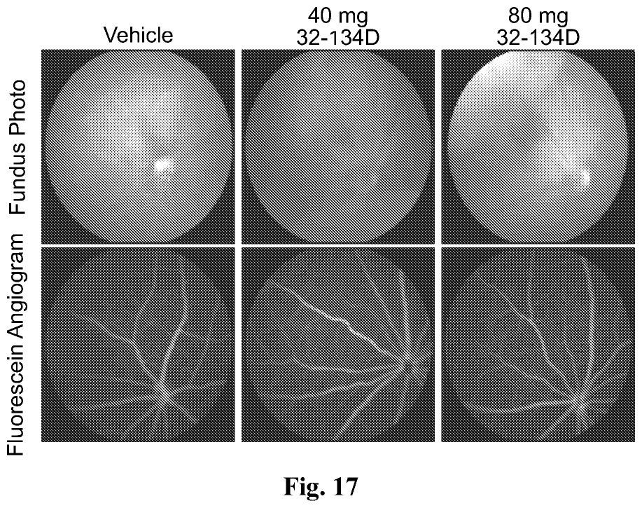

Fundus photos and fluorescein angiographic images of the retina of C57BL/6 mice on day 30 following 5 daily intraperitoneal (IP) injections (day 0 to day 5) with 32-134D at 40 or 80 mg/kg compared to vehicle control (FIG. 17). Photos and angiographic images demonstrate intact retinal vasculature and absence of capillary leakage in all animals tested.

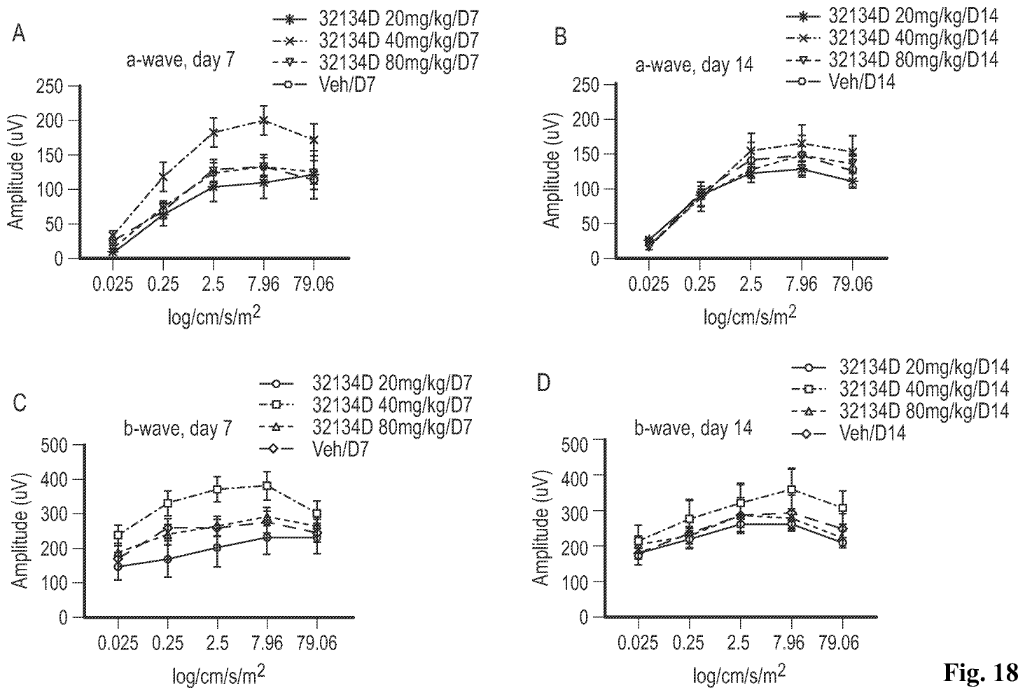

ERG measurements taken in C57BL/6 mice at day 7 (D7) and day 14 (D14) following 5 daily IP injections (day 0 to day 5) with vehicle control (Veh) or 32-134D at 20, 40, or 80 mg/kg/day. A-wave compared at different doses for 32-134D at D7 (FIG. 18A) and D14 (FIG. 18B) demonstrating no statistically significant difference between 32-134D and vehicle control. Comparison of b-waves at different doses for 32-134D at D7 (FIG. 18C) and D14 (FIG. 18D) demonstrating no statistically significant difference between 32-134D and vehicle control.

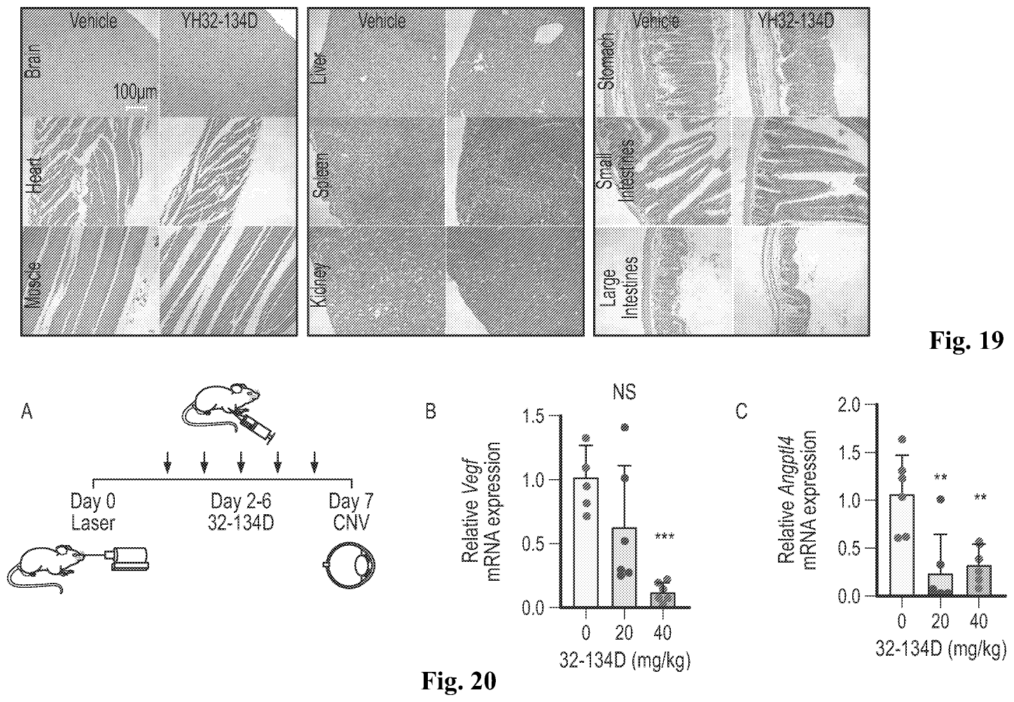

To survey for possible organ damage following systemic administration, three-month-old mice were treated with the 80 mg/kg/day of 32-134D by IP injection for five consecutive days (days 0-4). On day 30, mice were sacrificed, perfused with PBS, and tissues were harvested and fixed in 4% paraformaldehyde (PFA). Harvested tissues included the brain, heart, muscle, liver, spleen, kidney, stomach, small intestine, and large intestine. Tissues were embedded with paraffin, sectioned at 10 m, and stained with hematoxylin and eosin. Images were taken at 20× magnification. There was no evidence of toxicity in any of the tissues examined (FIG. 19). Similar results were obtained for animals treated with 20 or 40 mg/kg/day.

### Example 17: Efficacy Studies

### Efficacy of 32-134D in Mice.

The laser CNV model for neovascular (wet) age-related macular degeneration was used to assess the efficacy of 32-134D in blocking HIF-regulated gene expression in the eye (FIG. 20A). C57BL/6 mice were treated with laser on day 0. Beginning on day 2, mice were treated with 5 daily intraperitoneal (IP) injections (day 2 to day 6) with 32-134D at 40 mg/kg or with vehicle control. On day 7, choroidal flat mounts were prepared and stained with isolectin-B4 to examine the CNV lesions. The expression of Vegf (FIG. 20B) and Angpt14 (FIG. 20C) mRNA was also measured at day 7 following laser treatment. Vegf mRNA was significantly decreased in eyes from mice treated with 32-134D at the dose of 40 mg/kg. Angpt14 mRNA expression significantly decreased in eyes from mice treated with 20 or 40 mg/kg of 32-134D.

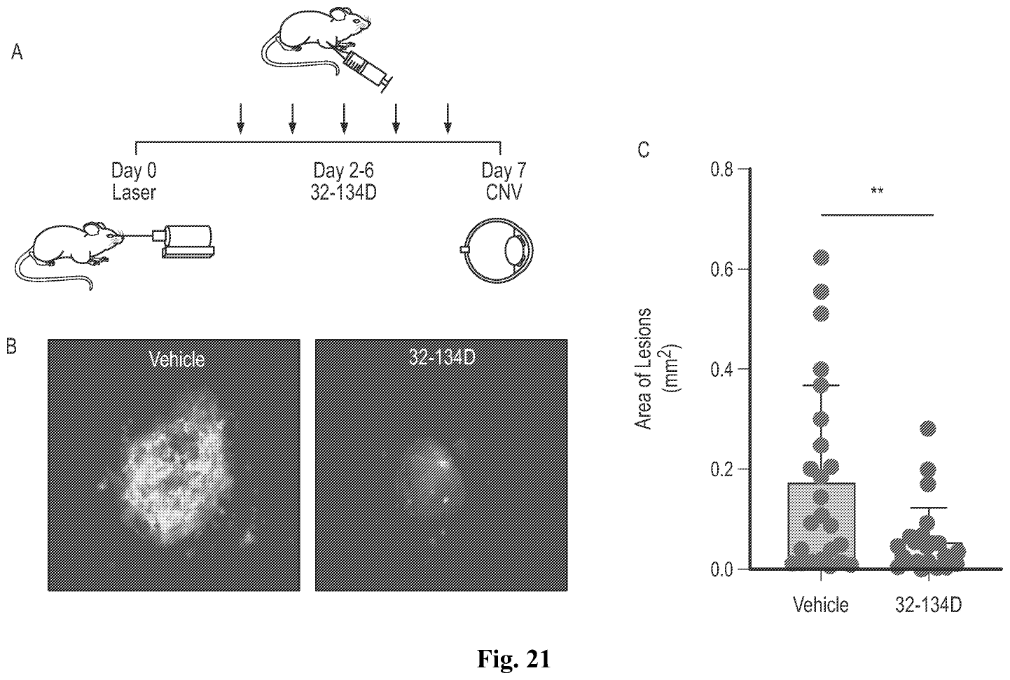

Results of the laser CNV model of neovascular (wet) age-related macular degeneration are shown in FIG. 21A. C57BL/6 mice were treated with laser on day 0. Beginning on day 2, mice were treated with 5 daily IP injections (day 2 to day 6) with 32-134D at 40 mg/kg or with vehicle control. On day 7, mice were euthanized and choroidal flat mounts were stained with isolectin-B4 to examine the area of CNV lesions. Images of representative lesions at high magnification (20×) demonstrate a marked decrease in CNV in mice treated with 32-134D compared to vehicle control (FIGS. 21B-C).

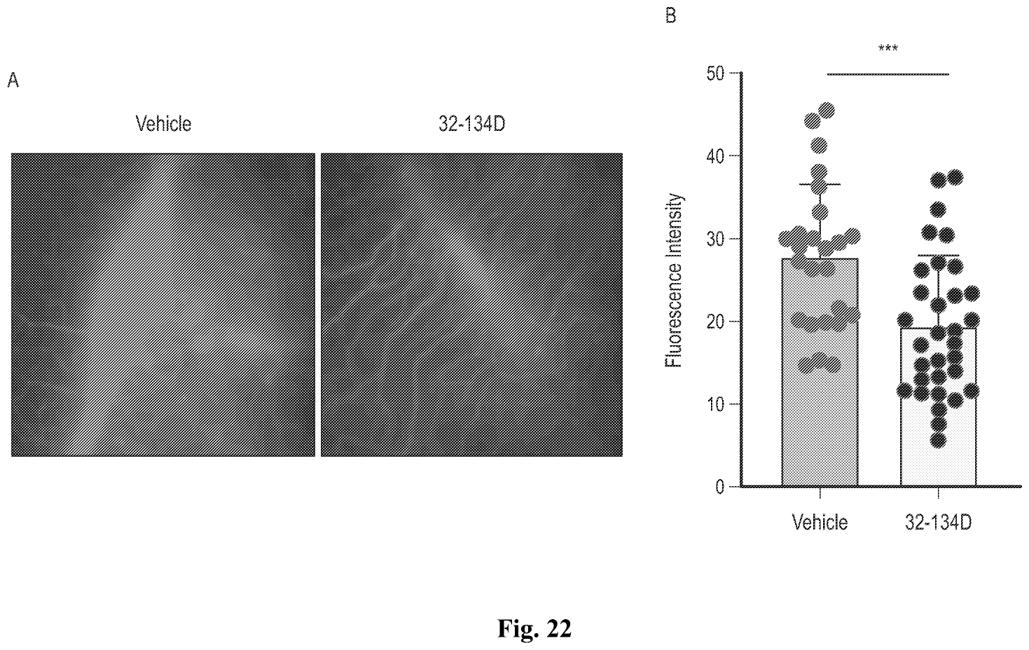

The ability of 32-143D to inhibit vascular permeability in diabetic patients was evaluated using the streptozotocin (STZ) model for diabetic macular edema. 8-to-10-week-old C57BL/6 mice were administered STZ by IP injection. Hyperglycemia (serum glucose >250 mg/dL) was confirmed by glucometer 2 weeks later. After 6 months of sustained hyperglycemia, mice were treated with 5 daily IP injections with 32-134D at 40 mg/kg or with vehicle control. 24 hours after the fifth IP injection, mice were euthanized, Evans blue perfusion was performed, and retina flat mounts were examined for vascular leakage. Images of representative vascular leakage at high magnification (20×) for animals treated with vehicle vs 32-134D demonstrates reduced leakage following treatment with 32-134D (FIG. 22A). Quantitation of vascular permeability by relative fluorescence intensity of Evans blue dye demonstrated significantly decreased vascular leakage in 32-134D treated mice compared to vehicle control (FIG. 22B).

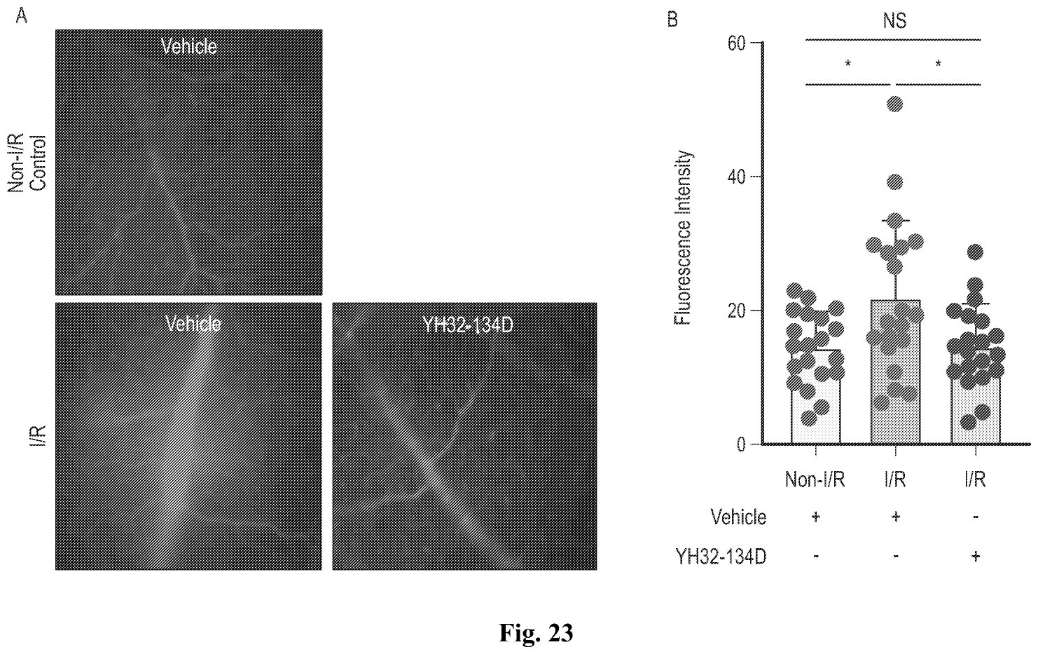

The ability of 32-134D to inhibit vascular permeability in retinal vein occlusion patients was evaluated using the ischemia/reperfusion (I/R) model for vascular permeability in ischemic retinal disease. Transient ischemia was induced in the left eye in 8-to-10-week-old male C57BL/6 mice; the right eye was used as the non-I/R control. Following ischemia, mice were treated with 5 daily IP injections of vehicle control or 32-134D at 40 mg/kg. 24 hours after the fifth IP injection, mice were euthanized, Evans blue perfusion was performed, and retina flat mounts were examined for vascular leakage. Images of representative vascular leakage at high magnification (20×) for animals treated with vehicle vs 32-134D demonstrate a reduction in leakage following treatment with 32-134D (FIG. 23A). Quantitation of vascular permeability by relative fluorescence intensity of Evans blue dye demonstrated a significant increase in vascular leakage in I/R eyes compared to non-I/R eyes, and a significant reduction in vascular leakage in 32-134D treated I/R eyes compared to vehicle control (FIG. 23B). There was no difference between non-/R (healthy) eyes and I/R eyes treated with 32-134D.

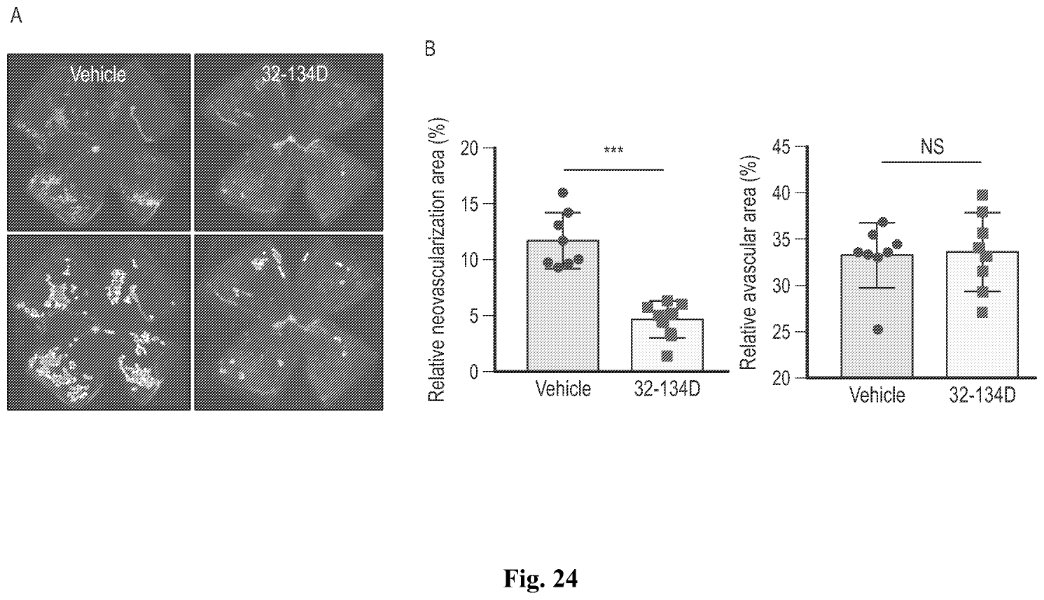

The ability of 32-143D to inhibit ischemia-induced retinal neovascularization was evaluated using the oxygen-induce retinopathy (OIR) model for retinal neovascularization in ischemic retinal diseases, such as retinopathy of prematurity, proliferative diabetic retinopathy, ischemic retinal vein occlusions, or sickle cell retinopathy. Postnatal day 7 (P7) newborn C57BL/6 pups were exposed to hyperoxia (75% O₂) for 5 consecutive days before being returned to room air at P12. Beginning at P13, pups were treated with 4 daily IP injections with 32-134D at 20 mg/kg or with vehicle control. 24 hours after the fifth IP injection, at P17, retinal flat mounts were stained with isolectin-B4 to examine the development of retinal neovascularization. Images of representative retinal flat mounts at low magnification (10×) for pups treated with vehicle vs 32-134D demonstrated a decrease in retinal neovascular lesions in mice treated with 32-134D compared to vehicle (FIG. 24A). Quantitation of retinal neovascularization (FIG. 24B; left) and avascular retina (FIG. 24B; right) demonstrated a significant decrease in neovascularization in 32-134D treated OIR eyes compared to vehicle control. The area of avascular retina in OIR eyes treated with 32-134D was not significantly different from vehicle control.

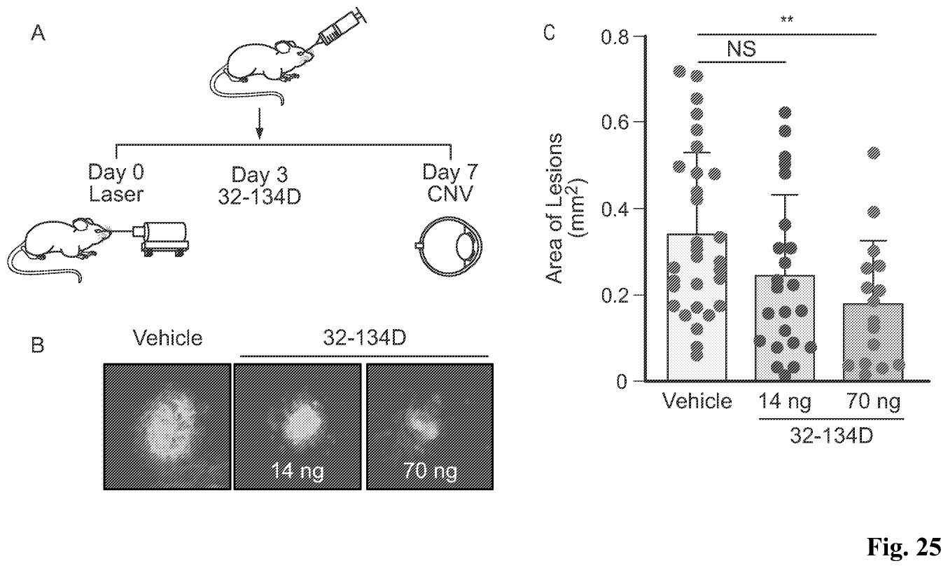

To evaluate the efficacy of 32-134D following intraocular administration, the laser CNV model for neovascular (wet) age-related macular degeneration was used (FIG. 25A). C57BL/6 mice were treated with laser on day 0. On day 3, mice were treated with a single IVT injection with 32-134D (1 μl of 14 or 70 ng/μl solution) or with vehicle control. On day 7, mice were euthanized and choroidal flat mounts were stained with isolectin B4 to examine the area of CNV. Images of representative choroidal neovascular lesions at high magnification (20×) for animals treated with vehicle vs 32-134D demonstrated decreased CNV in mice treated with 32-134D compared to vehicle control (FIG. 25B). Quantitation of CNV area demonstrated a significant decrease in mice treated with 70 ng of 32-134D compared to vehicle control (FIG. 25C).

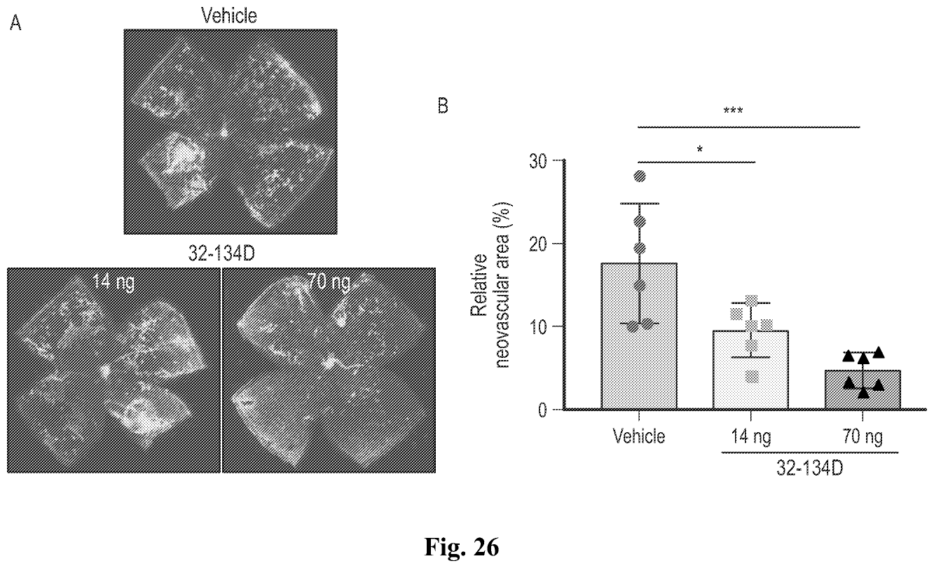

The efficacy of 32-134D following IVT administration was also evaluated in the OIR model. At P14, pups were treated with a single intravitreal injection with 32-134D (14 or 70 ng) in 1 ul or with vehicle (DMSO) control. At P17, retinal flat mounts were stained with isolectin-B4 to examine the development of retinal neovascularization. Images of representative retinal flat mounts at low magnification (10×) for pups treated with DMSO vehicle vs 32-134D demonstrate a reduction in neovascular lesions in mice treated with 32-134D compared to DMSO control (FIG. 26A). Quantitation of retinal neovascularization demonstrated a significant decrease in 32-134D treated OIR eyes compared to vehicle control (FIG. 26B).

The efficacy of 32-134D following IVT administration was also evaluated in the STZ model. STZ mice that were hyperglycemic for 6 months were treated with a single intraocular injection with 70 ng 32-134D in 1 ul or with vehicle (DMSO) control. 5 days later vascular hyperpermeability was assessed by examining leakage of intravascular Evans blue dye in STZ mice. Images of representative retinal flat mounts at low magnification (10×) for pups treated with vehicle (left) vs 32-134D demonstrate a reduction in vascular leakage in mice treated with 32-134D compared to vehicle control (FIG. 45, left). Quantitation of vascular leakage demonstrated a significant decrease in 32-134D treated STZ eyes compared to vehicle control (FIG. 45, right).

### Materials and Methods

Cells culture and reagents: MIO-M1 cells were a gift from Dr. Astrid Limb (University College London, Institute of Ophthalmology, London, United Kingdom). MIO-M1 and HUVEC cells were cultured in DMEM with 10% (vol/vol) FBS (Quality Biological) and 1% penicillin/streptomycin (Cellgro). Hypoxia chambers were used to expose cells. Evans Blue (E2129) was purchased from Sigma-Aldrich.

Mice: Eight-week-old pathogen-free female C57BL/6 mice were obtained from Jackson Laboratory. Timed pregnant C57BL/6 mice were obtained from Charles River Laboratories. All animals were treated in accordance with the Association for Research in Vision and Ophthalmology Statement for the Use of Animals in Ophthalmic and Vision Research and the guidelines of the Johns Hopkins University Animal Care and Use Committee.

Retinal organoids: An hiPSC line derived from CD34+ cord blood was used in this study (A18945, ThermoFisher Scientific) (P. W. Burridge et al., 2011, PLoS One 6, e18293). Undifferentiated hiPSCs and derived retinal organoids were routinely tested for Mycoplasma contamination by PCR. Cell culture, retinal differentiation, and organoid formation were conducted as previously described (X. Zhong et al., 2014, Nat Commun 5, 4047). Retinal organoids at 120 days of differentiation were used for experiments.

Immunoblot assays and ELISAs: Cell, neurosensory retina, and D120 retinal organoid lysates were subjected to 4-15% gradient SDS/PAGE (Invitrogen). Immunoblot assays were performed as previously described (X. Xin et al., 2013, Proc Natl Acad Sci USA 110, E3425-3434).

Vascular permeability by Evans blue tracer method: Mice were anesthetized with a mixture of ketamine (50 mg/kg) and xylazine (5 mg/kg) and subjected to Evans blue (Sigma) (50 mg/kg) perfusion by penile vein injection. After 10 minutes, animals were sacrificed by CO₂ asphyxiation. The eyes were enucleated and immediately immersed in 4% (wt/vol) paraformaldehyde for 2 hours at room temperature. Retinas were isolated and whole mounts prepared on clean glass slides and mounted vitreous side up under coverslips with antifade medium (Vectashield; Vector Laboratories, Burlingame, CA). Images were captured by Zeiss fluorescent microscope or confocal microscopy and fluorescence quantified by image J software (NIH). Retinas from diabetic mice demonstrate prominent leakage for from major and branched capillaries compared to control. Blinded quantification of the number of leaking vessels was performed by manual counting major leakage from capillaries from retinas of each group. Non-capillary artifactual Evans blue or FITC-dextran leakage due to flat mount processing as well as minor leakages were not included in the quantitation. Quantification of fluorescence intensity was performed by ImageJ software by selecting leakages from each petal of retina when leakage was present; in the absence of leakage, random images were taken of each petal of retina. Random fields were chosen from retinas where leakages were absent to determine baseline fluorescence that arise from capillaries. For quantification, high magnifications (20×) images were taken. Whole flat mount retinas included in figures were taken using low magnification (5×).

Intraocular injections: Intravitreal injections were performed with a PLI-100A Pico-liter Microinjector (Warner Instruments, Harvard Bioscience) using pulled-glass micropipettes. Each micropipette was calibrated to deliver a 1 μl volume on depression of a foot switch. The mice were anesthetized with a ketamine (100 mg/kg) and xylazine (5 mg/kg) mixture, and under a dissecting microscope, the sharpened tip of the micropipette was passed through the sclera just posterior to the limbus into the vitreous cavity and the foot switch was depressed, which caused fluid to penetrate into the vitreous space. Test compound was injected into the vitreous cavity in OIR and STZ mice for vascular permeability test, retinal vasculature and lysates for WB and qPCR analysis.

OIR mouse model: OIR experiments were performed as previously described (M. Rodrigues et al., 2013, Diabetes 62, 3863-3873). Briefly, C57BL/6 mice were placed in 75% O₂ on P7. On P12, the mice were returned to room air and administered either by IP injection (digoxin and test compound) or by intravitreal injection. Mice with body weight less than 6 g at P17 were excluded from analysis. The data were collected from both males and females and the results combined, as there was no apparent difference between sexes. Representative images for selected time points from a minimum of three independent experiments are shown. Data from 3-8 pups were obtained at each time point.

STZ-induced diabetes mouse model: 8-to-10 week-old male mice received an IP injection of STZ at 40 mg/kg of body weight in 0.1 M of citrate buffer (pH 4.5) for 5 consecutive days. Normal chow and 10% sucrose water were provided during this period. 10% sucrose was discontinued and replaced with regular water on experimental day 6. Blood glucose was measured on experimental day 28 after 16 hours fasting and mice with blood glucose level >250 mg/dL were considered diabetic.

Immunofluorescence assays: Details for antibodies are provided in Table 5. Immunofluorescence assays in retinal organoids and mouse retinal tissues were performed as previously described (J. Zhang et al., 2021, J Clin Invest 131). Briefly, mice were sacrificed using CO₂ asphyxiation, eyes of treated mice were enucleated and fixed with a solution of 4% paraformaldehyde in PBS (Thermo Scientific™) for two hours at RT, followed by washing with PBS for 10 minutes in a shaker. Retinas were then isolated and incubated in 0.5% BSA solution overnight at 4° C. Retinas were then washed with PBS 3 times for 10 minutes in the shaker and then stained with isolectin B4 (Invitrogen; 1:200 dilution in PBS) overnight at 4° C. After washing 3 times for 10 minutes each in the shaker, the retinas were mounted. Images were captured with Zeiss fluorescent microscope. The images were selected (8 fields per eye, 2 fields per petal, including area of neovascularization) and fluorescence intensity was measured by ImageJ software (NIH).

Reverse transcription and quantitative real-time PCR: Total RNA was isolated from cultured cells or retinas with PureLink™ RNA Mini Kit (Invitrogen #12183025), and cDNA was prepared with MuLV Reverse Transcriptase (Applied Biosystems). Quantitative real-time PCR was performed with Power SYBR Green PCR Master Mix (Applied Biosystems) and MyiQ Real-Time PCR Detection System (Bio-Rad). Normalization was done using cyclophilin A for mouse tissue and human cell lines. Primers are listed in Table 6.

Flash scotopic ERGs: All procedures were performed under dim red light. Mice were dark adapted overnight, anesthetized with ketamine (50 mg/kg) and xylazine (5 mg/kg) and pupils were dilated with a topical drop of tropicamide (1%). Scotopic ERG responses were measured using the Celeris ERG stimulator (Diagnosys, Lowell, MA) at flash intensities of 0.025, 0.25, 2.5, 7.96, and 79.06 cd/s/m2. ERGs were measured simultaneously from both eyes with a ground electrode placed into the forehead between the eyes and a reference electrode into the hip.

Fluorescein angiography in mice: To evaluate the effect of 32-134D on the retinal vasculature and vascular permeability, experimental animals were treated with 5 consecutive daily IP injections with 32-134D at 40 or 80 mg/kg. On day 30, mice were anesthetized using intramuscular injection with ketamine (50 mg/kg) and xylazine (5 mg/kg). Pupils were then dilated using 1% tropicamide eye drops and mice were placed on the imaging platform of the Phoenix Micron III retinal imaging microscope (Phoenix Research Laboratories, Pleasanton, CA). Goniovisc 2.5% (hypromellose; Sigma Pharmaceuticals, LLC, Monticello, IA) was applied liberally to keep the eye moist during imaging studies. Fundus photos were taken before administering 10 to 20 μl of 10% fluorescein sodium (Apollo Ophthalmics, Newport Beach, CA) IP injection. Rapid acquisition of fluorescein angiographic images was then performed for 5 minutes. Fluorescein leakage manifests as indistinct vascular borders progressing to diffusely hazy fluorescence. Fluorescein leakage was compared between different groups by quantifying the fluorescence intensities collected after 1, 2, and 3 minutes following fluorescein injection using ImageJ software (National Institutes of Health, Bethesda, MD).

Mouse pharmacokinetics: C57BL/6 mice were administered 32-134D at 70 ng or 280 ng as a single intraocular injection. Mice (at least 3 mice per time point) were euthanized at 1, 3, 7, or 14 days after injection. For the 280-ng dose, a 10-day time point was added. 32-134D was quantified in retina by LC-MS/MS as previously described (S. Salman et al., 2022, J Clin Invest 132) with the following modifications. Retina tissue samples were homogenized in 200 L of 1×PBS (pH 7.4) before extraction. The standard curve and quality control samples were prepared in 1×PBS as a surrogate matrix. Retina tissue samples were then quantitated in nmol/g as: nominal concentration (nM)×initial dilution ([tissue weight (mg)+volume of solvent (μl)]/tissue weight (mg)). For all samples <10 nM, the value was reported as below the limits of detection. If at least on samples was >10 nM and one was <10 nM, then one half that value (5 nM) was imputed to calculate the concentration for that specimen and utilized in the average and standard deviation calculations.

Pharmacokinetic parameters: These were calculated from mean concentration-time data using noncompartmental methods in Phoenix WinNonlin version 8.3 (Certara, Princeton, NJ, USA). The Cmax and time to Cmax (Tmax) were the observed values. The AUCₗₐₛₜ was calculated using the log-linear trapezoidal method to the last quantifiable time point. The λz was determined from at least 3 points on the slope of the terminal phase of the concentration-time profile using a 1/y2 weighting factor. The T1/2 was determined by dividing 0.693 by az. T1/2 was not reported if the correlation coefficient (r2) for λz was less than 0.9. The total time above the IC₅₀ (3.51 nmol/g) was calculated.

Bioanalytical method for mouse pharmacokinetics: Retina tissue samples were homogenized in 200 μL of 1×PBS (pH 7.4) before extraction. The standard curve and quality control samples were prepared in 1×PBS as a surrogate matrix for all matrices. Tissue homogenate or PBS (25 μL) was added to a borosilicate glass test tube and mixed with 150 μL of acetonitrile containing the internal standard (1 ng/mL of EXP-3179). For blank samples, 150 μL of acetonitrile was added without internal standard. Samples were vortex-mixed and centrifuged (1200×g for 5 minutes at ambient temperature) and transferred to an autosampler vial. Then 2 μL was injected onto the liquid chromatography system using a temperature-controlled autosampling device operating at approximately 10° C. Chromatographic analysis was performed using a Waters Acquity™ Ultra Performance LC. Separation of the analyte from potentially interfering material was achieved at ambient temperature using Halo C18 column (50×2.1 mm i.d.) with a 2.7-μm particle size. The mobile phase used for the chromatographic separation was composed of 0.1% (v/v) formic acid in water (mobile phase A) and 0.1% (v/v) formic acid in acetonitrile (mobile phase B) with a flow rate of 0.4 mL/minute. The initial mobile phase composition was 60% mobile phase A and 40% mobile phase B. From 0.5 to 2.0 minutes, mobile phase B was increased linearly from 40% to 100% and maintained until 3.0 minutes. From 3.0 to 3.1 min, the gradient decreased to 40% mobile phase B and the conditions were maintained until 5 minutes to re-equilibrate the column for the next injection. The column effluent was monitored using an AB Sciex Triple Quadrupole 5500 mass spectrometer. The instrument was equipped with an electrospray interface, operated in a positive mode and controlled by the Analyst v1.7 software. The settings were as follows: curtain gas 20 psi, medium collision gas, ion spray voltage 5500 V, probe temperature 450° C., ion source gas one 30 psi, ion source gas two 40 psi, and entrance potential 10. The collision cell exit potentials were 14.0 and 6.0 for 32-134D and the internal standard, respectively. The declustering potential was 141 and 80 for 32-134D and the internal standard, respectively. The collision energies were 43 and 25 for 32-134D and internal standard, respectively. MRM m/z transitions were the following: 474.6, 393.8 and 421.0, 207.1 for 32-134D and the internal standard, respectively. Dwell time was 150 milliseconds. The calibration curve for 32-134D was constructed from the peak area ratio of the analyte to the peak area of its internal standard using the least-squares quadratic regression analysis with 1/×2 weight over the range of 10-2,110 nM with dilutions of up to 1:10 (v/v).

Statistical analysis: In all cases, results are shown as a mean value SD or mean value±SEM from at least three independent experiments. Statistical analysis was performed with Microsoft Excel and Prism 8.0 software (GraphPad). Statistical differences between two or multiple heterogenous groups were determined by unpaired Student's t-test and one-way or two-way ANOVA. Analysis of this data was performed in MATLAB and Excel. *P<0.05; **P<0.01; ***P<0.001; ****P<0.0001. NS=not significant.

TABLE 1
Expression of cytokine mRNAs in tumors from mice
treated with 32-134D (test group) or vehicle (control group)
as determined by an RT-qPCR array.

| | | | | | Fold Up- or | | |
|---|---|---|---|---|---|---|---|
| | | | Fold Change | | Down-Regulation | Y axis | X axis |
| | | | Test Group/ | T-TEST | Test Group/ | (−Log10 | (Log2(fold |
| | Symbol | Well | Control Group | p value | Control Group | (pValue)) | change) |
| 1 | Adipoq | A01 | 0.43 | 0.0159 | −2.31 | 1.8 | −1.21 |
| 2 | Bmp2 | A02 | 0.51 | 0.1529 | −1.95 | 0.8 | −0.96 |
| 3 | Bmp4 | A03 | 0.64 | 0.3124 | −1.96 | 0.5 | −0.64 |
| 4 | Bmp6 | A04 | 0.44 | 0.0138 | −2.27 | 1.9 | −1.18 |
| 5 | Bmp7 | A05 | 0.70 | 0.4516 | −1.43 | 0.3 | −0.51 |
| 6 | Ccl1 | A06 | 0.63 | 0.4314 | −1.58 | 0.4 | −0.66 |
| 7 | Ccl11 | A07 | 1.03 | 0.7782 | 1.03 | 0.1 | 0.04 |
| 8 | Ccl12 | A08 | 4.11 | 0.0251 | 4.11 | 1.6 | 2.04 |
| 9 | Ccl17 | A09 | 0.68 | 0.4482 | −1.47 | 0.3 | −0.56 |
| 10 | Ccl19 | A10 | 0.54 | 0.0011 | −1.86 | 3.0 | −0.89 |
| 11 | Ccl2 | A11 | 1.35 | 0.2081 | 1.35 | 0.7 | 0.44 |
| 12 | Cc120 | A12 | 0.42 | 0.0056 | −2.41 | 2.3 | −1.27 |
| 13 | Ccl22 | B01 | 0.31 | 0.0027 | −3.18 | 2.6 | −1.67 |
| 14 | Ccl24 | B02 | 1.21 | 0.4075 | 1.21 | 0.4 | 0.27 |
| 15 | Ccl3 | B03 | 1.06 | 0.7500 | 1.06 | 0.1 | 0.09 |
| 16 | Ccl4 | B04 | 0.72 | 0.3408 | −1.39 | 0.5 | −0.47 |
| 17 | Cc15 | B05 | 0.92 | 0.5575 | −1.09 | 0.3 | −0.13 |
| 18 | Ccl7 | B06 | 1.03 | 0.7626 | 1.03 | 0.1 | 0.04 |
| 19 | Cd40lg | B07 | 0.52 | 0.0452 | −1.91 | 1.3 | −0.93 |
| 20 | Cd70 | B08 | 0.24 | 0.0010 | −4.19 | 3.0 | −2.07 |
| 21 | Cntf | B09 | 0.38 | 0.0275 | −2.61 | 1.6 | −1.39 |
| 22 | Csf1 | B10 | 1.02 | 0.8491 | 1.02 | 0.1 | 0.03 |
| 23 | Csf2 | B11 | 0.38 | 0.0024 | −2.64 | 2.6 | −1.40 |
| 24 | Csf3 | B12 | 0.50 | 0.0033 | −1.99 | 2.5 | −0.99 |
| 25 | Ctf1 | C01 | 0.19 | 0.0005 | −5.18 | 3.3 | −2.37 |
| 26 | Cx3cl1 | C02 | 0.53 | 0.0005 | −1.89 | 3.3 | −0.92 |
| 27 | Cxcl1 | C03 | 0.67 | 0.0072 | 1.49 | 2.1 | −0.57 |
| 28 | Cxcl10 | C04 | 1.44 | 0.0484 | 1.44 | 1.3 | 0.53 |
| 29 | Cxcl11 | C05 | 0.45 | 0.0055 | −2.24 | 2.3 | −1.16 |
| 30 | Cxcl12 | C06 | 2.26 | 0.1827 | 2.26 | 0.7 | 1.17 |
| 31 | Cxcl13 | C07 | 0.39 | 0.0003 | −2.57 | 3.5 | −1.36 |
| 32 | Cxcl16 | C08 | 0.61 | 0.0540 | −1.65 | 1.3 | −0.72 |
| 33 | Cxcl3 | C09 | 0.87 | 0.1486 | −1.14 | 0.8 | −0.19 |
| 34 | Cxcl5 | C10 | 1.05 | 0.7156 | 1.05 | 0.1 | 0.07 |
| 35 | Cxcl9 | C11 | 1.64 | 0.0001 | 1.64 | 4.0 | 0.71 |
| 36 | Fasl | C12 | 0.59 | 0.0019 | −1.70 | 2.7 | −0.77 |
| 37 | Gpi1 | D01 | 0.64 | 0.0398 | −1.57 | 1.4 | −0.65 |
| 38 | Hc | D02 | 1.77 | 0.0180 | 1.77 | 1.7 | 0.82 |
| 39 | lfna2 | D03 | 0.26 | 0.0011 | −3.85 | 3.0 | −1.95 |
| 40 | lfng | D04 | 1.63 | 0.0015 | 1.63 | 2.8 | 0.70 |
| 41 | ll10 | D05 | 0.23 | 0.0012 | −4.42 | 2.9 | −2.14 |
| 42 | ll11 | D06 | 0.24 | 0.0043 | −4.12 | 2.4 | −2.04 |
| 43 | ll12a | D07 | 0.48 | 0.0138 | −2.07 | 1.9 | −1.05 |
| 44 | ll12b | D08 | 0.29 | 0.0029 | −3.51 | 2.5 | −1.81 |
| 45 | ll13 | D09 | 0.32 | 0.0071 | −3.14 | 2.1 | −1.65 |
| 46 | ll15 | D10 | 0.84 | 0.3524 | 1.19 | 0.5 | −0.25 |
| 47 | ll16 | D11 | 0.53 | 0.0145 | −1.90 | 1.8 | −0.93 |
| 48 | ll17a | D12 | 0.33 | 0.0045 | −3.00 | 2.3 | −1.59 |
| 49 | ll17f | E01 | 0.75 | 0.0926 | −1.33 | 1.0 | −0.41 |
| 50 | ll18 | E02 | 0.89 | 0.4793 | −1.13 | 0.3 | −0.17 |
| 51 | ll1a | E03 | 0.68 | 0.4472 | −1.47 | 0.3 | −0.56 |
| 52 | ll1b | E04 | 0.34 | 0.2747 | −2.92 | 0.6 | −1.55 |
| 53 | ll1rn | E05 | 0.68 | 0.0957 | −1.47 | 1.0 | −0.56 |
| 54 | ll2 | E06 | 0.30 | 0.0190 | −3.37 | 1.7 | −1.75 |
| 55 | ll21 | E07 | 0.29 | 0.0098 | −3.46 | 2.0 | −1.79 |
| 56 | ll22 | E08 | 0.37 | 0.0042 | −2.67 | 2.4 | −1.42 |
| 57 | ll23a | E09 | 0.44 | 0.1786 | −2.25 | 0.7 | −1.17 |
| 58 | ll24 | E10 | 0.43 | 0.0232 | −2.34 | 1.6 | −1.23 |
| 59 | ll27 | E11 | 0.46 | 0.1550 | −2.19 | 0.8 | −1.13 |
| 60 | ll3 | E12 | 0.36 | 0.0041 | −2.77 | 2.4 | −1.47 |
| 61 | ll4 | F01 | 0.34 | 0.0010 | −2.91 | 3.0 | −1.54 |
| 62 | ll5 | F02 | 0.42 | 0.0710 | −2.39 | 1.1 | −1.26 |
| 63 | ll6 | F03 | 0.34 | 0.0209 | −2.95 | 1.7 | −1.56 |
| 64 | ll7 | F04 | 0.97 | 0.9540 | −1.04 | 0.0 | −0.05 |
| 65 | ll9 | F05 | 0.27 | 0.0015 | −3.69 | 2.8 | −1.88 |
| 66 | Lif | F06 | 0.40 | 0.0807 | −2.48 | 1.1 | −1.31 |
| 67 | Lta | F07 | 0.34 | 0.0331 | −2.96 | 1.5 | −1.57 |
| 68 | Ltb | F08 | 0.63 | 0.0784 | −1.58 | 1.1 | −0.66 |
| 69 | Mif | F09 | 1.12 | 0.5514 | 1.12 | 0.3 | 0.17 |
| 70 | Mstn | F10 | 0.21 | 0.0941 | −4.85 | 1.0 | −2.28 |
| 71 | Nodal | F11 | 0.29 | 0.0009 | −3.43 | 3.1 | −1.78 |
| 72 | Osm | F12 | 0.38 | 0.0329 | −2.65 | 1.5 | −1.40 |
| 73 | Pf4 | G01 | 0.68 | 0.4216 | −1.47 | 0.4 | −0.56 |
| 74 | Ppbp | G02 | 0.64 | 0.1634 | −1.56 | 0.8 | −0.65 |
| 75 | Spp1 | G03 | 1.71 | 0.0649 | 1.71 | 1.2 | 0.77 |
| 76 | Tgfb2 | G04 | 0.74 | 0.2996 | −1.36 | 0.5 | −0.44 |
| 77 | Thpo | G05 | 0.55 | 0.3920 | −1.82 | 0.4 | −0.87 |
| 78 | Tnf | G06 | 0.25 | 0.0181 | −4.00 | 1.7 | −2.00 |
| 79 | Tnfrsf11b | G07 | 0.25 | 0.0007 | −4.07 | 3.2 | −2.02 |
| 80 | Tnfsf10 | G08 | 1.02 | 0.7133 | 1.02 | 0.1 | 0.03 |
| 81 | Tnfsf11 | G09 | 0.38 | 0.1108 | −2.61 | 1.0 | −1.39 |
| 82 | Tnfsf13b | G10 | 0.88 | 0.6833 | −1.13 | 0.2 | −0.18 |
| 83 | Vegfa | G11 | 0.40 | 0.0058 | −2.52 | 2.2 | −1.33 |
| 84 | Xcl1 | G12 | 1.47 | 0.2829 | 1.47 | 0.5 | 0.56 |

TABLE 2
Liver cancer mRNA expression associated with increased
(HR <1) or decreased (HR >1) overall survival of HCC
patients (n = 364) at 3 years by Kaplan-Meier analysis.

| mRNA | HR | p |
|---|---|---|
| CA9 | 2.49 | 6.0E−6 |
| CXCL1 | 1.49 | 4.5E−2 |
| CXCL2 | 0.51 | 8.5E−4 |
| CXCL9 | 0.63 | 2.0E−2 |
| CXCL10 | 0.61 | 1.4E−2 |
| EPO | 1.67 | 9.8E−3 |
| KITLG | 2.28 | 4.8E−5 |
| LDHA | 2.35 | 2.6E−5 |
| PGF | 1.50 | 4.1E−2 |
| SLC2A1 | 1.73 | 5.7E−3 |

TABLE 3
PCR primers for amplification of human (H) and mouse
(m) sequences.

| | SEQ ID | | | |
|---|---|---|---|---|
| Oligo Name | NO(s) | Sequence | Source | Application |
| H-ANGPTL4-F | 1 | GGACACGGCCTATAGCCTG | | RT-qPCR |
| H-ANGPTL4-R | 2 | CTCTTGGCGCAGTTCTTGTC | | RT-qPCR |
| H-VEGFA-F | 3 | AGGGCAGAATCATCACGAAGT | | RT-qPCR |
| H-VEGFA-R | 4 | AGGGTCTCGATTGGATGGCA | | RT-qPCR |
| H-CA9-F | 5 | TCTCGTTTCCAATGCACGTACAGC | | RT-qPCR |
| H-CA9-R | 6 | AGTGACAGCAGCAGTTGCACAGT | | RT-qPCR |
| H-EPO-F | 7 | GGAGGCCGAGAATATCACGAC | | RT-qPCR |
| H-EPO-R | 8 | CCCTGCCAGACTTCTACGG | | RT-qPCR |
| H-NDRG1-F | 9 | CTCCTGCAAGAGTTTGATGTCC | | RT-qPCR |
| H-NDRG1-R | 10 | CATGCCGATGTCATGGTAGG | | RT-qPCR |
| H-SDF-1-F | 11 | ATTCTCAACACTCCAAACTGTGC | | RT-qPCR |
| H-SDF-1-R | 12 | ACTTTAGCTTCGGGTCAATGC | | RT-qPCR |
| H-PGF-F | 13 | GAACGGCTCGTCAGAGGTG | | RT-qPCR |
| H-PGF-R | 14 | ACAGTGCAGATTCTCATCGCC | | RT-qPCR |
| H-CD73-F | 15 | GCCTGGGAGCTTACGATTTTG | | RT-qPCR |
| H-CD73-R | 16 | TAGTGCCCTGGTACTGGTCG | | RT-qPCR |
| H-CD47-F | 17 | AGAAGGTGAAACGATCATCGAGC | | RT-qPCR |
| H-CD47-R | 18 | CTCATCCATACCACCGGATCT | | RT-qPCR |
| H-PDL1-F | 19 | CCAGGATGGTTCTTAGACTCCC | | RT-qPCR |
| H-PDL1-R | 20 | TTTAGCACGAAGCTCTCCGAT | | RT-qPCR |
| H-RPL13A-F | 21 | CTCAAGGTCGTGCGTCTG | | RT-qPCR |
| H-RPL13A-R | 22 | TGGCTTTCTCTTTCCTCTTCTC | | RT-qPCR |
| m-Angptl4-F | 23 | CATCCTGGGACGAGATGAACT | | RT-qPCR |
| m-Angptl4-R | 24 | TGACAAGCGTTACCACAGGC | | RT-qPCR |
| m-Epo-F | 25 | ACTCTCCTTGCTACTGATTCCT | | RT-qPCR |
| m-Epo-R | 26 | ATCGTGACATTTTCTGCCTCC | | RT-qPCR |
| m-Pgf-F | 27 | TCTGCTGGGAACAACTCAACA | | RT-qPCR |
| m-Pgf-R | 28 | GTGAGACACCTCATCAGGGTAT | | RT-qPCR |
| m-Rpl13a-F | 29 | GGGCAGGTTCTGGTATTGGAT | | RT-qPCR |
| m-Rpl13a-R | 30 | GGCTCGGAAATGGTAGGGG | | RT-qPCR |
| m-18S-F | 31 | GTAACCCGTTGAACCCCATT | | RT-qPCR |
| m-18S-R | 32 | CCATCCAATCGGTAGTAGCG | | RT-qPCR |
| m-Il6-F | 33 | TAGTCCTTCCTACCCCAATTTCC | | RT-qPCR |
| m-Il6-R | 34 | TTGGTCCTTAGCCACTCCTTC | | RT-qPCR |
| m-Il11-F | 35 | TGTTCTCCTAACCCGATCCCT | | RT-qPCR |
| m-Il11-R | 36 | CAGGAAGCTGCAAAGATCCCA | | RT-qPCR |
| m-Il22-F | 37 | ATGAGTTTTTCCCTTATGGGGAC | | RT-qPCR |
| m-Il22-R | 38 | GCTGGAAGTTGGACACCTCAA | | RT-qPCR |
| m-Cd70-F | 39 | TGTAGCGGACTACTCAGTAAGC | | RT-qPCR |
| m-Cd70-R | 40 | TGGGGTCCTTCCGAGGAAC | | RT-qPCR |
| m-Slc2a1-F | 41 | CAGTTCGGCTATAACACTGGTG | | RT-qPCR |
| m-Slc2a1-R | 42 | GCCCCCGACAGAGAAGATG | | RT-qPCR |
| m-Ldha-F | 43 | TGTCTCCAGCAAAGACTACTGT | | RT-qPCR |
| m-Ldha-R | 44 | GACTGTACTTGACAATGTTGGGA | | RT-qPCR |
| m-B7-h4-F | 45 | CTTTGGCATTTCAGGCAAGCA | | RT-qPCR |
| m-B7-h4-R | 46 | TGATGTCAGGTTCAAAAGTGCAG | | RT-qPCR |
| CA9-HRE-F | 47 | GACAAACCTGTGAGACTTTGGCTC | | ChIP-qPCR |
| | | C | | |
| CA9-HRE-R | 48 | AGTGACAGCAGCAGTTGCACAGTG | | ChIP-qPCR |
| ANGPTL4-HRE-F | 49 | ATTTGCTGTCCTGGCATC | | ChIP-qPCR |
| ANGPTL4-HRE-R | 50 | CCAGCTCATTCTCTGGAATC | | ChIP-qPCR |
| PDK1-HRE-F | 51 | CGCGTTTGGATTCCGTG | | ChIP-qPCR |
| PDK1-HRE-R | 52 | CCAGTTATAATCTGCCTTCCCTATT | | ChIP-qPCR |
| | | ATC | | |
| RPL13A-F | 53 | GAGGCGAGGGTGATAGAG | | ChIP-qPCR |
| RPL13A-R | 54 | ACACACAAGGGTCCAATTC | | ChIP-qPCR |

TABLE 4
Antibodies for ChIP, immunoprecipitation (IP), immunoblot
(IB) assays and ELISAs for quantification of human (h), mouse
(m), and rabbit (r) proteins; fluorescently labeled antibodies
for flow cytometry (FC); and antibodies for in vivo studies (IV).

| Target protein | Catalog number | Vendor | Application |
|---|---|---|---|
| h-HIF-1α | NB100-479 | Novas Biologicals | ChIP |
| L-HIF-1β | NB100-110 | Novus Biologicals | IB, IP, |
| | | | ChIP |
| h-HIF-2α | NB100-122 | Novus Biologicals | IB, ChIP |
| h-p300 | NB500-161 | Novus Biologicals | IB, ChIP |
| m-β-Actin | Sc-47778 | Santa Cruz | IB |
| r-IgG | NBP2-36463 | Novus Biologicals | IB, IP |
| HRP-anti-r-IgG | NA934V | ThermoFisher | IB |
| HRP-anti-r-IgG | NA931V | ThermoFisher | IB |
| m-CcI2 | NBP1-92659 | Novus Biologicals | ELISA |
| m-Cxc11 | DY453 | Novus Biologicals | ELISA |
| m-Cxc12 | MM200 | Novus Biologicals | ELISA |
| m-Cxc15 | MX000 | Novus Biologicals | ELISA |
| m-Cxc19 | MCX900 | Novus Biologicals | ELISA |
| m-Cxc110 | DY466 | Novus Biologicals | ELISA |
| m-Ifng | MIF00 | Novus Biologicals | ELISA |
| m-I11b | MLB00C | Novus Biologicals | ELISA |
| m-I16 | M6000B | Novus Biologicals | ELISA |
| m-I19 | NBP3-06772 | Novus Biologicals | ELISA |
| m-I110 | DY417 | Novus Biologicals | ELISA |
| m-I111 | NBP3-06794 | Novus Biologicals | ELISA |
| m-I122 | M2200 | Novus Biologicals | ELISA |
| m-Vegfa | MMV00 | Novus Biologicals | ELISA |
| h-EPO | DEP00 | Novus Biologicals | ELISA |
| h-SCF | DCK00 | Novus Biologicals | ELISA |
| h-SDF-1α | DSA00 | Novus Biologicals | ELISA |
| h-VEGFA | DVE00 | Novus Biologicals | ELISA |
| m-Cd3 | FAB4841G | Novus Biologicals | FC |
| m-Cd4 | FAB554A | Novus Biologicals | FC |
| m-Cd8A | NBP1-49045-PE | Novus Biologicals | FC |
| m-Cd11b | NB110-89474-AF405 | Novus Biologicals | FC |
| m-Cd11c | NB110-40766-AF488 | Novus Biologicals | FC |
| m-Cd25 | NBP2-27425-AF488 | Novus Biologicals | FC |
| m-Cd44 | NBP1-47386-APC | Novus Biologicals | FC |
| m-Cd45 | NB100-77417-AF405 | Novus Biologicals | FC |
| m-Cd69 | NBP1-28011-AF488 | Novus Biologicals | FC |
| m-Cd80 | NBP1-43385-AF488 | Novus Biologicals | FC |
| m-Cd314 | FAB 1547V | Novus Biologicals | FC |
| m-F4/80 | NB600-404-APC | Novus Biological | FC |
| m-Foxp3 | NB100-39002-PE | Novus Biologicals | FC |
| m-Ifng | IC485V | Novus Biologicsis | FC |
| m-Ly6c | NBP1-28046-AF488 | Novus Biologicals | FC |
| m-Ly6g | FAB1037A | Novus Biologicals | FC |
| m-Nk1.1 | NB100-77528-APC | Novus Biologicals | FC |
| Rat IgG2a, | BE0089 | Bio X Cell | IV |
| x, anti-m- | | | |
| PD1 | | | |
| Rat anti-m-IgG2a, | Bio X Cell | Bio X Cell | IV |
| x Isotype | | | |
| control | | | |

TABLE 5
Antibodies for immunofluorescence (IF) and Western Blot (WB)

| | | | Catalog | | |
|---|---|---|---|---|---|
| Name | Company | Species | Number | dilution | Applications |
| Isolectin GS IB4 | Thermo Fisher | | I21413 | 1:200 | IF |
| HIF-1α | Gene Tex | rabbit | GTX127309 | 1:1000 | WB |
| HIF-2α | Gene Tex | mouse | GTX632015 | 1:1000 | WB |
| α/β tubulin | Cell signaling | rabbit | 2148s | 1:5000 | WB |
| RBPMS | Abcam | rabbit | ab152101 | 1:200 | IF |
| CRALBP | Abcam | mouse | ab15051 | 1:500 | IF |
| REC | MilliporeSigma | rabbit | AB5585 | 1:500 | IF |
| PAX-6 | DSHB | mouse | AB_528427 | 1:50 | IF |
| secondary | Invitrogen | | | 1:1000 | IF |
| antibodies | | | | | |

TABLE 6
Primer sequences for Real-Time (RT)-PCR

| | Gene | | Sequence (5′ to 3′) |
|---|---|---|---|
| Mouse | | | |
| | ANGPTL4 | Forward | TTGGTACCTGTAGCCATTCC (SEQ ID NO: 55) |
| | | Reverse | GAGGCTAAGAGGCTGCTGTA (SEQ ID NO: 56) |
| | VEGF | Forward | TCCTCCTATCTCCACC (SEQ ID NO: 57) |
| | | Reverse | GACCCAGCCAGCCATA (SEQ ID NO: 58) |
| | Cyclophilin A | Forward | AGCATACAGGTCCTGGCATC (SEQ ID NO: 59) |
| | | Reverse | TTCACCTTCCCAAAGACCAC (SEQ ID NO: 60) |
| | PAI-1 | Forward | TGATGGCTCAGAGCAACAAG (SEQ ID NO: 61) |
| | | Reverse | GCCAGGGTTGCACTAAACAT (SEQ ID NO: 62) |
| | ANGPT2 | Forward | TCCAAGAGCTCGGTTGCTAT (SEQ ID NO: 63) |
| | | Reverse | AGTTGGGGAAGGTCAGTGTG (SEQ ID NO: 64) |
| | VE-PTP | Forward | TCAAGGCAGGACAGTACCC (SEQ ID NO: 65) |
| | | Reverse | TGTATTTCTCCCATTCGCCTAGA (SEQ ID NO: 66) |
| | PDGFA | Forward | GAGATACCCCGGGAGTTGAT (SEQ ID NO: 67) |
| | | Reverse | TCTTGAAAACTGGAGGAATG (SEQ ID NO: 68) |
| | KDR | Forward | GCAGAAGATACTGTCACCACC (SEQ ID NO: 69) |
| | | Reverse | TTTGGCAAATACAACCCTTCAGA (SEQ ID NO: 70) |
| | EPO | Forward | ACTCTCCTTGCTACTGATTCCT (SEQ ID NO: 25) |
| | | Reverse | ATCGTGACATTTTCTGCCTCC (SEQ ID NO: 26) |
| Human | | | |
| | ANGPTL4 | Forward | GGACACGGCCTATAGCCTG (SEQ ID NO: 1) |
| | | Reverse | CTCTTGGCGCAGTTCTTGTC (SEQ ID NO: 2) |
| | VEGF | Forward | GGGCAGAATCATCACGAAGT (SEQ ID NO: 71) |
| | | Reverse | TGGTGATGTTGGACTCCTCA (SEQ ID NO: 72) |
| | Cyclophilin A | Forward | TTCATCTGCACTGCCAAGAC (SEQ ID NO: 73) |
| | | Reverse | TCGAGTTGTCCACAGTCAGC (SEQ ID NO: 74) |
| | PAI-1 | Forward | ACAACAGGAGAAACCCA (SEQ ID NO: 75) |
| | | Reverse | CACGTCATGGGTGGTTTCTTG (SEQ ID NO: 76) |
| | ANGPT2 | Forward | GCAAGTGCTGGAGAACATCA (SEQ ID NO: 77) |
| | | Reverse | GTTAACTTCCGCGTTTGCTC (SEQ ID NO: 78) |
| | VE-PTP | Forward | ACAACACCACATACGGATGTAAC (SEQ ID NO: 79) |
| | | Reverse | CCTAGCAGGAGGTAAAGGATCT (SEQ ID NO: 80) |

TABLE 7
HIF transcriptional activity

| Compound ID | IC₅₀/μM |
|---|---|
| 11-88 | 2.9 |
| 32-134C | 2.3 |
| 32-134D | 2.5 |
| 12-143 | 2.6 |
| 33-063 | 2.2 |
| 33-093B | 2.7 |
| 11-147B | 2.92 |
| 12-58 | 3.09 |
| 33-057A | 2.86 |
| 12-70C | 2.31 |
| 12-93A | 2.40 |
| 12-93F | 2.48 |
| 33-087 | 2.51 |
| 33-103B | 2.87 |
| 33-103C | 2.80 |
| 33-123 | 3.24 |
| 12-147A | 2.52 |
| 12-147B | 3.08 |
| 34-090 | 1.83 |
| 34-110 | 1.27 |
| 34-122 | 2.02 |
| 34-146 | 2.04 |
| 35-020 | 2.37 |
| 35-092 | 2.07 |
| 35-140 | 1.94 |
| 13-01 | 2.07 |
| 13-03 | 2.69 |

1. A method of treating a condition or disease in which inhibiting HIF-1 and/or HIF-2 is beneficial, the method comprising administering to a subject in need of such treatment a compound of Formula (I): 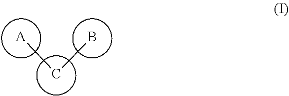
or a pharmaceutically acceptable salt thereof, wherein:
Ring A is heteroaryl including from 8-10 ring atoms, wherein from 1-4 ring atoms are heteroatoms each independently selected from the group consisting of: N, N(R^{a}), O, and S(O)₀₋₂, which is optionally substituted with from 1-4 X^{a};
Ring B is heteroaryl including from 8-10 ring atoms, wherein from 1-4 ring atoms are heteroatoms each independently selected from the group consisting of: N, N(R^{a}), O, and S(O)₀₋₂, which is optionally substituted with from 1-4 X^{b};
Ring C is heteroaryl including from 5-6 ring atoms, wherein from 1-4 ring atoms are heteroatoms each independently selected from the group consisting of: N, N(R^{a}), O, and S(O)₀₋₂, which is optionally substituted with from 1-2 R^{b};
each X^{a} and X^{b} is independently selected from the group consisting of: halo, C₁₋₆ alkyl, C₁₋₆ haloalkyl, C₂₋₆ alkenyl, C₂₋₆ alkynyl, NO₂, cyano, C₁₋₆ alkoxy, C₁₋₆ haloalkoxy, SO₂(C₁₋₆ alkyl), heterocyclyl, heteroaryl, C₃₋₆ cycloalkyl, NR¹R², C(O)R³, and C(O)NR¹R²;
each occurrence of R^{a} is independently selected from the group consisting of: H, C₁₋₄ alkyl, —C(═O)R³, —C(═O)OR³, —C(═O)NR¹R², and —SO₂R⁴;
each occurrence of R^{b} is independently selected from the group consisting of: H, C₁₋₄ alkyl, C₁₋₄ haloalkyl, halo, and cyano;
each occurrence of R¹ and R² is independently selected from the group consisting of: H, C₁₋₄ alkyl, optionally substituted C₃₋₆ cycloalkyl, optionally substituted phenyl, optionally substituted benzyl, C(═O)R⁵, C(═O)OR⁵, and C(═O)NR⁵R⁶; or
a pair of R¹ and R² on the same nitrogen atom, taken together with said nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of: N, N(R^{a}), O, and S;
each occurrence of R³ is independently selected from the group consisting of: H, C₁₋₄ alkyl, C₁₋₄ haloalkyl, optionally substituted C₃₋₆ cycloalkyl, and optionally substituted phenyl;
each occurrence of R⁴ is independently selected from the group consisting of: C₁₋₄ alkyl, C₁₋₄ haloalkyl, optionally substituted C₃₋₆ cycloalkyl, optionally substituted phenyl; and optionally substituted benzyl; and
each occurrence of R⁵ and R⁶ is independently selected from the group consisting of: H, C₁₋₄ alkyl.

2. A method of treating a condition or disease that is associated with hypoxia, or with increased expression of hypoxia-inducible factor HIF-1α and/or HIF-2α, or with both, the method comprising administering to a subject in need of such treatment a compound of Formula (I) that is capable of inhibiting transcriptional activation mediated by HIFs.

3. The method of claim 1, wherein the method comprises administering a second therapeutic agent.

4. The method of claim 3, wherein the second therapeutic agent is a chemotherapeutic agent.

5-8. (canceled)

9. The method of claim 1, wherein the condition of disease is cancer.

10. (canceled)

11. A method of treating cancer, method comprising administering to a subject in need of such treatment an inhibitor of HIF-1 and/or HIF-2 and a chemotherapeutic immunomodulatory agent, optionally wherein the chemotherapeutic immunomodulatory agent is an immune checkpoint inhibitor.

12. (canceled)

13. The method of claim 1, wherein Ring A is 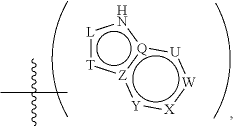
wherein:
L and T is independently C*, N*, CR⁵, N, O, or S;
Z and Q is independently N and C; and
U, W, X, and Y is independently C*, N*, CR⁶ and N; wherein
C* and N* denote attachment points to Ring C.

14-15. (canceled)

16. The method of claim 1, wherein Ring A is selected from the group consisting of

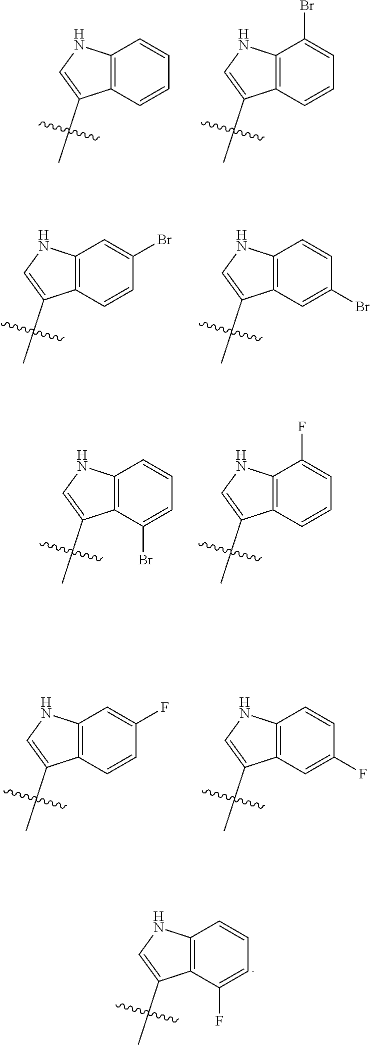

17. (canceled)

18. The method of claim 1, wherein Ring B is 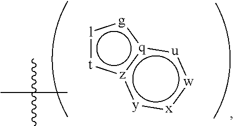
wherein:
l, t and g is C*, N*, CR⁵, NR⁵, N, O, C(═O), or S;
z and q is independently N and C; and
u, w, x, and y is independently C*, N*, CR⁶ and N; wherein
C* and N* denote attachment points to Ring C.

19-20. (canceled)

21. The method of claim 1, wherein Ring B is selected from the group consisting of

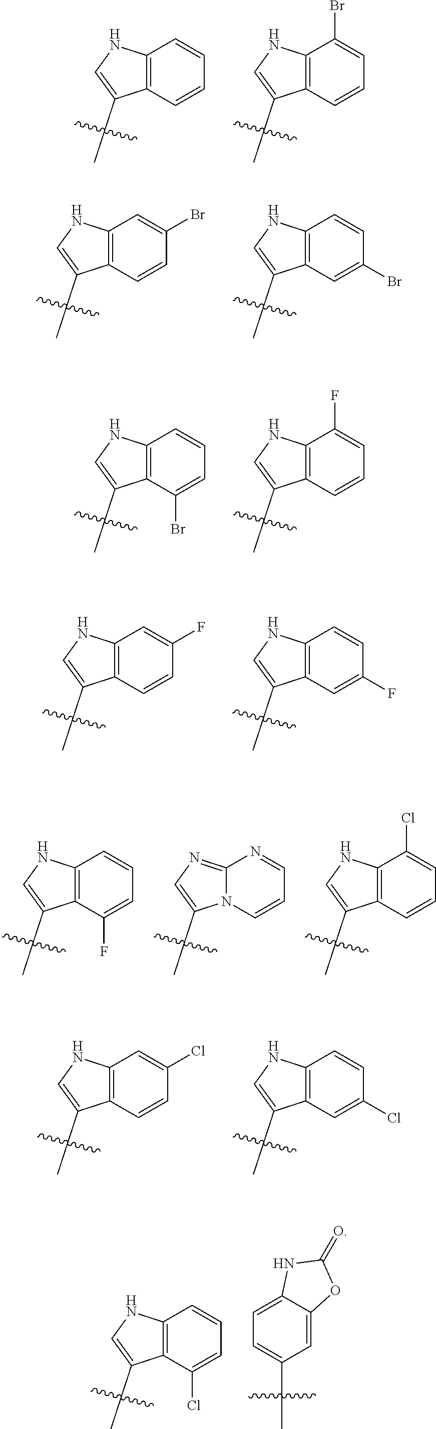

22. (canceled)

23. The method of claim 1, wherein Ring C is 5-membered heteroaryl selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

24. (canceled)

25. The method of claim 1, wherein Ring C is 5-membered heteroaryl selected from the group consisting of 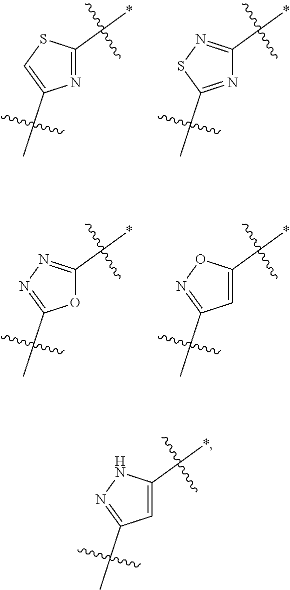
wherein the asterisk indicates the point of attachment to ring A.

26. (canceled)

27. The method of claim 1, wherein Ring C is 6-membered heteroaryl selected from the group consisting of pyridinyl, pyrimidinyl, pyrazinyl, pyridazinyl, and triazinyl.

28. The method of claim 1, wherein Ring C is 6-membered heteroaryl selected from the group consisting of 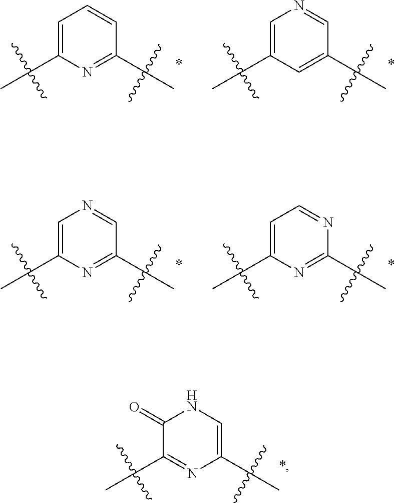 [SMILES: *c1cccc(*)n1 ; *c1cncc(*)c1 ; *c1cncc(*)n1 ; *c1ccnc(*)n1 ; *c1c[nH]c(=O)c(*)n1]
wherein the asterisk indicates the point of attachment to ring A.

29. The method of claim 1, wherein the compound is Formula (II) 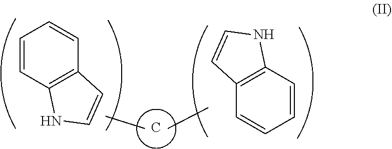
or a pharmaceutically acceptable salt thereof.

30. The method of claim 1, wherein the compound is Formula (II′) 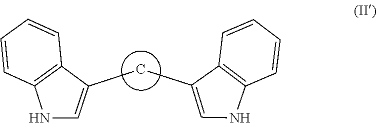 [SMILES: c1ccc2c(Cc3c[nH]c4ccccc34)c[nH]c2c1]
or a pharmaceutically acceptable salt thereof.

31-33. (canceled)

34. The method of claim 1, wherein the compound is 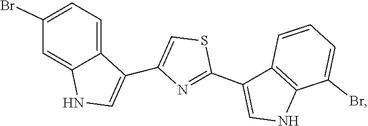 [SMILES: Brc1ccc2c(-c3csc(-c4c[nH]c5c(Br)cccc45)n3)c[nH]c2c1]
or a pharmaceutically acceptable salt thereof.

35. The method of claim 1, wherein the compound is 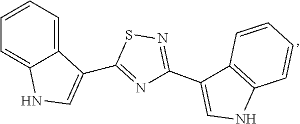 [SMILES: c1ccc2c(-c3nsc(-c4c[nH]c5ccccc45)n3)c[nH]c2c1]
or a pharmaceutically acceptable salt thereof.

36. The method of claim 1 selected from Table C1.

37. A method of treating blinding eye disease, method comprising administering to a subject in need of such treatment an inhibitor of HIF-1 and/or HIF-2.

38. The method of claim 37, wherein the blinding eye disease is selected from the group consisting of dry AMD, wet AMD, ischemic retinopathy, diabetic retinopathy, retinal vein occlusion, sickle cell retinopathy, and retinopathy of prematurity.

39-44. (canceled)

45. A compound having the formula 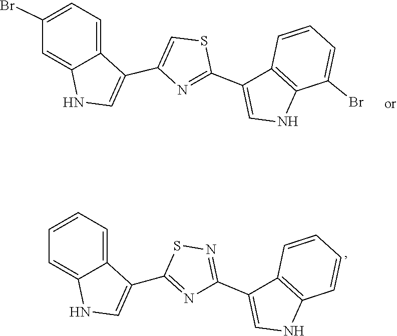 [SMILES: Brc1ccc2c(-c3csc(-c4c[nH]c5c(Br)cccc45)n3)c[nH]c2c1 ; "or" ; c1ccc2c(-c3nsc(-c4c[nH]c5ccccc45)n3)c[nH]c2c1 ; ","]
or a pharmaceutically acceptable salt thereof.

46. (canceled)
